SEQUENCE LISTING (73 sequences)

SEQ ID NO: 1 - DNA, 31 bp, synthetic construct
  source 1..31: mol_type = other DNA
gatcggaagagcacacgtctgaactccagtc

SEQ ID NO: 2 - DNA, 29 bp, synthetic construct
  source 1..29: mol_type = other DNA
agtggttggtgagggtcatctcgctggag

SEQ ID NO: 3 - DNA, 29 bp, synthetic construct
  source 1..29: mol_type = other DNA
acaaaggcagccacgcactccttccctgt

SEQ ID NO: 4 - DNA, 33 bp, synthetic construct
  source 1..33: mol_type = other DNA
acactctttccctacacgacgctcttccgatct

SEQ ID NO: 5 - DNA, 19 bp, synthetic construct
  source 1..19: mol_type = other DNA
aatgatacggcgaccaccg

SEQ ID NO: 6 - DNA, 21 bp, synthetic construct
  source 1..21: mol_type = other DNA
caagcagaagacggcatacga

SEQ ID NO: 7 - DNA, 40 bp, synthetic construct
  source 1..40: mol_type = other DNA
catcctttcagagaaaataatgctcctagtacctgtagag

SEQ ID NO: 8 - DNA, 40 bp, synthetic construct
  source 1..40: mol_type = other DNA
tatagatggtgaaacctgtttgttggacatactggataca

SEQ ID NO: 9 - DNA, 35 bp, synthetic construct
  source 1..35: mol_type = other DNA
atccgacaagtgagagacaggatcaggtcagcggg

SEQ ID NO: 10 - DNA, 39 bp, synthetic construct
  source 1..39: mol_type = other DNA
gctggtgtgaaatgactgagtacaaactggtggtggttg

SEQ ID NO: 11 - DNA, 35 bp, synthetic construct
  source 1..35: mol_type = other DNA
gctgccagccaggaagcgggagggaaggctcactc

SEQ ID NO: 12 - DNA, 39 bp, synthetic construct
  source 1..39: mol_type = other DNA
gattcagacagtggactggagcagatgtccattgggcat

SEQ ID NO: 13 - DNA, 54 bp, synthetic construct
  source 1..54: mol_type = other DNA
tttactaaatattgttttatttcctagtatagcataattgagagaaaaactgat

SEQ ID NO: 14 - DNA, 39 bp, synthetic construct
  source 1..39: mol_type = other DNA
acaaaacaggctcaggacttagcaagaagttatggaatt

SEQ ID NO: 15 - DNA, 43 bp, synthetic construct
  source 1..43: mol_type = other DNA
cagatctgtatttatttcagtgttacttacctgtcttgtcttt

SEQ ID NO: 16 - DNA, 39 bp, synthetic construct
  source 1..39: mol_type = other DNA
aagagttaaggactctgaagatgtacctatggtcctagt

SEQ ID NO: 17 - DNA, 45 bp, synthetic construct
  source 1..45: mol_type = other DNA
attactccttaatgtcagcttattatattcaatttaaacccacct

SEQ ID NO: 18 - DNA, 40 bp, synthetic construct
  source 1..40: mol_type = other DNA
agtagtaattgatggagaaacctgtctcttggatattctc

SEQ ID NO: 19 - DNA, 41 bp, synthetic construct
  source 1..41: mol_type = other DNA
tggtcagagaaacctttatctgtatcaaagaatggtcctgc

SEQ ID NO: 20 - DNA, 44 bp, synthetic construct
  source 1..44: mol_type = other DNA
tttttattataaggcctgctgaaaatgactgaatataaacttgt

SEQ ID NO: 21 - DNA, 38 bp, synthetic construct
  source 1..38: mol_type = other DNA
ctttccaacctaggaaggcaggggagtagggccaggaa

SEQ ID NO: 22 - DNA, 36 bp, synthetic construct
  source 1..36: mol_type = other DNA
tgttgctgcagatccgtgggcgtgagcgcttcgaga

SEQ ID NO: 23 - DNA, 37 bp, synthetic construct
  source 1..37: mol_type = other DNA
tctgaggcataactgcacccttggtctcctccaccgc

SEQ ID NO: 24 - DNA, 35 bp, synthetic construct
  source 1..35: mol_type = other DNA
acagctttgaggtgcgtgtttgtgcctgtctggga

SEQ ID NO: 25 - DNA, 37 bp, synthetic construct
  source 1..37: mol_type = other DNA
cctccaccgcttcttgtcctgcttgcttacctcgctt

SEQ ID NO: 26 - DNA, 37 bp, synthetic construct
  source 1..37: mol_type = other DNA
agtagtggtaatctactgggacggaacagctttgagg

SEQ ID NO: 27 - DNA, 37 bp, synthetic construct
  source 1..37: mol_type = other DNA
ctcctccaccgcttcttgtcctgcttgcttacctcgc

SEQ ID NO: 28 - DNA, 38 bp, synthetic construct
  source 1..38: mol_type = other DNA
ctgagtagtggtaatctactgggacggaacagctttga

SEQ ID NO: 29 - DNA, 38 bp, synthetic construct
  source 1..38: mol_type = other DNA
tatggaagaaatcggtaagaggtgggcccaggggtcag

SEQ ID NO: 30 - DNA, 38 bp, synthetic construct
  source 1..38: mol_type = other DNA
caccatccactacaactacatgtgtaacagttcctgca

SEQ ID NO: 31 - DNA, 36 bp, synthetic construct
  source 1..36: mol_type = other DNA
ggccagacctaagagcaatcagtgaggaatcagagg

SEQ ID NO: 32 - DNA, 36 bp, synthetic construct
  source 1..36: mol_type = other DNA
gccatggccatctacaagcagtcacagcacatgacg

SEQ ID NO: 33 - DNA, 38 bp, synthetic construct
  source 1..38: mol_type = other DNA
ccccaaaccccaatgaagagagaccagagcccagacct

SEQ ID NO: 34 - DNA, 40 bp, synthetic construct
  source 1..40: mol_type = other DNA
ctggatccctgatggggagaatgtgaaaattccagtggcc

SEQ ID NO: 35 - DNA, 35 bp, synthetic construct
  source 1..35: mol_type = other DNA
ctgggagcccaggcgtccgcggttttcccggacat

SEQ ID NO: 36 - DNA, 40 bp, synthetic construct
  source 1..40: mol_type = other DNA
ctctcagcgtacccttgtccccaggaagcatacgtgatgg

SEQ ID NO: 37 - DNA, 52 bp, synthetic construct
  source 1..52: mol_type = other DNA
caatttcataccttgcttaatgggtgtagataccaaaagataagaataaaac

SEQ ID NO: 38 - DNA, 34 bp, synthetic construct
  source 1..34: mol_type = other DNA
cccccggcttgtgagtggatgggtaaaacctatc

SEQ ID NO: 39 - DNA, 37 bp, synthetic construct
  source 1..37: mol_type = other DNA
atcggtctttgcctgctgagagttattaacagtgcag

SEQ ID NO: 40 - DNA, 41 bp, synthetic construct
  source 1..41: mol_type = other DNA
ccttagataaaactgagcaagaggctttggagtatttcatg

SEQ ID NO: 41 - DNA, 45 bp, synthetic construct
  source 1..45: mol_type = other DNA
ggaaacaatttttatcgaaagttgaaactaaaaatcctttgcagg

SEQ ID NO: 42 - DNA, 41 bp, synthetic construct
  source 1..41: mol_type = other DNA
ctccttactcatggtcggatcacaaagatttgtgattttgg

SEQ ID NO: 43 - DNA, 41 bp, synthetic construct
  source 1..41: mol_type = other DNA
gttttgaaaagagaacgcattaactcatcctgaattgtagc

SEQ ID NO: 44 - DNA, 40 bp, synthetic construct
  source 1..40: mol_type = other DNA
caggcagctgagtatcgagaaattgacaaacgtatgaaca

SEQ ID NO: 45 - DNA, 40 bp, synthetic construct
  source 1..40: mol_type = other DNA
ttatcaaacacggtagggaaagttctcactctgcattata

SEQ ID NO: 46 - DNA, 47 bp, synthetic construct
  source 1..47: mol_type = other DNA
tggttccttaaccacatttcttttttttttttccaggctattcaaga

SEQ ID NO: 47 - DNA, 42 bp, synthetic construct
  source 1..42: mol_type = other DNA
aggaactgaaaaagtaatttaaatgggaatagcccttcaata

SEQ ID NO: 48 - DNA, 38 bp, synthetic construct
  source 1..38: mol_type = other DNA
ctggaaaaaactgtttgggacctccggtcagaaaacca

SEQ ID NO: 49 - DNA, 41 bp, synthetic construct
  source 1..41: mol_type = other DNA
agagtaaagtagatgatggaaatatacagcttgcaaggact

SEQ ID NO: 50 - DNA, 41 bp, synthetic construct
  source 1..41: mol_type = other DNA
gaggatcttgaaggaaactgaattcaaaaagatcaaagtgc

SEQ ID NO: 51 - DNA, 39 bp, synthetic construct
  source 1..39: mol_type = other DNA
cagggtctagagcagagcagctgccagacatgagaaaag

SEQ ID NO: 52 - DNA, 40 bp, synthetic construct
  source 1..40: mol_type = other DNA
atcccagaaggtgagaaagttaaaattcccgtcgctatca

SEQ ID NO: 53 - DNA, 38 bp, synthetic construct
  source 1..38: mol_type = other DNA
ctgcacacaccagttgagcaggtactgggagccaatat

SEQ ID NO: 54 - DNA, 36 bp, synthetic construct
  source 1..36: mol_type = other DNA
gtgcctctccctccctccaggaagcctacgtgatgg

SEQ ID NO: 55 - DNA, 41 bp, synthetic construct
  source 1..41: mol_type = other DNA
gcatcctccccctgcatgtgttaaacaatacagctagtggg

SEQ ID NO: 56 - DNA, 40 bp, synthetic construct
  source 1..40: mol_type = other DNA
tactggtgaaaacaccgcagcatgtcaagatcacagattt

SEQ ID NO: 57 - DNA, 38 bp, synthetic construct
  source 1..38: mol_type = other DNA
ctttctagtaactcagcagcatctcagggccaaaaatt

SEQ ID NO: 58 - DNA, 38 bp, synthetic construct
  source 1..38: mol_type = other DNA
aagacctcacagtaaaaataggtgattttggtctagct

SEQ ID NO: 59 - DNA, 53 bp, synthetic construct
  source 1..53: mol_type = other DNA
tagtcctacagtgttttcagtttcaaaaatacttaactcctgttaaattatag

SEQ ID NO: 60 - DNA, 43 bp, synthetic construct
  source 1..43: mol_type = other DNA
gcaagctttctcacaagcatttggttttaaattatggagtatg

SEQ ID NO: 61 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 62 - protein, 248 aa, Homo sapiens
MFRFMRDVEPEDPMFLMDPFAIHRQHMSRMLSGGFGYSPFLSITDGNMPGTRPASRRMQQAGAVSPFGMLGMSGGFMDMFGMMNDMIGNMEHMTAGGNCQTFSSSTVISYSNTGDGAPKVYQETSEMRSAPGGIRETRRTVRDSDSGLEQMSIGHHIRDRAHILQRSRNHRTGDQEERQDYINLDESEAAAFDDEWRRETSRFRQQRPLEFRRLESSGAGGRRAEGPPRLAIQGPEDSPSRQSRRYDW

SEQ ID NO: 63 - protein, 189 aa, Homo sapiens [sequence omitted]

SEQ ID NO: 64 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 65 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 66 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 67 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 68 - protein, 976 aa, Homo sapiens [sequence omitted]

SEQ ID NO: 69 - protein, 361 aa, Homo sapiens
MHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 70 - protein, 259 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKAH

SEQ ID NO: 71 - protein, 1091 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 72 - protein, 766 aa, Homo sapiens [sequence omitted]

SEQ ID NO: 73 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

(19) United States
(12) Patent Application Publication
(10) Pub. No.: US 2026/0366075 A1
(43) Pub. Date: Nov. 19, 2026

(54) TARGETED AMPLIFICATION OF POLYNUCLEOTIDES

(71) Applicant: Singular Genomics Systems, Inc., San Diego, CA (US)

(72) Inventors: Eli N. GLEZER, Del Mar, CA (US); Daan WITTERS, San Diego, CA (US); Tung Thanh LE, San Diego, CA (US)

(21) Appl. No.: 19/082,820

(22) Filed: Mar. 18, 2025

Related U.S. Application Data
(60) Provisional application No. 63/568,374, filed on Mar. 21, 2024.

(51) Int. Cl. C12Q 1/6874; C12Q 1/6806; C12Q 1/6853

(52) U.S. Cl. CPC C12Q 1/6874; C12Q 1/6806; C12Q 1/6853

(57) ABSTRACT

Disclosed herein, inter alia, are compositions and methods for targeted amplification of polynucleotides and sequencing of polynucleotides.

### CROSS-REFERENCES TO RELATED APPLICATIONS

This application claims the benefit of U.S. Provisional Application No. 63/568,374, filed Mar. 21, 2024, which is incorporated herein by reference in its entirety and for all purposes.

### SEQUENCE LISTING

The instant application contains a Sequence Listing which has been submitted electronically in XML format and is hereby incorporated by reference in its entirety. Said XML copy, created on Mar. 4, 2025, is named 00617001US.xml, and is 74,031 bytes in size.

### BACKGROUND

In next-generation sequencing (NGS) applications, the ligation of exogenous adapters to pools of different target polynucleotides prior to sequencing is typically required. For example, a genomic polynucleotide sample is fragmented, end-repaired, and A-tailed, with purification steps occurring between most or all steps, all prior to ligation of an adapter at each end. The exogenous adapters are useful for immobilizing the target polynucleotides on a solid support and also serving as primer binding sites to initiate amplification and/or sequencing reactions. However, standard library preparation is both reagent- and time-consuming, and often is inefficient. For example, when ligating blunt end adapters to each end of the target polynucleotide the ligation efficiency of amplifiable and sequence-able products is less than about 50%. Disclosed herein, inter alia, are solutions to these and other problems in the art.

### BRIEF SUMMARY

In an aspect is a method of immobilizing a polynucleotide including: adding to a reaction vessel a first primer, a second primer, and a target polynucleotide, wherein the reaction vessel includes a solid support and a third primer attached to the solid support, wherein the first primer includes a first target binding sequence and a first platform primer binding sequence, the second primer includes a second target binding sequence and a second platform primer sequence, and the third primer includes a sequence complementary to the second platform primer sequence; and within the reaction vessel: (a) binding the first target binding sequence to the target polynucleotide and extending the first primer to generate a first complement of the target polynucleotide template includes the first platform primer binding sequence and a complementary sequence of the target polynucleotide; (b) binding the second target binding sequence to the first complement of the target polynucleotide and extending the second primer to generate a second template includes a first platform primer sequence, a sequence of the target polynucleotide, and the second platform primer sequence; and (c) binding the second platform primer sequence to the third primer and extending the third primer to form an immobilized polynucleotide.

In an aspect is a method of amplifying a polynucleotide, the method including: contacting a solid support of with a plurality of polynucleotides, wherein each of the plurality of polynucleotides include a first platform primer binding sequence, a target sequence, and a second platform primer sequence and do not include a common primer binding sequence, wherein the solid support includes a first plurality of immobilized oligonucleotides each including a first platform primer sequence and a second plurality of immobilized oligonucleotides each comprising a second platform primer sequence; hybridizing the first platform primer binding sequence of a polynucleotide of the plurality of polynucleotides to a first immobilized oligonucleotide from the first plurality, and extending with a polymerase the first immobilized oligonucleotide to generate a first extension product; hybridizing the first extension product to a second immobilized oligonucleotide from the second plurality, and extending with a polymerase the second immobilized oligonucleotide to generate a second extension product; and hybridizing the second extension product to a third immobilized oligonucleotide from the first plurality, and extending with a polymerase the third immobilized oligonucleotide to generate a third extension product. In embodiments, the method includes sequencing the first extension product, the second extension product, and/or the third extension product.

### BRIEF DESCRIPTION OF THE DRAWINGS

FIG. 1. An illustration of the different polynucleotides used as input for traditional sequencing platforms (top) compared to the input for the onboard target capture methods described herein (bottom). Typically, a target insert requires adapter ligation to render it usable on commercial sequencing platforms. An adapter (e.g., an exogenous adapter) may include a platform primer sequence (PP1 and PP2) such as the universal S1 and S2 sequences, a sequencing primer binding sequence (SP1 and SP2), and optionally one or two barcode/indexes (BC1 and BC2). In contrast, employing the methods described herein requires no adapter ligation to the input polynucleotide.

FIG. 2 shows examples of the primer oligonucleotide sequences, referred to as P1 and P2, respectively. The P1 primer contains a first platform primer sequence (pp1), which is a sequence complementary to a first immobilized primer (e.g., an oligonucleotide attached to a solid support), an optional index sequence, a first sequencing primer (SP1) sequence, and a first target hybridization sequence (T1′). The P2 primer contains a second platform primer sequence (pp2), which is a sequence complementary to a second immobilized primer, an optional index sequence, and a second sequencing primer (SP2) sequence, and a second target hybridization sequence (T2). Though not included in the illustration, it is understood that in embodiments, the complementary sequences (e.g., SP1′) may be substituted for the illustrated sequence (e.g., SP1). The dashed lines are indicative of regions within the primer and are included to aid the eye in the different arrangement of the sequences and are not indicative of the overall size/length (i.e., the index sequence may not be the same length as the sequencing primer despite the illustration showing the index sequence and sequencing primer as being the same size. In embodiments, the 5′ end of the primer includes the platform primer sequence.

FIGS. 3A-3D depict an example workflow to append functional sequences to a target nucleic acid molecule. Within the flow cell, a first primer oligonucleotide (e.g., a P1 oligonucleotide illustrated in FIG. 2) binds to a target molecule. The primer oligonucleotide is extended with a polymerase to provide a complement of the original target molecule with the pp1 sequence attached on the 5′ end. A second primer oligonucleotide (e.g., a P2 oligonucleotide illustrated in FIG. 2) binds to the complement target molecule and is extended with a polymerase to provide a nucleic acid molecule including both pp1′ and pp2 at the ends, referred to herein as a library molecule. The library molecules may be subjected to additional rounds of solution-phase amplification. Subsequent binding and extension (FIG. 3B) immobilizes the library molecule(s) to a flow cell containing immobilized platform primers, generating a complement of the library molecule. FIG. 3C depicts a first solid-phase amplification cycle, whereby the immobilized complement molecule anneals to immobilized oligonucleotide and is extended to generate immobilized sample polynucleotides. This solid phase amplification process is repeated (FIG. 3D) to yield features containing a plurality of immobilized sample polynucleotides.

FIG. 4 provides an example of the onboard amplification methods described herein. Minimally processed nucleic acid fragments are introduced into a flow cell. Solution phase primers are introduced and following cycles of amplification (e.g., 2-20 cycles), the nucleic acid fragments include functional sequences capable of binding to the immobilized nucleotides in the flow cell. Subsequent cycles of solid-phase amplification provide monoclonal clusters (i.e., features) of amplification products, which may be detected (e.g., sequenced).

### DETAILED DESCRIPTION

The aspects and embodiments described herein relate to compositions and methods for efficient amplification and sequencing of nucleic acid templates while minimizing library preparation reagents, techniques, and methods.

### I. DEFINITIONS

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support (e.g. a receiving substrate). An association may refer to a relationship, or connection, between two entities. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. The term “associated” or “associated with” as used herein to describe a disease (e.g. a protein associated disease, a cancer associated with aberrant NRAS activity) means that the disease (e.g., cancer) is caused by, or a symptom of the disease is caused by, what is described as disease associated or what is described as associated with the disease. For example, a cancer associated with aberrant NRAS activity may be a cancer that results (entirely or partially) from aberrant NRAS activity or a cancer wherein a particular symptom of the disease is caused (entirely or partially) by aberrant NRAS activity. As used herein, what is described as being associated with a disease, if a causative agent, could be a target for treatment of the disease. For example, a cancer associated with aberrant NRAS activity or an NRAS associated cancer, may be treated with a NRAS modulator or NRAS inhibitor, in the instance where increased NRAS activity causes the cancer. For example, a cancer associated with NRAS may be a cancer in a subject with a mutated NRAS gene is at higher risk of developing cancer as compared to a subject without a mutated NRAS gene.

As used herein, the term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a particle described herein to interact with an array.

As may be used herein, the terms “nucleic acid,” “nucleic acid molecule,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

As used herein, the term “mutated” refers to a gene or allele that has sequence modifications compared to the natural (i.e., wild type) gene or allele. The term “mutation” refers to a change in the nucleotide sequence of a normally conserved nucleic acid sequence that results in the formation of a differentiated mutant of the normal (unaltered) or natural sequence. Mutations can be divided, in general, into two general classes, namely, base pair substitutions (e.g., single nucleotide substitutions) and mutations with reading frame shift. The latter involves the insertion or deletion of one to several pairs of nucleotides.

As used herein, the term “allele” refers to two sequences that are different in only one or a few bases.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis. In embodiments, a primer includes a target binding sequence and a platform primer binding sequence (or complement thereof).

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55 °C to about 65 °C.

As used herein, a platform primer is a primer oligonucleotide immobilized or otherwise bound to a solid support (i.e. an immobilized oligonucleotide). Examples of platform primers include P7 and P5 primers, or S1 and S2 sequences, or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer.

As used herein, the term “capture domain” refers to an oligonucleotide sequence (e.g., an oligonucleotide sequence included in a primer, for example a surface-immobilized capture primer). The capture domain may be any suitable domain capable of hybridizing to RNA or a transcript thereof, such as mRNA. In some embodiments, the capture domain includes a poly-T oligonucleotide. A poly-T oligonucleotide includes a series of consecutive deoxythymidine residues linked by phosphodiester bonds. A poly-T oligonucleotide is capable of hybridizing to the poly-A tail of mRNA. In some embodiments, the capture domain may further include additional sequences to facilitate the capture of a particular RNA (e.g., mRNA) corresponding to select genes or groups of genes. Such a capture primer may be selected or designed based on sequence of the RNA it is desired to capture. Accordingly, the capture primer may be a sequence-specific capture primer as described herein. In some embodiments, the capture domain may target DNA, instead of RNA. In some embodiments, the capture domain may target non-specific or specific DNA sequences. For example, the capture domain may include a nucleic acid sequence to facilitate the capture of a target DNA sequence. The type of target may depend on the specific capture domain used and/or the presence of additional capture moieties on the substrate. For example, capture domains including a poly-dT tail are suited for spatial detection of RNA with poly-A tail. RNA that does not have poly-A tail may be labeled with poly-A before being captured by the substrate. Capture domains including a nucleic acid sequence against a target DNA sequence are useful for spatial detection of DNA. Substrates include a capture primer and an additional capture moiety (e.g. an antibody targeting protein or DNA/RNA probes targeting specific nucleic acid sequence) are useful for multiplex detection of nucleic acid and non-nucleic acid targets.

As used herein, the term “spatial barcode” refers to a known nucleic acid sequence that allows the location of a biological molecule with which the barcode is associated to be resolved. A barcode can be a spatial barcode. The barcode or spatial barcode may be associated with an oligonucleotide as described herein (e.g., a capture oligonucleotide or capture probe). The barcodes can be designed for precision sequence performance, e.g., GC content between 40% and 60%, no homo-polymer runs longer than two, no self-complementary stretches longer than 3, and be comprised of sequences not present in a human genome reference. A barcode sequence can be at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence can be at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. A barcode sequence can be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 bases. An oligonucleotide (e.g., primer or adapter) can include about, more than, less than, or at least 1, 2, 3, 4, 5, 6, 7, 8, 9, or 10 different barcodes. Barcodes can be of sufficient length and include sequences that can be sufficiently different to allow the identification of the spatial position of each biological molecule based on barcode(s) with which each biological molecule is associated. In some cases, each barcode is, for example, four deletions or insertions or substitutions away from any other barcode in an array. The oligonucleotides in each array spot on the barcoded oligonucleotide array can include the same barcode sequence and oligonucleotides in different array spots can include different barcode sequences. The barcode sequence used in one array spot can be different from the barcode sequence in any other array spot. Alternatively, the barcode sequence used in one array spot can be the same as the barcode sequence used in another array spot, as long as the two array spots are not adjacent. Barcode sequences corresponding to particular array spots can be known from the controlled synthesis of the array. Alternatively, barcode sequences corresponding to particular array spots can be known by retrieving and sequencing material from particular array spots. In embodiments, the spatial barcode sequence is indicative of the location of the immobilized capture probe on the solid support to within about 2 μm, about 1 μm, about 0.5 μm, about 0.2 μm, or about 0.1 μm.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the terms “analogue” and “analog”, in reference to a chemical compound, refers to compound having a structure similar to that of another one, but differing from it in respect of one or more different atoms, functional groups, or substructures that are replaced with one or more other atoms, functional groups, or substructures. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used herein, “endogenous” is used in accordance with its ordinary meaning in the art and refers to an internal origin. For example, an endogenous gene sequence (also referred to herein as an endogenous region) is a polynucleotide sequence found within the original polynucleotide sequence. In embodiments, an endogenous gene sequence is a polynucleotide sequence found within the original polynucleotide sequence in a biological sample. In embodiments, the endogenous region includes a gene or a fragment thereof. In embodiments, the first endogenous region includes a gene or a fragment thereof. In embodiments, the second endogenous region includes a gene or a fragment thereof. In embodiments, both the first endogenous region and the second endogenous region include a gene or a fragment thereof. In embodiments, the endogenous region includes mRNA. In embodiments, the endogenous region includes genomic DNA (e.g., exons, single nucleotide polymorphisms, mutable regions and/or highly conserved regions). In embodiments, the endogenous region includes a genetic locus. In embodiments, the endogenous region includes autosomal DNA and/or mitochondrial DNA. In embodiments, the endogenous region includes a portion of a gene, a regulatory sequence, genomic DNA, cDNA, RNA including mRNA, miRNA, or rRNA. In embodiments, an endogenous region may refer to the post hoc ligation (i.e., ligation after the fragmentation) of an exogenous sequence to one of the ends of the target polynucleotide. In embodiments, an endogenous region of a target polynucleotide is a sequence of the target polynucleotide present prior to fragmentation. In embodiments, the endogenous region of a target polynucleotide is not an adapter sequence. In embodiments, the endogenous region of a target polynucleotide does not include a universal priming binding sequence. In embodiments, the endogenous region does not include the P5, P7, or complementary sequences thereof (i.e., P5′ or P7′). The P5 and P7 primers are used on the surface of commercial flow cells for sequencing on various Illumina platforms. The P5 and P7 adapter sequences are described in U.S. Patent Publication No. 2011/0059865 A1, which is incorporated herein by reference in its entirety. The terms P5 and P7 may be used when referring to amplification primers, e.g., universal primers. The terms P5′ (P5 prime) and P7′ (P7 prime) refer to the complement of P5 and P7, respectively. In embodiments, an endogenous sequence is not a synthetic sequence. In embodiments, an endogenous sequence is a sequence found in nature. In embodiments, an endogenous sequence is not a synthetic or engineered sequence.

In embodiments, the nucleotides of the present disclosure use a cleavable linker to attach the label to the nucleotide. The use of a cleavable linker ensures that the label can, if required, be removed after detection, avoiding any interfering signal with any labelled nucleotide incorporated subsequently. The use of the term “cleavable linker” is not meant to imply that the whole linker is required to be removed from the nucleotide base. The cleavage site can be located at a position on the linker that ensures that part of the linker remains attached to the nucleotide base after cleavage. The linker can be attached at any position on the nucleotide base provided that Watson-Crick base pairing can still be carried out. In the context of purine bases, it is preferred if the linker is attached via the 7-position of the purine or the preferred deazapurine analogue, via an 8-modified purine, via an N-6 modified adenosine or an N-2 modified guanine. For pyrimidines, attachment is preferably via the 5-position on cytidine, thymidine or uracil and the N-4 position on cytosine.

The term “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd (0), tris-(2-carboxyethyl)phosphine, dilute nitrous acid, fluoride, tris(3-hydroxypropyl)phosphine), sodium dithionite (Na₂S₂O₄), or hydrazine (N₂H₄)). A chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30 °C), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase. A cleavable site may also be referred to herein as a “cleavage domain” or “cleavable domain”.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3®). In embodiments, the cyanine moiety has 5 methine structures (i.e., cyanine 5 or Cy5®). In embodiments, the cyanine moiety has 7 methine structures (i.e., cyanine 7 or Cy7®).

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monocrystalline iron oxide nanoparticles, monocrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In embodiments, a detectable moiety is a moiety (e.g., monovalent form) of a detectable agent.

The terms “fluorophore” or “fluorescent agent” or “fluorescent dye” are used interchangeably and refer to a substance, compound, agent (e.g., a detectable agent), or composition (e.g., compound) that can absorb light at one or more wavelengths and re-emit light at one or more longer wavelengths, relative to the one or more wavelengths of absorbed light. Examples of fluorophores that may be included in the compounds and compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5, DRAQ7, or CyTRAK Orange), pyrene derivatives (e.g., cascade blue and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF dye™, DRAQ™, CyTRAK™, BODIPY™, Alexa Fluor™, DyLight Fluor™, Atto™, Tracy™, FluoProbes™, Abberior Dyes™, DY™ dyes, MegaStokes Dyes™, Sulfo Cy™, Seta™ dyes, SeTau™ dyes, Square Dyes™, Quasar™ dyes, Cal Fluor™ dyes, SureLight Dyes™, PerCP™, Phycobilisomes™, APC™, APCXL™, RPE™, and/or BPE™. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners). In embodiments, the fluorophore is an aromatic (e.g., polyaromatic) moiety having a conjugated π-electron system. In embodiments, the fluorophore is a fluorescent dye moiety, that is, a monovalent fluorophore.

The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group-OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

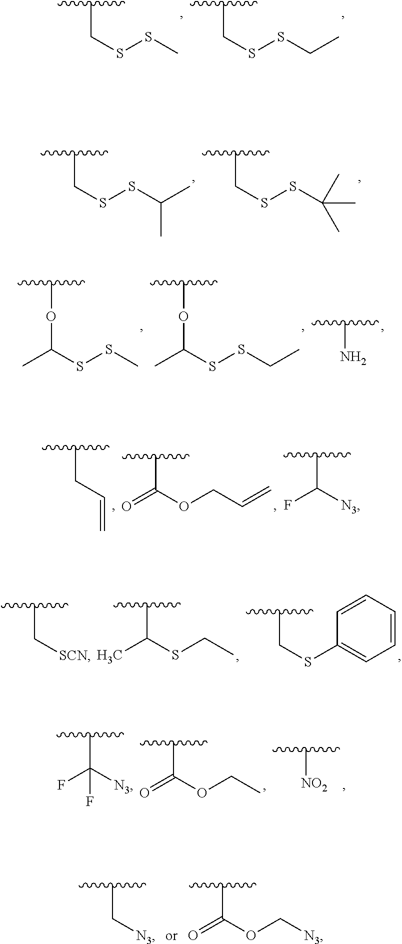

wherein the 3′ oxygen of the nucleotide is not shown in the formulae above. The term “allyl” as described herein refers to an unsubstituted methylene attached to a vinyl group (i.e., —CH—CH₂). In embodiments, the reversible terminator moiety is

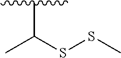 [SMILES: *C(C)SSC]

as described in U.S. Pat. No. 10,738,072, which is incorporated herein by reference for all purposes. For example, a nucleotide including a reversible terminator moiety may be represented by the formula:

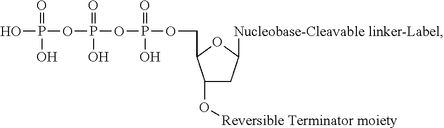

where the nucleobase is adenine or adenine analogue, thymine or thymine analogue, guanine or guanine analogue, or cytosine or cytosine analogue.

In some embodiments, a nucleic acid (e.g., an immobilized oligonucleotide) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

As used herein, the term “biomolecule” refers to an agent (e.g., a compound, macromolecule, or small molecule), and the like derived from a biological system (e.g., an organism). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). In embodiments, a biomolecule may be referred to as an analyte. Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In embodiments, the analytes within a cell can be localized to subcellular locations, including, for example, organelles, e.g., mitochondria, Golgi apparatus, endoplasmic reticulum, chloroplasts, endocytic vesicles, exocytic vesicles, vacuoles, lysosomes, etc. In embodiments, analyte(s) can be peptides or proteins, including antibodies and/or enzymes. In embodiments, an analyte can be detected indirectly, such as through detection of an intermediate agent, for example, a ligation product or an analyte capture agent (e.g., an oligonucleotide-conjugated antibody), such as those described herein.

As used herein, the term “cancer-associated sequence” or “cancer-associated gene” refers to a nucleic acid sequence associated with an altered risk for a cancer (e.g. breast cancer, bladder cancer, or colorectal cancer) or an altered prognosis for a cancer. Exemplary cancer-associated sequences that promote cancer include oncogenes; genes that enhance cell proliferation, invasion, or metastasis; genes that inhibit apoptosis; and pro-angiogenesis genes. Cancer-associated genes that inhibit cancer include, but are not limited to, tumor suppressor genes; genes that inhibit cell proliferation, invasion, or metastasis; genes that promote apoptosis; and anti-angiogenesis genes. A cancer-associated gene, also referred to herein as an oncogene or tumor suppressor gene, is a gene that plays a role in the development of cancer. Oncogenes are genes that, when mutated or overexpressed, can promote cell growth and division, leading to the formation of tumors. Tumor suppressor genes, on the other hand, are genes that normally prevent cells from becoming cancerous, and their loss or inactivation can contribute to the development of cancer. For example, p53 gene is a cancer-associated gene which encodes a tumor suppressor protein, and is frequently found to be mutated in human cancers, and its loss of function can lead to the formation of tumors.

As used herein, the term “cancer-associated genomic loci” refers to any genomic loci determined to be useful for monitoring or detecting a cancer in a patient. The cancer associated genomic loci may be associated with (i) the metastatic potential of the cancer, potential to metastasize to specific organs, risk of recurrence, and/or course of the tumor; (ii) the tumor stage; (iii) the patient prognosis in the absence of treatment of the cancer; (iv) the prognosis of patient response (e.g., tumor shrinkage or progression-free survival) to treatment (e.g., chemotherapy, radiation therapy, surgery to excise tumor, etc.); (v) diagnosis of actual patient response to current and/or past treatment; (vi) determining a preferred course of treatment for the patient; (vii) prognosis for patient relapse after treatment (either treatment in general or some particular treatment); (viii) prognosis of patient life expectancy (e.g., prognosis for overall survival), etc.

As used herein, the term “biological system” refers to a virus, cell, cell derivative, cell nucleus, cell organelle, cell constituent and the like derived from a biological sample. Examples of a cell organelle include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological system (e.g., an organism) may contain multiple individual components, such as viruses, cells, cell derivatives, cell nuclei, cell organelles and cell constituents, including combinations of different of these and other components. The biological system may include DNA, RNA, organelles, proteins, or any combination thereof. These components may be extracellular. In some examples, the biological system may be referred to as a clump or aggregate of combinations of components. In some instances, the biological system may include one or more constituents of a cell but may not include other constituents of the cell. An example of such constituents include nucleus or an organelle. A cell may be a live or viable cell. The live cell may be capable of being cultured, for example, being cultured when enclosed in a gel or polymer matrix or cultured when including a gel or polymer matrix. A biological system may include a single cell and/or a single nuclei from a cell.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9°N polymerase or a variant thereof, E. Coli DNA polymerase I, Bacteriophage T4 DNA polymerase, Sequenase, Taq DNA polymerase, DNA polymerase from Bacillus stearothermophilus, Bst 2.0 DNA polymerase, 9° N polymerase (exo-) A485L/Y409V, Phi29 DNA Polymerase (q29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator™ γ, 9°N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. In the context of selective sequencing, “target polynucleotide(s)” refers to the subset of polynucleotide(s) to be sequenced from within a starting population of polynucleotides.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

As used herein, the terms “specific”, “specifically”, “specificity”, or the like of a compound refers to the compound's ability to cause a particular action, such as binding, to a particular molecular target with minimal or no action to other proteins in the cell.

As used herein, the terms “bind” and “bound” are used in accordance with their plain and ordinary meanings and refer to an association between atoms or molecules. The association can be direct or indirect. For example, bound atoms or molecules may be directly bound to one another, e.g., by a covalent bond or non-covalent bond (e.g., electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex. As used herein, the term “attached” refers to the state of two things being joined, fastened, adhered, connected or bound to each other. For example, a sample such as a cell or tissue, can be attached to a material, such as a hydrogel, polymer, or solid support, by a covalent or non-covalent bond. In embodiments, attachment is a covalent attachment.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support. In embodiments, the solid substrate is in the form of a chip, a bead, a well, a capillary tube, a slide, a wafer, a filter, a fiber, a porous media, or flow cell, or a column. In embodiments, the solid substrate is gold, quartz, silica, plastic, glass, diamond, silver, metal, or polypropylene. In embodiments, the solid substrate is porous.

As used herein, the terms “solid support” and “substrate” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. Solid supports may be in the form of discrete particles, which alone does not imply or require any particular shape. The term “particle” means a small body made of a rigid or semi-rigid material. The body can have a shape characterized, for example, as a sphere, oval, microsphere, or other recognized particle shape whether having regular or irregular dimensions. As used herein, the term “discrete particles” refers to physically distinct particles having discernible boundaries. The term “particle” does not indicate any particular shape. The shapes and sizes of a collection of particles may be different or about the same (e.g., within a desired range of dimensions, or having a desired average or minimum dimension). A particle may be substantially spherical (e.g., microspheres) or have a non-spherical or irregular shape, such as cubic, cuboid, pyramidal, cylindrical, conical, oblong, or disc-shaped, and the like. In embodiments, the particle has the shape of a sphere, cylinder, spherocylinder, or ellipsoid. Discrete particles collected in a container and contacting one another will define a bulk volume containing the particles, and will typically leave some internal fraction of that bulk volume unoccupied by the particles, even when packed closely together. In embodiments, cores and/or core-shell particles are approximately spherical. As used herein the term “spherical” refers to structures which appear substantially or generally of spherical shape to the human eye, and does not require a sphere to a mathematical standard. In other words, “spherical” cores or particles are generally spheroidal in the sense of resembling or approximating to a sphere. In embodiments, the diameter of a spherical core or particle is substantially uniform, e.g., about the same at any point, but may contain imperfections, such as deviations of up to 1, 2, 3, 4, 5 or up to 10%. Because cores or particles may deviate from a perfect sphere, the term “diameter” refers to the longest dimension of a given core or particle. Likewise, polymer shells are not necessarily of perfect uniform thickness all around a given core. Thus, the term “thickness” in relation to a polymer structure (e.g., a shell polymer of a core-shell particle) refers to the average thickness of the polymer layer.

A solid support may further include a polymer or hydrogel on the surface to which the primers are attached (e.g., the primers are covalently attached to the polymer, wherein the polymer is in direct contact with the solid support). Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor®, silica or silica-based materials including silicon and modified silicon, carbon, metals, inorganic glasses, optical fiber bundles, photopatternable dry film resists, UV-cured adhesives and polymers. The solid supports for some embodiments have at least one surface located within a flow cell. The solid support, or regions thereof, can be substantially flat. The solid support can have surface features such as wells, pits, channels, ridges, raised regions, pegs, posts or the like. The term solid support is encompassing of a substrate (e.g., a flow cell) having a surface including a polymer coating covalently attached thereto. In embodiments, the solid support is a flow cell.

Broadly speaking, for nucleic acid sequencing applications, a flow cell may be considered a reaction chamber that contains one or more nucleic acid templates tethered to a solid support, to which nucleotides and ancillary reagents are iteratively applied and washed away. The flow cell allows for imaging of the sites at which the nucleic acids are bound, and resulting image data is used for the desired analysis. The latest commercial sequencing instruments use flow cells and massive parallelization to increase sequencing capacity.

In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. A microfluidic flow channel is characterized by cross-sectional dimensions less than 1000 microns. Usually at least one, and preferably all, cross-sectional dimensions are greater than 500 microns.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

Polymers can be hydrophilic, hydrophobic, or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. In some embodiments, the hydrogel polymer includes 60-90% fluid, such as water, and 10-30% polymer. In certain embodiments, the water content of hydrogel is about 70-80%.

Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO—PPO-PEO copolymers (Pluronics), poly(phosphazene), poly(methacrylates), poly(N-vinylpyrrolidone), PL (G) A-PEO-PL (G) A copolymers, poly(ethylene imine), polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl) cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

The term “array” as used herein, refers to a container (e.g., a microplate, tube, or flow cell) including a plurality of features (e.g., wells). For example, an array may include a container with a plurality of wells. In embodiments, the array is a microplate. In embodiments, the array is a flow cell.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “nanowell” refers to a discrete concave feature or depression in a substrate having a surface opening that is completely surrounded by interstitial region(s) of the surface, wherein the diameter of the feature is less than or equal to 1000 nanometers. In embodiments, the nanowell is within a well (e.g., a well as described herein).

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 500, 600, 700, 800, 900, 1,000, 1,100, 1,200, 1,300, 1,400, 1,500, or more nucleotide bases. In embodiments, a sequencing read is a string of characters representing the sequence of nucleotides. In embodiments, the length of a sequencing read corresponds to the length of the target sequence. In embodiments, the length of a sequencing read corresponds to the number of sequencing cycles. A sequencing read may be subjected to initial processing (often termed “pre-processing”) prior to annotation. Pre-processing includes filtering out low-quality sequences, sequence trimming to remove continuous low-quality nucleotides, merging paired-end sequences, or identifying and filtering out PCR repeats using known techniques in the art. The sequenced reads may then be assembled and aligned using bioinformatic algorithms known in the art. A sequencing read may be aligned to a reference sequence. In embodiments, a sequencing read includes a computationally derived string corresponding to the detected complementary nucleotide (e.g., a labeled nucleotide). The sequence reads are optionally stored in an appropriate data structure for further evaluation. In embodiments, a first sequencing reaction can generate a first sequencing read. The first sequencing read can provide the sequence of a first region of the polynucleotide fragment. In some embodiments, the nucleic acid template is optionally subjected to one or more additional rounds of sequencing using additional sequencing primers, thereby generating additional sequencing reads. In embodiments, generating a sequencing read includes reading a barcode and a template nucleotide sequence. In embodiments, generating a sequencing read includes reading a template nucleotide sequence. In embodiments, generating a sequencing read includes reading a barcode and not a template nucleotide sequence. In embodiments, a sequencing read is about 25 nucleotide bases. In embodiments, a sequencing read is about 35 nucleotide bases. In embodiments, a sequencing read is about 45 nucleotide bases. In embodiments, a sequencing read is about 55 nucleotide bases. In embodiments, a sequencing read is about 65 nucleotide bases. In embodiments, a sequencing read is about 75 nucleotide bases. In embodiments, a sequencing read is about 85 nucleotide bases.

A “gene” refers to a polynucleotide sequence that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5 kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSFIR, ERBA, ERBB, EBRB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WT1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

The term “genetic locus,” or “locus” as used herein refers to a genome or target polynucleotide, specifically a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

As used herein, the terms “incubate,” and “incubation” refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15 °C to 95 °C. In some embodiments, the hybridization is performed at a temperature of about 20 °C, about 25 °C, about 30 °C, about 35 °C, about 40 °C, about 45 °C, about 50 °C, about 55 °C, about 60 °C, about 65 °C, about 70 °C, about 75 °C, about 80 °C, about 85 °C, about 90 °C, or about 95 °C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

A nucleic acid can be amplified by a suitable method. The term “amplification,” “amplified,” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction includes a suitable thermal stable polymerase. Thermal stable polymerases are known in the art and are stable for prolonged periods of time, at temperature greater than 80 °C when compared to common polymerases found in most mammals. In certain embodiments the term “amplification,” “amplified,” or “amplifying” refers to a method that includes a polymerase chain reaction (PCR). Conditions conducive to amplification (i.e., amplification conditions) are well known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits.

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or |
| | | anhydrides |
| diazoalkanes | carboxylic acids | esters |
| epoxides | thiols | thioethers |
| haloacetamides | thiols | thioethers |
| haloplatinate | amino | platinum |
| | | complex |
| haloplatinate | heterocycle | platinum |
| | | complex |
| haloplatinate | thiol | platinum |
| | | complex |
| halotriazines | amines/anilines | aminotriazines |
| halotriazines | alcohols/phenols | triazinyl |
| | | ethers |
| halotriazines | thiols | triazinyl |
| | | thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite |
| | | esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate |
| | | esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals 10 interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine). Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds.; (n) azides coupled to alkynes using copper catalyzed cycloaddition click chemistry; (o) biotin conjugate can react with avidin or strepavidin to form a avidin-biotin complex or streptavidin-biotin complex.

The term “covalent linker” is used in accordance with its ordinary meaning and refers to a divalent moiety which connects at least two moieties to form a molecule. The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

The term “adapter” as used herein refers to any synthetic oligonucleotide that can be attached (e.g., by ligation, hybridization, or transposition) to a nucleic acid molecule, thereby generating nucleic acid products capable of being sequenced on a sequencing platform (e.g., an Illumina® or Singular Genomics® sequencing platform). In embodiments, the adapter includes a platform primer binding sequence, rendering the nucleic acid molecule capable of hybridizing to and amplifying on a solid support. In embodiments, adapter(s) include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In embodiments, the adapter includes a synthetic sequence (e.g., optimized for a particular property, such as a melting temperature and/or the percentage of guanine and cytosines. In embodiments, an adapter is an engineered sequence that typically include sites for restriction endonuclease recognition and/or cutting and sites for primer binding (e.g., for amplifying or sequencing the library constructs).

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation. A “tissue section” as used herein refers to a portion of a biological tissue derived from a biological sample, typically from an organism (e.g., a human or animal subject or patient).

As used herein, the term “fresh,” generally in the context of a fresh tissue means that the tissue has recently been obtained from an organism, generally before any subsequent fixation steps, for example, flash freezing or chemical fixation. In embodiments, a fresh tissue is obtained from an organism about 1 second up to about 20 minutes before any fixation steps are performed. In embodiments, a fresh tissue is obtained from an organism about 1 second up to about 60 seconds before any fixation steps are performed. In embodiments, a fresh tissue is obtained from an organism about 30 seconds up to about 60 seconds before any fixation steps are performed. In embodiments, a fresh tissue is obtained from an organism about 1 minutes up to about 20 minutes before any fixation steps are performed. In embodiments, a fresh tissue is obtained from an organism about 1 minutes up to about 10 minutes before any fixation steps are performed. In embodiments, a fresh tissue is obtained from an organism about 1 minutes up to about 5 minutes before any fixation steps are performed. In embodiments, a fresh tissue is obtained from an organism about 30 seconds, about 1 minute, about 2 minutes, about 3 minutes, about 4 minutes, about 5 minutes, about 10 minutes, about 15 minutes, or about 20 minutes before any fixation steps are performed.

As used herein, the term “fix,” refers to formation of covalent bonds, such as crosslinks, between biomolecules or within molecules. The process of fixing tissue samples or biological samples (e.g., cells and nuclei) for example, is called “fixation.” The agent that causes fixation is generally referred to as a “fixative” or “fixing agent.” “Fixed biological samples” (e.g., fixed cells or nuclei) or “fixed tissues” refers to biological samples (e.g., cells or nuclei) or tissues that have been in contact with a fixative under conditions sufficient to allow or result in formation of intra- and inter-molecular crosslinks between biomolecules in the biological sample. Fixation may be reversed and the process of reversing fixation may be referred to as “un-fixing” or “decrosslinking.” Unfixing or decrosslinking refers to breaking or reversing the formation of covalent bonds in biomolecules formed by fixatives. In some examples, the tissue fixed is fresh tissue. In some examples, the tissue fixed may be frozen tissue. In some examples, the tissue fixed may not be dissociated. In some examples, the tissue fixed may be dissociated or partially dissociated (e.g., chopped, cut). In some examples, tissue that has been rapidly frozen and, perhaps, cut or chopped into pieces (e.g., small enough to fit into a tube or container used for fixation) may be used. In some examples, tissue may be dissociated or partially dissociated (e.g., cut, chopped) before or during fixation. In some examples, tissue that is fixed may not be dissociated. The frozen biological tissue can be fixed using a fixing agent, which is suitably an organic fixing agent. Suitable organic fixing agents include without limitation alcohols, ketones, aldehydes (e.g., glutaraldehyde), cross-linking agents, disuccinimidyl suberate (DSS), dimethylsuberimidate (DMS), formalin, dimethyladipimidate (DMA), dithio-bis(-succinimidyl propionate) (DSP), disuccinimidyl tartrate (DST), ethylene glycol bis(succinimidyl succinate) (EGS), bis(sulfosuccinimidyl) suberate (BS3) and combinations thereof. A particularly suitable fixing agent is a formaldehyde-based fixing agent such as formalin, which is a mixture of formaldehyde and water. The formalin may include about 1% to about 15% by weight formaldehyde and about 85% to about 99% by weight water, suitable about 2% to about 8% by weight formaldehyde and about 92% to about 98% by weight water, or about 4% by weight formaldehyde and about 96% by weight water. In some examples, tissues may be fixed in 4% paraformaldehyde. Other suitable fixing agents will be appreciated by those of ordinary skill in the art (e.g., International PCT App. No. PCT/US2020/066705, which is incorporated herein by reference in its entirety).

As used herein, the term “permeable” refers to a property of a substance that allows certain materials to pass through the substance. “Permeable” may be used to describe a biological sample, such as a cell or nucleus, in which analytes in the biological sample can leave the biological sample. “Permeabilize” is an action taken to cause, for example, a biological sample (e.g., a cell) to release its analytes. In some examples, permeabilization of a biological sample is accomplished by affecting the integrity (e.g., compromising) of a biological sample membrane (e.g., a cellular or nuclear membrane) such as by application of a protease or other enzyme capable of disturbing a membrane allowing analytes to diffuse out of the biological sample.

As used herein, the term “single biological sample”, such as a single cell or a single nucleus generally refers to a biological sample that is not present in an aggregated form or clump. Single biological samples, such as cells and/or nuclei may be the result of dissociating a tissue sample.

As used herein, the term “disease state” is used in accordance with its plain and ordinary meaning and refers to any abnormal biological or aberrant state of a cell or organism. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

As used herein, the term “tissue” is used in accordance with its plain and ordinary meaning and refers to an organization of cells in a structure, where the structure generally functions as a unit in an organism (e.g., mammals) and may carry out specific functions. In some examples, cells in a tissue are configured in a mass and may not be free from one another. This disclosure describes methods of obtaining single biological samples (e.g., cells or nuclei) from tissues that can be used in various single biological samples (e.g., single-cell/nucleus) workflows. In some examples, blood cells (e.g., lymphocytes) can be considered a tissue. However, blood cells, like lymphocytes, generally are free from one another in the blood. The methods disclosed herein can be used to process those cells to obtain cells and/or nuclei, although dissociation steps may not be necessary when using those types of tissues. Generally, any type of tissue can be used in the methods described herein. Examples of tissues that may be used in the disclosed methods include, but are not limited to connective, epithelial, muscle and nervous tissue. In some examples, the tissues are from mammals. Tissues that contain any type of cells may be used. For example, tissues from abdomen, bladder, brain, esophagus, heart, intestine, kidney, liver, lung, lymph node, olfactory bulb, ovary, pancreas, skin, spleen, stomach, testicle, and the like. The tissue may be normal or tumor tissue (e.g., malignant). This example is not meant to be limiting. Although the conditions used in the disclosed may not be identical for different types of tissue, the methods may be applied to any tissue. The tissues used in the disclosed methods may be in various states. In some examples, the tissues used in the disclosed methods may be fresh, frozen, or fixed.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes. In embodiments, a cellular component is a biomolecule.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics™ (e.g., the G4™ system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri (carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

As used herein, a “plurality” refers to two or more.

As used herein the terms “automated” and “semi-automated” mean that the operations are performed by system programming or configuration with little or no human interaction once the operations are initiated, or once processes including the operations are initiated.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2-dimensional area defined by straight line axes x and y. When used in reference to a detecting apparatus and an object observed by the detector, the xy plane may be specified as being orthogonal to the direction of observation between the detector and object being detected.

“Synthetic” agents refer to non-naturally occurring agents, such as enzymes or nucleotides derived or constructed using human-made techniques. For example, s synthetic DNA polymerases refer to non-naturally occurring DNA polymerases such as those constructed by synthetic methods, mutated parent DNA polymerases such as truncated DNA polymerases and fusion DNA polymerases. Synthetic oligonucleotides such as adapter sequences or primers, include a human-designed sequence, typically configured to maximize yield and minimize off-target products, without introducing any biases. Examples of synthetic oligonucleotide sequences include, for example, sequences used for cluster generation and sequencing on a sequencing device (e.g., the G4™ sequencing platform) include SP2′: 5′-GATCGGAAGAGCACACGTCTGAACTCCAGTC (SEQ ID NO:1), S2′: 5′-AGTGGTTGGTGAGGGTCATCTCGCTGGAG (SEQ ID NO:2), S1: 5′-ACAAAGGCAGCCACGCACTCCTTCCCTGT (SEQ ID NO:3), and SP1: ACACTCTTTCCCTACACGACGCTCTTCCGATCT (SEQ ID NO:4), or a complement thereof. Additional examples of synthetic oligonucleotide sequences include P5, P7, or complementary sequences thereof (i.e., P5′ or P7′). The P5 and P7 primers (e.g., P5: 5′-AATGATACGGCGACCACCG (SEQ ID NO: 5), or the complement thereof, and P7: 5′-CAAGCAGAAGACGGCATACGA (SEQ ID NO: 6), or the complement thereof) are used on the surface of commercial flow cells for sequencing on various Illumina platforms, as described in U.S. Patent Publication No. 2011/0059865 A1.

The term “library” merely refers to a collection or plurality of template nucleic acid molecules which share common sequences at their 5′ ends (e.g., the first end) and common sequences at their 3′ ends (e.g., the second end). In embodiments, a population of template nucleic acid molecules form a library.

### II. METHODS

In an aspect is a method of immobilizing a polynucleotide to a solid support. In embodiments, the method includes binding a polynucleotide to a solid support. In embodiments, the method includes introducing a target polynucleotide into a reaction vessel (e.g., a flow cell) containing a solid support with immobilized oligonucleotides. In embodiments, the target polynucleotide does not contain pre-attached functional sequences required for hybridization to the solid support. In embodiments, solution-phase primers are introduced into the reaction vessel, wherein the primers include target-binding sequences and functional sequences that enable downstream hybridization to the solid support and amplification. In embodiments, the primers hybridize to the target polynucleotide and are extended by a polymerase to generate a modified polynucleotide incorporating the functional sequences. In embodiments, the modified polynucleotide hybridizes to the immobilized oligonucleotides on the solid support, wherein the immobilized oligonucleotides contain either a capture sequence complementary to one of the functional sequences or serve as a splint to facilitate circularization of the target polynucleotide. In embodiments, upon hybridization, the polynucleotide undergoes amplification, including solid-phase amplification or rolling circle amplification (RCA), to generate amplicons for sequencing. In embodiments, the amplified polynucleotides are subsequently detected through sequencing.

In embodiments, the reaction vessel is a flow cell, a microplate, or a multiwell container. In embodiments, the target polynucleotide comprises a first endogenous region and a second endogenous region. In embodiments, the first target binding sequence is capable of hybridizing to the first endogenous region of the target polynucleotide. In embodiments, the second target binding sequence is capable of hybridizing to a complement of the second endogenous region of the target polynucleotide. In embodiments, the second target binding sequence is substantially identical, for example, greater than 99% identical, to a second endogenous region of the target polynucleotide.

In embodiments, the method includes adding to a reaction vessel a first primer, a second primer, and a target polynucleotide, wherein the reaction vessel includes a solid support and a third primer attached to the solid support, wherein the first primer includes a first target binding sequence and a first platform primer binding sequence, the second primer includes a second target binding sequence and a second platform primer sequence, and the third primer includes a sequence complementary to the second platform primer sequence; and within the reaction vessel: (a) binding the first target binding sequence to the target polynucleotide and extending the first primer to generate a first complement of the target polynucleotide template includes the first platform primer binding sequence and a complementary sequence of the target polynucleotide; (b) binding the second target binding sequence to the first complement of the target polynucleotide and extending the second primer to generate a second template includes a first platform primer sequence, a sequence of the target polynucleotide, and the second platform primer sequence; and (c) binding the second platform primer sequence to the third primer and extending the third primer to form an immobilized polynucleotide. In embodiments, the target polynucleotide is seeded (i.e., immobilized) onto the solid support without any prior adapter ligation step. In embodiments, the target polynucleotide includes a first endogenous region and a second endogenous region. In embodiments, the first target binding sequence is capable of binding to the first endogenous region of the target polynucleotide. In embodiments, the second target binding sequence is capable of binding to complement of the second endogenous region of the target polynucleotide. In embodiments, the second target binding sequence is substantially identical (e.g., greater than 99% identical) to a second endogenous region of the target polynucleotide. In embodiments, the reaction vessel is a flow cell. In embodiments, the reaction vessel is a microplate. In embodiments, the reaction vessel is a multiwell container.

In embodiments, the method includes forming a circular polynucleotide including a complement of a sequence of the target polynucleotide. In embodiments, the method includes adding to a flow cell a target polynucleotide and a circularizable polynucleotide, wherein the circularizable polynucleotide includes a platform primer binding sequence (i.e., a sequence complementary to a primer oligonucleotide attached to the solid support). In embodiments, the circularizable polynucleotide binds to a first sequence of the target polynucleotide and a second sequence of the target polynucleotide, wherein a gap sequence is between the first and second sequences. In embodiments, a polymerase extends the circularizable polynucleotide to form a complement of the gap sequence, and is circularized (e.g., ligated with a ligase enzyme) to form a circular polynucleotide. The circular polynucleotide may bind to the immobilized platform primer via the platform primer binding sequence, and in the presence of a strand-displacing polymerase, the immobilized platform primer is extended to generate multiple copies of the gap sequence. In embodiments, the gap sequence is 1 to 200 nucleotides. In embodiments, the gap sequence is an unknown sequence. In embodiments, the gap sequence is 5, 10, 15, 20, 25 or more nucleotides. In embodiments, the gap sequence is 20 to 80 nucleotides. In embodiments, the method further includes amplifying the circular oligonucleotide by extending an amplification primer with a polymerase (e.g., a strand-displacing polymerase), wherein the primer extension generates an extension product including multiple complements of the circular oligonucleotide, referred to as an amplicon. An amplicon typically contains multiple, tandem copies of the circularized nucleic acid molecule of the corresponding sample nucleic acid. The number of copies can be varied by appropriate modification of the reaction conditions, such as varying the number of amplification cycles, using polymerases of varying processivity in the amplification reaction, or varying the length of time that the amplification reaction is run. In embodiments, the extension product includes three or more copies of the circular oligonucleotide. In embodiments, the circular oligonucleotide is copied about 3-50 times (i.e., the extension product includes about 3 to 50 complements of the circular oligonucleotide). In embodiments, the circular oligonucleotide is copied about 50-100 times (i.e., the extension product includes about 50 to 100 complements of the circular oligonucleotide). In embodiments, the circular oligonucleotide is copied about 100-300 times (i.e., the extension product includes about 100 to 300 complements of the circular oligonucleotide). In embodiments, the method includes hybridizing an amplification primer and oligonucleotide primer in the same reaction (e.g., simultaneously). In embodiments, the oligonucleotide is extended as an amplification primer after generating the circular oligonucleotide (e.g., the 3′ end of the oligonucleotide hybridized to the circular oligonucleotide is extended with a polymerase). In embodiments, the first primer and second primer are linked via a polynucleotide backbone and may be referred to as a circularizable polynucleotide.

In embodiments, the circularizable polynucleotide includes a first hybridization sequence capable of hybridizing to a first sequence of a target polynucleotide (e.g., a first target binding sequence), a second hybridization sequence capable of hybridizing to a second sequence of the target polynucleotide (e.g., a second target binding sequence). In embodiments, the circularizable polynucleotide includes a barcode sequence. In embodiments, the circularizable probe (e.g., the circularizable polynucleotide) includes a 5′ end and a 3′ end, wherein a first region at the 5′ end is complementary to a first sequence of a target polynucleotide, and wherein a second region at the 3′ end is complementary to a second sequence of the target polynucleotide. In embodiments, the first sequence and the second sequence of the target polynucleotide are adjacent to each other. In embodiments, the first sequence and the second sequence of the target polynucleotide are separated by 1 or more nucleotides. In embodiments, the first sequence and the second sequence of the target polynucleotide are separated by 1, 5, 10, 20, 30, 40, 50, 75, 100, or more nucleotides. In embodiments, the first sequence and the second sequence of the target polynucleotide flank a target sequence. In embodiments, the target sequence is a barcode sequence. In embodiments, the gap sequence is 5, 10, 15, 20, 25, 30, 40, 50, 60, 70, 80, 90, 100, 125, 150, 175, or 200 nucleotides. In embodiments, the gap sequence is 5 to 150 nucleotides. In embodiments, the gap sequence is 1, 2, 3, 4, or 5 nucleotides.

In embodiments, the method further includes ligating the 5′ and 3′ ends of the circularizable polynucleotide described herein or the polynucleotide probe described herein to form a circular polynucleotide (i.e., a polynucleotide that is a continuous strand lacking free 5′ and 3′ ends). In embodiments, the method includes ligating the 5′ and 3′ ends of the polynucleotide to form a circular polynucleotide, wherein the circular polynucleotide includes the target nucleic acid. In embodiments, the method includes ligating the 5′ and 3′ ends of the polynucleotide to form a circular polynucleotide, wherein the circular polynucleotide includes the oligonucleotide barcode. In embodiments, the ligation includes enzymatic ligation. In embodiments, ligating includes enzymatic ligation including a ligation enzyme (e.g., CircLigase™ enzyme, Taq DNA Ligase, HiFi Taq DNA Ligase, T4 ligase, PBCV-1 DNA Ligase (also known as SplintR® ligase) or Ampligase™ DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR® ligase) or a Taq DNA Ligase. In embodiments, the ligase enzyme is a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR® ligase), and combinations thereof. In embodiments, prior to ligating the 5′ and 3′ ends of the polynucleotide probe to form a circular polynucleotide, the method includes extending the 3′ end of the polynucleotide probe (using a polymerase to incorporate one or more nucleotides) along the oligonucleotide to generate a complementary sequence and ligating the extended 3′ end of the polynucleotide probe to the 5′ end of the polynucleotide probe.

In embodiments, binding includes incubation in a buffer at 40 °C to 50 °C. In embodiments, binding includes incubation in a buffer at 50 °C to 60 °C. In embodiments, binding includes incubation in a buffer at about 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, 50 °C, 51 °C, 52 °C, 53 °C, 54 °C, 55 °C, 56 °C, 57 °C, 58 °C, 59 °C, 60 °C, 61 °C, 62 °C, 63 °C, 64 °C, or 65 °C.

In embodiments, a) occurs for about 1 minute, about 2 minutes, about 3 minutes, about 4 minutes, about 5 minutes, about 6 minutes, about 7 minutes, about 8 minutes, about 9 minutes, or about 10 minutes. In embodiments, a) occurs for less than 1 minute. In embodiments, a) occurs for about 1 minute. In embodiments, a) occurs for about 2 minutes. In embodiments, a) occurs for about 3 minutes. In embodiments, a) occurs for about 4 minutes. In embodiments, a) occurs for about 5 minutes. In embodiments, a) occurs for about 6 minutes. In embodiments, a) occurs for about 7 minutes. In embodiments, a) occurs for about 8 minutes. In embodiments, a) occurs for about 9 minutes. In embodiments, a) occurs for about 10 minutes. In embodiments, a) occurs for about 11 minutes. In embodiments, a) occurs for about 12 minutes. In embodiments, a) occurs for about 13 minutes. In embodiments, a) occurs for about 14 minutes. In embodiments, a) occurs for about 15 minutes. In embodiments, a) occurs for about 16 minutes. In embodiments, a) occurs for about 17 minutes. In embodiments, a) occurs for about 18 minutes. In embodiments, a) occurs for about 19 minutes. In embodiments, a) occurs for about 20 minutes.

In embodiments, a) includes binding the 3′ end of the first target binding sequence to the target polynucleotide. In embodiments, a) includes binding the 3′ end of the first endogenous region of the target polynucleotide.

In embodiments, after a) the first complement includes, from 5′ to 3′, the first platform primer binding sequence and a complementary sequence of the target polynucleotide. In embodiments, after a) the first complement includes, from 5′ to 3′, the first platform primer binding sequence and a complementary sequence of the target polynucleotide, wherein the complementary sequence of the target polynucleotide includes a complementary sequence of the first endogenous region and the second endogenous region.

In embodiments, b) occurs for about 1 minute, about 2 minutes, about 3 minutes, about 4 minutes, about 5 minutes, about 6 minutes, about 7 minutes, about 8 minutes, about 9 minutes, or about 10 minutes. In embodiments, b) occurs for less than 1 minute. In embodiments, b) occurs for about 1 minute. In embodiments, b) occurs for about 2 minutes. In embodiments, b) occurs for about 3 minutes. In embodiments, b) occurs for about 4 minutes. In embodiments, b) occurs for about 5 minutes. In embodiments, b) occurs for about 6 minutes. In embodiments, b) occurs for about 7 minutes. In embodiments, b) occurs for about 8 minutes. In embodiments, b) occurs for about 9 minutes. In embodiments, b) occurs for about 10 minutes. In embodiments, b) occurs for about 11 minutes. In embodiments, b) occurs for about 12 minutes. In embodiments, b) occurs for about 13 minutes. In embodiments, b) occurs for about 14 minutes. In embodiments, b) occurs for about 15 minutes. In embodiments, b) occurs for about 16 minutes. In embodiments, b) occurs for about 17 minutes. In embodiments, b) occurs for about 18 minutes. In embodiments, b) occurs for about 19 minutes. In embodiments, b) occurs for about 20 minutes.

In embodiments, b) includes binding the 3′ end of the second target binding sequence to the target polynucleotide. In embodiments, a) includes binding the 3′ end of the complement of the second endogenous region of the target polynucleotide.

In embodiments, after b) the second template includes, from 5′ to 3′, the first platform primer binding sequence and a sequence of the target polynucleotide. In embodiments, after b) the first complement includes, from 5′ to 3′, the first platform primer binding sequence and the sequence of the target polynucleotide, wherein the sequence of the target polynucleotide includes a sequence of the first endogenous region and the second endogenous region.

In embodiments, the method further includes d) annealing the immobilized polynucleotide to the fourth primer; and e) extending the fourth primer to generate a complement of the immobilized polynucleotide. In embodiments, the method further includes repeating steps a) to e), thereby immobilizing a plurality of target polynucleotides.

In embodiments, extending the first primer to generate a first complement of the target polynucleotide template in step a) includes a polymerase. In embodiments, extending the second primer to generate a second template in step b) includes a polymerase. In embodiments, extending the third primer to form an immobilized polynucleotide includes a polymerase. In embodiments, extending the fourth primer to generate a complement of the immobilized polynucleotide includes a polymerase.

In embodiments, the target polynucleotides do not include a common sequence (e.g., the same sequence within the plurality, otherwise referred to as a universal sequence). In embodiments, the target polynucleotides do not include a synthetic sequence (e.g., a primer binding sequence). In embodiments, the target polynucleotide does not include a universal primer binding sequence (e.g., a polynucleotide sequence that is common to a majority, or substantially all of the target polynucleotides in the library). In embodiments, the target polynucleotide is a fragmented polynucleotide. In embodiments, the target polynucleotide is genomic DNA (gDNA). In embodiments, the target polynucleotide is genomic DNA (gDNA) including a sequence that encodes for a protein. In embodiments, the target polynucleotide is sheared genomic DNA. In embodiments, the target polynucleotide includes a gene sequence.

The target polynucleotide may be double-stranded (dsDNA) or single-stranded (ssDNA). In embodiments, the method includes contacting a sample including one or more target polynucleotides with a denaturant (e.g., NaOH or formamide) to form single-stranded target polynucleotides. In embodiments, the single-stranded target polynucleotides contact the solid support as described herein, and the temperature is increased to greater than 90 °C, followed by a slow reduction in temperature to about 65 °C where the temperature is maintained at about 65 °C for about 30 minutes. Depending on the predicted melting temperature of the first and second oligonucleotides, the annealing temperature may be further adjusted. The ssDNA (i.e., the target polynucleotides) hybridizes to the first oligonucleotide and the remaining unbound polynucleotides are washed away. A polymerase is introduced and extends the first oligonucleotide to form an extension strand containing a complementary sequence of the target polynucleotide attached to the solid support. The original target polynucleotide may be removed, and the temperature is maintained at a sufficient annealing temperature to permit hybridization of the extension strand to the second oligonucleotide. Subsequent extension, denaturation, and annealing is performed to generate a plurality (e.g., a cluster) of immobilized target polynucleotides, and complements thereof.

In embodiments, the target polynucleotide includes a gene or fragment thereof. In embodiments, the target polynucleotide includes an NRAS gene, or a portion thereof, a MLF2 gene, or a portion thereof, a KRAS gene, or a portion thereof, a TP53 gene, or a portion thereof, a ERBB2 gene, or a portion thereof, a IDH1 gene, or a portion thereof, a PIK3CA gene, or a portion thereof, a KIT gene, or a portion thereof, a PIK3R1 gene, or a portion thereof, a NPM1 gene, or a portion thereof, a EGFR gene, or a portion thereof, a BRAF gene, or a portion thereof, or a JAK2 gene, or a portion thereof. In embodiments, the target polynucleotide includes a mutated NRAS gene, or a portion thereof, a mutated MLF2 gene, or a portion thereof, a mutated KRAS gene, or a portion thereof, a mutated TP53 gene, or a portion thereof, a mutated ERBB2 gene, or a portion thereof, a mutated IDH1 gene, or a portion thereof, a mutated PIK3CA gene, or a portion thereof, a mutated KIT gene, or a portion thereof, a mutated PIK3R1 gene, or a portion thereof, a mutated NPM1 gene, or a portion thereof, a mutated EGFR gene, or a portion thereof, a mutated BRAF gene, or a portion thereof, or a mutated JAK2 gene, or a portion thereof. In embodiments, the target polynucleotide encodes for a protein as described herein. In embodiments, the target polynucleotide includes a gene sequence as described herein.

In embodiments, the target polynucleotide is a cancer-associated gene or fragment thereof. In embodiments, the target polynucleotide comprises a gene or fragment thereof that is associated with a rare disease. In embodiments, the target polynucleotide comprises a gene or fragment thereof that is associated with antimicrobial activity. In embodiments, the target polynucleotide comprises a gene or fragment thereof that is associated with antibacterial activity. In embodiments, the target polynucleotide comprises a gene or fragment thereof that is associated with antiviral activity. In embodiments, the target polynucleotide comprises a gene or fragment thereof that is associated with immunomodulatory activity. In embodiments, the target polynucleotide comprises a gene or fragment thereof that is associated with T cells. In embodiments, the target polynucleotide comprises a gene or fragment thereof that is associated with B cells. In embodiments, the target polynucleotide includes a gene encoding a drug metabolizing enzyme. In embodiments, the target polynucleotide includes a gene encoding a drug transporter. In embodiments, the target polynucleotide includes a gene encoding a drug transporter, wherein the drug transporter is a solute carrier protein. In embodiments, the target polynucleotide includes a gene encoding a drug transporter, wherein the drug transporter is an ATP binding cassette family protein.

In embodiments, the target polynucleotide comprises an RNA transcript that is associated with a cancer-associated gene or fragment thereof. In embodiments, the target polynucleotide comprises an RNA transcript that is associated with a rare disease. In embodiments, the target polynucleotide comprises an RNA transcript that is associated with antimicrobial activity. In embodiments, the target polynucleotide comprises an RNA transcript that is associated with antibacterial activity. In embodiments, the target polynucleotide comprises an RNA transcript that is associated with antiviral activity. In embodiments, the target polynucleotide comprises an RNA transcript that is associated with immunomodulatory activity. In embodiments, the target polynucleotide comprises an RNA transcript that is associated with a T cell. In embodiments, the target polynucleotide comprises an RNA transcript that is associated with a B cell. In embodiments, the target polynucleotide includes an RNA transcript encoding a drug metabolizing enzyme. In embodiments, the target polynucleotide transcript includes an RNA transcript encoding a membrane transporter. In embodiments, the target polynucleotide includes an RNA transcript encoding a drug transporter. In embodiments, the target polynucleotide includes an RNA transcript encoding a drug transporter, wherein the drug transporter is a solute carrier protein. In embodiments, the target polynucleotide includes an RNA transcript encoding a drug transporter, wherein the drug transporter is an ATP binding cassette family protein.

In embodiments, the target polynucleotide includes a somatic mutation. In embodiments, the target polynucleotide includes a germline variant. In embodiments, the target polynucleotide includes a single nucleotide polymorphism. In embodiments, the target polynucleotide includes a structural variant. In embodiments, the target polynucleotide includes a structural variant, wherein the structural variant comprises an inversion sequence. In embodiments, the target polynucleotide includes a single nucleotide variant. In embodiments, the target polynucleotide includes a copy number variant. In embodiments, the target polynucleotide includes an insertion or deletion. In embodiments, the target polynucleotide includes a single nucleotide variant and copy number variant. In embodiments, the target polynucleotide includes an insertion or deletion and single nucleotide variant. In embodiments, the target polynucleotide includes a variant sequence in a regulatory or intronic region of the gene. In embodiments, the target polynucleotide includes an intronic variant. In embodiments, the target polynucleotide includes a variant in a non-coding region of the gene. In embodiments, the target polynucleotide includes a repeat expansion. In embodiments, the target polynucleotide includes a trinucleotide repeat expansion.

In embodiments, the target polynucleotide is about 50 nucleotides to about 200 nucleotides in length. In embodiments, the target polynucleotide is about 50, about 60, about 70, about 80, about 90, about 100, about 110, about 120, about 130, about 140, about 150, about 160, about 170, about 180, about 190, or about 200 nucleotides in length. In embodiments, the target polynucleotide is about 60 nucleotides to about 150 nucleotides in length. In embodiments, the target polynucleotide is about 80 nucleotides to about 120 nucleotides in length. In embodiments, the target polynucleotide is about 60, about 80, about 100, about 120, about 140, or about 150 nucleotides in length.

In embodiments, a target polynucleotide sequence is a polynucleotide sequence found within the original polynucleotide sequence in a biological sample. In embodiments, the target polynucleotide sequence includes a gene or a fragment thereof. In embodiments, the first target polynucleotide includes a gene or a fragment thereof. In embodiments, the second target polynucleotide includes a gene or a fragment thereof. In embodiments, both the first target polynucleotide and the second target polynucleotide include a gene or a fragment thereof. In embodiments, the target polynucleotide sequence includes mRNA. In embodiments, the target polynucleotide sequence includes genomic DNA (e.g., exons, single nucleotide polymorphisms, mutable regions and/or highly conserved regions). In embodiments, the target polynucleotide sequence includes a genetic locus. In embodiments, the target polynucleotide sequence includes autosomal DNA and/or mitochondrial DNA. In embodiments, the target polynucleotide sequence includes a portion of a gene, a regulatory sequence, genomic DNA, cDNA, RNA including mRNA, miRNA, or rRNA. In embodiments, the target polynucleotide is a sequence of the target polynucleotide present prior to fragmentation. In embodiments, a target polynucleotide sequence does not include an adapter sequence. In embodiments, a target polynucleotide sequence does not include a universal priming binding sequence. In embodiments, a target polynucleotide sequence does not include the P5, P7, or complementary sequences thereof (i.e., P5′ or P7′). The P5 and P7 primers are used on the surface of commercial flow cells for sequencing on various Illumina® platforms.

In embodiments, the target polynucleotide includes a sequence that complementary to a continuous 20 nucleotide sequence within SEQ ID NO:7, SEQ ID NO:8, SEQ ID NO:9, SEQ ID NO: 10, SEQ ID NO:11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, SEQ ID NO:22, SEQ ID NO:23, SEQ ID NO:24, SEQ ID NO:25, SEQ ID NO:26, SEQ ID NO: 27, SEQ ID NO:28, SEQ ID NO:29, SEQ ID NO:30, SEQ ID NO:31, SEQ ID NO:32, SEQ ID NO: 33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO: 44, SEQ ID NO:45, SEQ ID NO:46, SEQ ID NO:47, SEQ ID NO:48, SEQ ID NO:49, SEQ ID NO: 50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, or SEQ ID NO:60.

In embodiments, the target polynucleotide includes a first endogenous region and a second endogenous region. In embodiments, the target polynucleotide includes a first endogenous region. In embodiments, the target polynucleotide includes a second endogenous region. In embodiments, the first endogenous region is capable of hybridizing with the first target binding sequence in the first primer. In embodiments, the first endogenous region is capable of hybridizing with the second target binding sequence in the second primer. In embodiments, the complement of the first endogenous region is capable of hybridizing with the first target binding sequence in the first primer. In embodiments, the complement of the first endogenous region is capable of hybridizing with the second target binding sequence in the first primer. In embodiments, the second endogenous region is capable of hybridizing with the first target binding sequence in the first primer. In embodiments, the second endogenous region is capable of hybridizing with the second target binding sequence in the second primer. In embodiments, the complement of the second endogenous region is capable of hybridizing with the first target binding sequence in the first primer. In embodiments, the complement of the second endogenous region is capable of hybridizing with the second target binding sequence in the second primer. In embodiments, the first endogenous region is about 10 to 100 nucleotides in length. In embodiments, the first endogenous region is between 10 and 40 nucleotides in length. The length and complexity may vary. In embodiments, the first endogenous region is about 200 nucleotides or less. In embodiments, the first endogenous region is about 10 to 150 nucleotides, 15 to 150 nucleotides, 5 to 100 nucleotides, 5 to 50 nucleotides or 10 to 50 nucleotides. In embodiments, the second endogenous region is about 10 to 100 nucleotides in length. In embodiments, the second endogenous region is between 10 and 40 nucleotides in length. The length and complexity may vary. In embodiments, the second endogenous region is about 200 nucleotides or less. In embodiments, the second endogenous region is about 10 to 150 nucleotides, 15 to 150 nucleotides, 5 to 100 nucleotides, 5 to 50 nucleotides or 10 to 50 nucleotides. The gap between the two regions (i.e., the first endogenous region and the second endogenous region) may be from a few base pairs up to 5,000 base pairs or more. The gap size may be from 10 base pairs to 2,000 base pairs, or 50-500 base pairs. In embodiments, the gap size may be from any combination of 50, 75, 100, 125, 150, 175, 200, or 225 base pairs to 150, 200, 250, 300, 350, 400, 450 or 500 base pairs in length.

In embodiments, the target polynucleotide does not include a universal primer binding sequence. As used herein, the term “universal primer binding sequence” refers to a polynucleotide sequence that is common to two or more nucleic acid molecules where the molecules also have regions of sequence that differ from each other. Non-limiting examples of universal primer binding sites include sequences that are identical to or complementary to P5 and P7 primers, for example, P5: 5′-AATGATACGGCGACCACCG (SEQ ID NO: 5), or the complement thereof, and P7: 5′-CAAGCAGAAGACGGCATACGA (SEQ ID NO: 6), or the complement thereof. In embodiments, the target polynucleotide does not include a P5 sequence, or complement thereof. In embodiments, the target polynucleotide does not include a P7 sequence, or complement thereof. In embodiments, the target polynucleotide does not include a P5 sequence and/or P7 sequence, or complements thereof. In embodiments, the target polynucleotide does not include SEQ ID NO: 5, or a complement thereof. In embodiments, the target polynucleotide does not include SEQ ID NO: 6, or a complement thereof. In embodiments, the target polynucleotide does not include SEQ ID NO: 5 and/or SEQ ID NO: 6, or complements thereof. For example, the target polynucleotide is not ligated to an adapter including a universal primer binding sequence, therefore the target polynucleotide does not include a universal primer binding sequence. Following extension of the immobilized oligonucleotide and generation of an immobilized complement of the target polynucleotide, the immobilized complement includes any primer binding sequence present on the immobilized oligonucleotide. In embodiments, the first endogenous region of the target polynucleotide does not include a universal primer binding sequence. In embodiments, the first endogenous region of the target polynucleotide does not include an adapter. In embodiments, the second region of the target polynucleotide does not include a universal primer binding sequence. In embodiments, the second region of the target polynucleotide does not include an adapter. In embodiments, the second endogenous region of the target polynucleotide does not include a universal primer binding sequence. In embodiments, the second endogenous region of the target polynucleotide does not include an adapter. In embodiments, the first endogenous region of the target polynucleotide does not include a synthetic sequence. In embodiments, the second endogenous region of the target polynucleotide does not include a synthetic sequence.

The target polynucleotide molecules may be treated chemically or enzymatically either prior or subsequent to any fragmentation processes, and prior to or subsequent to the step of enrichment using the probe libraries or probe amplicons. Such fragmentation methods are known in the art and can utilize standard methods (Sambrook and Russell, Molecular Cloning, A Laboratory Manual, third edition). Random fragmentation is designed to produce fragments irrespective of the sequence identity or position of nucleotides including and/or surrounding the break. More particularly, the random fragmentation can be by mechanical means such as nebulization or sonication. The fragments can be about 50 base pairs in length to about 1500 base pairs in length. In embodiments, the target polynucleotides are 50-700 base pairs in length, or 50-400 base pairs in length. In embodiments, the target polynucleotides are about 100-300 base pairs in length. In the methods described herein, the nucleic acid samples may be fragmented prior to hybridization on the array or may be used without fragmentation. The samples may be subjected to an amplification reaction prior to use, for example a whole sample amplification technique such as random primer extension.

In embodiments, the target polynucleotide includes primer binding sequences that are complementary to one or more substrate-bound primers following a) and b) (e.g., binding the first target binding sequence to the target polynucleotide and extending the first primer to generate a first complement of the target polynucleotide template includes the first platform primer binding sequence and a complementary sequence of the target polynucleotide; (b) binding the second target binding sequence to the first complement of the target polynucleotide and extending the second primer to generate a second template includes a first platform primer sequence, a sequence of the target polynucleotide, and the second platform primer sequence). In embodiments, the substrate-bound primers are immobilized to a substrate by a covalent linker. In embodiments, the substrate-bound primers are immobilized to a solid support at the 5′ end, preferably via a covalent attachment. In embodiments, at least some of the substrate-bound primers are phosphorothioated primers. In embodiments, a fraction of the total of the substrate-bound primers are phosphorothioated primers. In embodiments, at least some of the immobilized primers are phosphorothioated primers. In embodiments, a fraction of the total of the immobilized primers are phosphorothioated primers.

In embodiments, the primer is covalently attached to the solid support. In embodiments, the 5′ end of the primer contains a functional group that is tethered to the solid support. Non-limiting examples of covalent attachment include amine-modified polynucleotides reacting with epoxy or isothiocyanate groups on the solid support, succinylated polynucleotides reacting with aminophenyl or aminopropyl functional groups on the solid support, dibenzocycloctyne-modified polynucleotides reacting with azide functional groups on the solid support (or vice versa), trans-cyclooctyne-modified polynucleotides reacting with tetrazine or methyl tetrazine groups on the solid support (or vice versa), disulfide modified polynucleotides reacting with mercapto-functional groups on the solid support, amine-functionalized polynucleotides reacting with carboxylic acid groups on the solid support via 1-ethyl-3-(3-dimethylaminopropyl)-carbodiimide hydrochloride (EDC) chemistry, thiol-modified polynucleotides attaching to a solid support via a disulphide bond or maleimide linkage, alkyne-modified polynucleotides attaching to a solid support via copper-catalyzed click reactions to azide functional groups on the solid support, and acrydite-modified polynucleotides polymerizing with free acrylic acid monomers on the solid support to form polyacrylamide or reacting with thiol groups on the solid support. In embodiments, the primer is attached to the solid support polymer through electrostatic binding. For example, the negatively charged phosphate backbone of the primer may be bound electrostatically to positively charged monomers in the solid support. In embodiments, the primer includes a first bioconjugate reactive group. In embodiments, the primer is attached to a cellular compartment. In embodiments, the cellular component includes a second bioconjugate reactive group. In embodiments, the first bioconjugate reactive group is attached to the second bioconjugate reactive group by covalent or non-covalent bonding. In embodiments, the primer is covalently attached to a cellular component. In embodiments, the 5′ end of the primer contains a functional group that is tethered to the cellular component. In embodiments, the primer is covalently attached to a matrix within the cell. In embodiments, the 5′ end of the primer contains a functional group that is tethered to the matrix within the cell. Non-limiting examples of covalent attachment include amine-modified polynucleotides reacting with epoxy or isothiocyanate groups in the cell or matrix within the cell, succinylated polynucleotides reacting with aminophenyl or aminopropyl functional groups in the cell or matrix within the cell, dibenzocycloctyne-modified polynucleotides reacting with azide functional groups in the cell or matrix within the cell (or vice versa), trans-cyclooctyne-modified polynucleotides reacting with tetrazine or methyl tetrazine groups in the cell or matrix within the cell (or vice versa), disulfide modified polynucleotides reacting with mercapto-functional groups in the cell or matrix within the cell, amine-functionalized polynucleotides reacting with carboxylic acid groups in the cell or matrix within the cell via 1-ethyl-3-(3-dimethylaminopropyl)-carbodiimide hydrochloride (EDC) chemistry, thiol-modified polynucleotides attaching to cell or matrix within the cell via a disulphide bond or maleimide linkage, alkyne-modified polynucleotides attaching to the cell or matrix within the cell via copper-catalyzed click reactions to azide functional groups in the cell or matrix within the cell, and acrydite-modified polynucleotides polymerizing with free acrylic acid monomers in the cell or matrix within the cell to form polyacrylamide or reacting with thiol groups in the cell or matrix within the cell. In embodiments, the primer is attached to the polymer through electrostatic binding. For example, the negatively charged phosphate backbone of the primer may be bound electrostatically to positively charged monomers in the solid support.

In embodiments, the first target binding sequence and/or the second target binding sequence include a melting temperature of about 60 °C, 65 °C, 70 °C, or about 75 °C. In embodiments, the first target binding sequence and/or the second target binding sequence include a melting temperature of about 60 °C, 65 °C, 70 °C, or about 75 °C. In embodiments, the first target binding sequence and/or the second target binding sequence include a substantially similar melting temperature. In embodiments, the first target binding sequence and/or the second target binding sequence include a melting temperature of about 65 °C to about 75 °C.

In embodiments, the first target binding sequence is about 20, about 30, about 35, about 40, about 45, about 50, or about 55 nucleotides in length. In embodiments, the first binding sequence is about 30, about 35, about 40, about 45, about 50, or about 55 nucleotides. In embodiments, the first target binding sequence is about 35 nucleotides in length. In embodiments, the first target binding sequence is about 36 nucleotides in length. In embodiments, the first target binding sequence is about 37 nucleotides in length. In embodiments, the first target binding sequence is about 38 nucleotides in length. In embodiments, the first target binding sequence is about 39 nucleotides in length. In embodiments, the first target binding sequence is about 40 nucleotides in length. In embodiments, the first target binding sequence is about 41 nucleotides in length. In embodiments, the first target binding sequence is about 42 nucleotides in length. In embodiments, the first target binding sequence is about 43 nucleotides in length. In embodiments, the first target binding sequence is about 44 nucleotides in length. In embodiments, the first target binding sequence is about 45 nucleotides in length.

In embodiments, the first target binding sequence includes a sequence that is at least 80%, at least 85%, at least 90%, or at least 95% identical to a continuous 30 nucleotide sequence within SEQ ID NO: 7; at least 80%, at least 85%, at least 90%, or at least 95% identical to a continuous 30 nucleotide sequence within SEQ ID NO:9; at least 80%, at least 85%, at least 90%, or at least 95% identical to a continuous 30 nucleotide sequence within SEQ ID NO:11; at least 80%, at least 85%, at least 90%, or at least 95% identical to a continuous 30 nucleotide sequence within SEQ ID NO:13; at least 80%, at least 85%, at least 90%, or at least 95% identical to a continuous 30 nucleotide sequence within SEQ ID NO:15; at least 80%, at least 85%, at least 90%, or at least 95% identical to a continuous 30 nucleotide sequence within SEQ ID NO: 17; at least 80%, at least 85%, at least 90%, or at least 95% identical to a continuous 30 nucleotide sequence within SEQ ID NO:19; at least 80%, at least 85%, at least 90%, or at least 95% identical to a continuous 30 nucleotide sequence within SEQ ID NO:21; at least 80%, at least 85%, at least 90%, or at least 95% identical to a continuous 30 nucleotide sequence within SEQ ID NO:23; at least 80%, at least 85%, at least 90%, or at least 95% identical to a continuous 30 nucleotide sequence within SEQ ID NO:25; at least 80%, at least 85%, at least 90%, or at least 95% identical to a continuous 30 nucleotide sequence within SEQ ID NO:27; at least 80%, at least 85%, at least 90%, or at least 95% identical to a continuous 30 nucleotide sequence within SEQ ID NO:29; at least 80%, at least 85%, at least 90%, or at least 95% identical to a continuous 30 nucleotide sequence within SEQ ID NO:31; at least 80%, at least 85%, at least 90%, or at least 95% identical to a continuous 30 nucleotide sequence within SEQ ID NO:33; at least 80%, at least 85%, at least 90%, or at least 95% identical to a continuous 30 nucleotide sequence within SEQ ID NO:35; at least 80%, at least 85%, at least 90%, or at least 95% identical to a continuous 30 nucleotide sequence within SEQ ID NO:37; at least 80%, at least 85%, at least 90%, or at least 95% identical to a continuous 30 nucleotide sequence within SEQ ID NO: 39; at least 80%, at least 85%, at least 90%, or at least 95% identical to a continuous 30 nucleotide sequence within SEQ ID NO:41; at least 80%, at least 85%, at least 90%, or at least 95% identical to a continuous 30 nucleotide sequence within SEQ ID NO:43; at least 80%, at least 85%, at least 90%, or at least 95% identical to a continuous 30 nucleotide sequence within SEQ ID NO:45; at least 80%, at least 85%, at least 90%, or at least 95% identical to a continuous 30 nucleotide sequence within SEQ ID NO:47; at least 80%, at least 85%, at least 90%, or at least 95% identical to a continuous 30 nucleotide sequence within SEQ ID NO:49; at least 80%, at least 85%, at least 90%, or at least 95% identical to a continuous 30 nucleotide sequence within SEQ ID NO:51; at least 80%, at least 85%, at least 90%, or at least 95% identical to a continuous 30 nucleotide sequence within SEQ ID NO:53; at least 80%, at least 85%, at least 90%, or at least 95% identical to a continuous 30 nucleotide sequence within SEQ ID NO:55; at least 80%, at least 85%, at least 90%, or at least 95% identical to a continuous 30 nucleotide sequence within SEQ ID NO:57; or at least 80%, at least 85%, at least 90%, or at least 95% identical to a continuous 30 nucleotide sequence within SEQ ID NO:59.

In embodiments, the 3′ end of the first target binding sequence is complementary to the first endogenous region of the target polynucleotide, and the 3′ end of the target binding sequence is complementary to the complement of the second region of the target polynucleotide. In embodiments, the 3′ end of the first target binding sequence is complementary to the first endogenous region of the target polynucleotide. In embodiments, the 3′ end of the target binding sequence is complementary to the complement of the second region of the target polynucleotide.

In embodiments, the second target binding sequence is identical to a second endogenous region of the target polynucleotide. In embodiments, the second target binding sequence is 80%, 90%, 95%, 99%, or 100% identical to a second region of the target polynucleotide. In embodiments, the second target binding sequence is 80% identical to a second region of the target polynucleotide. In embodiments, the second target binding sequence is 90% identical to a second region of the target polynucleotide. In embodiments, the second target binding sequence is 95% identical to a second region of the target polynucleotide. In embodiments, the second target binding sequence is 99% identical to a second region of the target polynucleotide. In embodiments, the second target binding sequence is 95% identical to a second region of the target polynucleotide. In embodiments, the second target binding sequence is 100% identical to a second region of the target polynucleotide.

In embodiments, the second target binding sequence is about 20, about 30, about 35, about 40, about 45, about 50, or about 55 nucleotides in length. In embodiments, the second binding sequence is about 30, about 35, about 40, about 45, about 50, or about 55 nucleotides. In embodiments, the second target binding sequence is about 35 nucleotides in length. In embodiments, the second target binding sequence is about 36 nucleotides in length. In embodiments, the second target binding sequence is about 37 nucleotides in length. In embodiments, the second target binding sequence is about 38 nucleotides in length. In embodiments, the second target binding sequence is about 39 nucleotides in length. In embodiments, the second target binding sequence is about 40 nucleotides in length. In embodiments, the second target binding sequence is about 41 nucleotides in length. In embodiments, the second target binding sequence is about 42 nucleotides in length. In embodiments, the second target binding sequence is about 43 nucleotides in length. In embodiments, the second target binding sequence is about 44 nucleotides in length. In embodiments, the second target binding sequence is about 45 nucleotides in length.

In embodiments, the second target binding sequence includes a sequence that is at least 80%, at least 85%, at least 90%, or at least 95% identical to a continuous 30 nucleotide sequence within SEQ ID NO:8; at least 80%, at least 85%, at least 90%, or at least 95% identical to a continuous 30 nucleotide sequence within SEQ ID NO:10; at least 80%, at least 85%, at least 90%, or at least 95% identical to a continuous 30 nucleotide sequence within SEQ ID NO:12; at least 80%, at least 85%, at least 90%, or at least 95% identical to a continuous 30 nucleotide sequence within SEQ ID NO:14; at least 80%, at least 85%, at least 90%, or at least 95% identical to a continuous 30 nucleotide sequence within SEQ ID NO: 16; at least 80%, at least 85%, at least 90%, or at least 95% identical to a continuous 30 nucleotide sequence within SEQ ID NO: 18; at least 80%, at least 85%, at least 90%, or at least 95% identical to a continuous 30 nucleotide sequence within SEQ ID NO:20; at least 80%, at least 85%, at least 90%, or at least 95% identical to a continuous 30 nucleotide sequence within SEQ ID NO:22; at least 80%, at least 85%, at least 90%, or at least 95% identical to a continuous 30 nucleotide sequence within SEQ ID NO:24; at least 80%, at least 85%, at least 90%, or at least 95% identical to a continuous 30 nucleotide sequence within SEQ ID NO:26; at least 80%, at least 85%, at least 90%, or at least 95% identical to a continuous 30 nucleotide sequence within SEQ ID NO:28; at least 80%, at least 85%, at least 90%, or at least 95% identical to a continuous 30 nucleotide sequence within SEQ ID NO:30; at least 80%, at least 85%, at least 90%, or at least 95% identical to a continuous 30 nucleotide sequence within SEQ ID NO:32; at least 80%, at least 85%, at least 90%, or at least 95% identical to a continuous 30 nucleotide sequence within SEQ ID NO:34; at least 80%, at least 85%, at least 90%, or at least 95% identical to a continuous 30 nucleotide sequence within SEQ ID NO:36; at least 80%, at least 85%, at least 90%, or at least 95% identical to a continuous 30 nucleotide sequence within SEQ ID NO:38; at least 80%, at least 85%, at least 90%, or at least 95% identical to a continuous 30 nucleotide sequence within SEQ ID NO: 40; at least 80%, at least 85%, at least 90%, or at least 95% identical to a continuous 30 nucleotide sequence within SEQ ID NO:42; at least 80%, at least 85%, at least 90%, or at least 95% identical to a continuous 30 nucleotide sequence within SEQ ID NO:44; at least 80%, at least 85%, at least 90%, or at least 95% identical to a continuous 30 nucleotide sequence within SEQ ID NO:46; at least 80%, at least 85%, at least 90%, or at least 95% identical to a continuous 30 nucleotide sequence within SEQ ID NO:48; at least 80%, at least 85%, at least 90%, or at least 95% identical to a continuous 30 nucleotide sequence within SEQ ID NO:50; at least 80%, at least 85%, at least 90%, or at least 95% identical to a continuous 30 nucleotide sequence within SEQ ID NO:52; at least 80%, at least 85%, at least 90%, or at least 95% identical to a continuous 30 nucleotide sequence within SEQ ID NO:54; at least 80%, at least 85%, at least 90%, or at least 95% identical to a continuous 30 nucleotide sequence within SEQ ID NO:56; at least 80%, at least 85%, at least 90%, or at least 95% identical to a continuous 30 nucleotide sequence within SEQ ID NO:58; or at least 80%, at least 85%, at least 90%, or at least 95% identical to a continuous 30 nucleotide sequence within SEQ ID NO:60.

In embodiments, the third primer is about 10 nucleotides to about 35 nucleotides. In embodiments, the third primer is about 15 nucleotides to about 30 nucleotides. In embodiments, the third primer is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, or about 50 nucleotides in length.

In embodiments, the fourth primer is about 10 nucleotides to about 35 nucleotides. In embodiments, the fourth primer is about 15 nucleotides to about 30 nucleotides. In embodiments, the third primer is about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 36 nucleotides, about 37 nucleotides, about 38 nucleotides, about 39 nucleotides, about 40 nucleotides, about 41 nucleotides, about 42 nucleotides, about 43 nucleotides, about 44 nucleotides, about 45 nucleotides, about 46 nucleotides, about 47 nucleotides, about 48 nucleotides, about 49 nucleotides, or about 50 nucleotides in length.

In embodiments, the primer binding sequence of the third primer includes a different primer binding sequence than the primer binding sequence of fourth primer. In embodiments, the primer binding sequence is common (e.g., identical or universal) to the immobilized oligonucleotides of the array such that all of the immobilized nucleotides may initiate amplification by annealing and extending the same primer.

In embodiments, the polymerase is a strand-displacing or non-strand displacing polymerase. In embodiments, the polymerase is a strand-displacing polymerase. In embodiments, the strand-displacing polymerase is Bst large fragment (Bst LF) polymerase, Bst 3.0 polymerase, Bst2.0 polymerase, Bsu polymerase, SD polymerase, Vent exo-polymerase, Phi29 polymerase, or a mutant thereof. In embodiments, the polymerase is Bst DNA Polymerase, Vent (exo-) DNA Polymerase, Pfu DNA polymerase, Taq polymerase, Phusion High-Fidelity DNA Polymerase, Q5 High-Fidelity DNA Polymerase, or mutant of any one of the foregoing. In embodiments, the polymerase is Bst DNA Polymerase, Vent (exo-) DNA Polymerase, Phusion High-Fidelity DNA Polymerase, or Q5 High-Fidelity DNA Polymerase. In embodiments, the polymerase is a Pyrococcus polymerase (e.g., a polymerase described in WO 2018/148723 or WO 2020/056044, each of which are incorporated herein by reference for all purposes). In embodiments, the polymerase is a Bst DNA polymerase (e.g., exonuclease minus Bst), phi29 DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In embodiments, the polymerase is a phi29 DNA polymerase wild type phi29 DNA polymerase (e.g., MagniPhi from Expedeon), or variant EquiPhi29 DNA polymerase (e.g., from Thermo Fisher Scientific), or chimeric QualiPhi DNA polymerase (e.g., from 4basebio). In embodiments, the polymerase is a non-strand displacing polymerase. In embodiments, the non-strand displacing polymerase is T4 DNA polymerase. In embodiments, the non-strand displacing polymerase is T7 DNA polymerase.

In embodiments, the method further includes sequencing the immobilized polynucleotide. For example, the method includes sequencing the target polynucleotide sequence, or the complement thereof. In embodiments, sequencing includes sequencing-by-synthesis, sequencing-by-binding, sequencing by ligation, or pyrosequencing, and generates a sequencing read. In embodiments, the method further includes sequencing-by-synthesis and generates a sequencing read. In embodiments, the method includes sequencing by ligation and generates a sequencing read. In embodiments, the method includes pyrosequencing and generates a sequencing read. In embodiments, the method includes sequencing-by-binding and generates a sequencing read.

In embodiments, sequencing includes contacting the immobilized polynucleotide with a sequencing primer, extending the sequencing primer with a polymerase to incorporate a detectable label (e.g., a nucleotide including a detectable label) that indicates the identity of a nucleotide in the target polynucleotide, detecting the detectable label, and repeating the extending and detecting steps, wherein the sequencing primer is complementary to a primer binding sequence of the third primer or fourth primer.

In embodiments, the detectable agent has a maximum excitation wavelength between 350-400 nm, between 400-450 nm, between 450-500 nm, between 500-550 nm, between 550-600 nm, between 600-650 nm, between 650-700 nm, 700-750 nm, or between 750-800 nm. In embodiments, the detectable agent has a maximum excitation wavelength of about 325 nm, 343 nm, 350 nm, 353 nm, 359 nm, 360 nm, 395 nm, 400 nm, 401 nm, 402 nm, 403 nm, 425 nm, 434 nm, 440 nm, 466 nm, 480 nm, 485 nm, 489 nm, 490 nm, 492 nm, 493 nm, 494 nm, 495 nm, 496 nm, 498 nm, 499 nm, 500 nm, 502 nm, 503 nm, 505 nm, 517 nm, 518 nm, 520 nm, 525 nm, 528 nm, 530 nm, 531 nm, 535 nm, 542 nm, 544 nm, 547 nm, 550 nm, 553 nm, 554 nm, 558 nm, 560 nm, 561 nm, 562 nm, 565 nm, 567 nm, 570 nm, 572 nm, 579 nm, 581 nm, 589 nm, 590 nm, 591 nm, 593 nm, 596 nm, 610 nm, 631 nm, 632 nm, 638 nm, 650 nm, 652 nm, 654 nm, 663 nm, 675 nm, 680 nm, 692 nm, 696 nm, 743 nm, 752 nm, 777 nm, or 782 nm.

In embodiments, the detectable agent has a maximum emission wavelength between 400-450 nm, between 450-500 nm, between 500-550 nm, between 550-600 nm, between 600-650 nm, between 650-700 nm, between 700-750 nm, between 750-800 nm, or between 800-850 nm. In embodiments, the detectable agent has a maximum emission of about 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm.

In embodiments, the solid support includes a plurality of primers attached to the solid support, wherein each of the primers includes a sequence complementary to the first platform primer sequence. In embodiments, the solid support includes a plurality of primers attached to the solid support, wherein each of the primers includes a sequence complementary to the second platform primer sequence.

In embodiments, the solid support further includes a fourth primer immobilized to the solid support, wherein said fourth primer comprises a sequence complementary to the first platform primer sequence. In embodiments, the solid support further includes a fourth primer immobilized to the solid support, wherein said fourth primer comprises a sequence complementary to the second platform primer sequence.

In embodiments, the platform primer sequence includes a platform primer used for cluster generation and sequencing on a G4™ sequencing platform from Singular Genomics® include SP2′: 5′-GATCGGAAGAGCACACGTCTGAACTCCAGTC (SEQ ID NO:1), S2′: 5′-AGTGGTTGGTGAGGGTCATCTCGCTGGAG (SEQ ID NO:2), S1: 5′-ACAAAGGCAGCCACGCACTCCTTCCCTGT (SEQ ID NO:3), and SP1: ACACTCTTTCCCTACACGACGCTCTTCCGATCT (SEQ ID NO:4), or a complement thereof. In embodiments, the platform primer sequence includes a platform primer used on the surface of commercial flow cells for sequencing on various Illumina platforms, as described in U.S. Patent Publication No. 2011/0059865 A1, including P5 and P7 primers (e.g., P5: 5′-AATGATACGGCGACCACCG (SEQ ID NO: 5), or the complement thereof, and P7: 5′-CAAGCAGAAGACGGCATACGA (SEQ ID NO: 6), or the complement thereof). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length. The primer binding site may be selected such that the primer (e.g., sequencing primer) has the following properties, for example having a length of about 20-30 nucleotides; approximately 50% GC content, and a Tm of about 55 °C to about 65 °C.

In embodiments, the solid support is a flow cell. In embodiments, the flow cell includes a flow cell assembly including a frame configured to retain the flow cell assembly. Suitable flow cell frames and handles are described in U.S. Pat. No. 11,747,262. The frame can be configured to retain the flow cell such that a maximal surface area of the flow cell can be available to be exposed to an optical lens (e.g., the optical lens of a nucleic acid sequencing device). The optical lens (e.g., the optical lens of the sequencing device) can be configured to detect excitation, emission, or other signals present on the flow cell. The frame can be configured to retain the flow cell such that a maximal surface area of the flow cell can be available to be in contact with the receiver of a nucleic acid sequencer. The retaining of the flow cell further can include constraining a first, a second, a third, a fourth, a fifth, and a sixth degree of freedom of the flow cell. The frame can be an injection molded frame. The handle can be a raised handle. The frame can be further configured to provide a gap between a work surface and the flow cell. The frame further can include at least one ferromagnetic pin. The at least one biasing feature can be a spring finger. The at least one biasing feature can be a tab. The flow cell can further include a microchip. The microchip can be an electronically erasable programmable read only memory (EEPROM) chip.

In embodiments, the flow cell includes a channel bored into the flow cell. In embodiments, the flow cell includes a channel bored into the flow cell. In embodiments, the flow cell includes 2 channels bored into the flow cell. In embodiments, the flow cell includes 3 channels bored into the flow cell. In embodiments, the flow cell includes 4 channels bored into the flow cell. In embodiments, the flow cell includes a plurality of channels bored into the flow cell.

In embodiments, the flow cell includes a plurality of nanowells. In embodiments, each nanowell is about 0.1 μm to about 2.0 μm in depth, and wherein the nanowells are about 0.1 μm to about 2.0 μm in diameter. In embodiments, each nanowell is about 0.1 μm, 0.2 μm, 0.3 μm, 0.4μ, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, or 2.0 μm in depth. In embodiments, each nanowell is about 0.1μ, 0.2 μm, 0.3 μm, 0.4 μm, 0.5μ, 0.6 μm, 0.7 μm, 0.8 μm, 0.9 μm, 1.0 μm, 1.1μm, 1.2 μm, 1.3 μm, 1.4 μm, 1.5 μm, 1.6 μm, 1.7 μm, 1.8 μm, 1.9 μm, or 2.0 μm in diameter.

In an aspect is a method of amplifying a polynucleotide, the method including: contacting a solid support of (e.g., a flow cell as described herein) with a plurality of polynucleotides, wherein each of the plurality of polynucleotides include a first platform primer binding sequence, a target sequence, and a second platform primer sequence and do not include a common primer binding sequence, wherein the solid support includes a first plurality of immobilized oligonucleotides each including a first platform primer sequence and a second plurality of immobilized oligonucleotides each including a second platform primer sequence; hybridizing the first platform primer binding sequence of a polynucleotide of the plurality of polynucleotides to a first immobilized oligonucleotide from the first plurality, and extending with a polymerase the first immobilized oligonucleotide to generate a first extension product; hybridizing the first extension product to a second immobilized oligonucleotide from the second plurality, and extending with a polymerase the second immobilized oligonucleotide to generate a second extension product; and hybridizing the second extension product to a third immobilized oligonucleotide from the first plurality, and extending with a polymerase the third immobilized oligonucleotide to generate a third extension product. In embodiments, the method includes sequencing the first extension product, the second extension product, and/or the third extension product. In embodiments, the method does not include substantial library preparation (e.g., DNA-preprocessing) methods.

Hybridizing a target polynucleotide or sequence thereof (e.g., a platform binding sequence) to a polynucleotide primer (e.g., an immobilized oligonucleotide) is an inherently stochastic event. For stochastic events occurring over a period of time (e.g., a seeding-amplification cycle) it may be convenient to use the Poisson approximation to better understand the probability of an event occurring during that time. For example, if one knows the average rate of a hybridization event, represented as λ^{seed}, (i.e., how often a target polynucleotide hybridizes to a polynucleotide primer) occurring during a seeding-amplification cycle, it is possible to calculate the probability that an amplification site will contain an amplicon (e.g., a monoclonal amplicon) following a seeding-amplification cycle. Two variables affecting A seed include the concentration of the target polynucleotide and the amount of time the target polynucleotide is exposed to the polynucleotide primer, t_{seed}, during a seeding-amplification cycle. Generally, increasing the concentration of the target polynucleotide or increasing t_{seed} increases λ^{seed}. Conventional methods typically overseed the array, that is, the methods often employed ensure the concentration of the target polynucleotides are in abundance relative to the available amplification sites, to maximize the opportunity for a target polynucleotide to hybridize to the primer in the amplification site. Embodiments described herein suggest underseeding, that is limiting the concentration or limiting the contacting time between target polynucleotides and the primers (i.e., immobilized oligonucleotides), and repeating the cycle to promote capture efficiency.

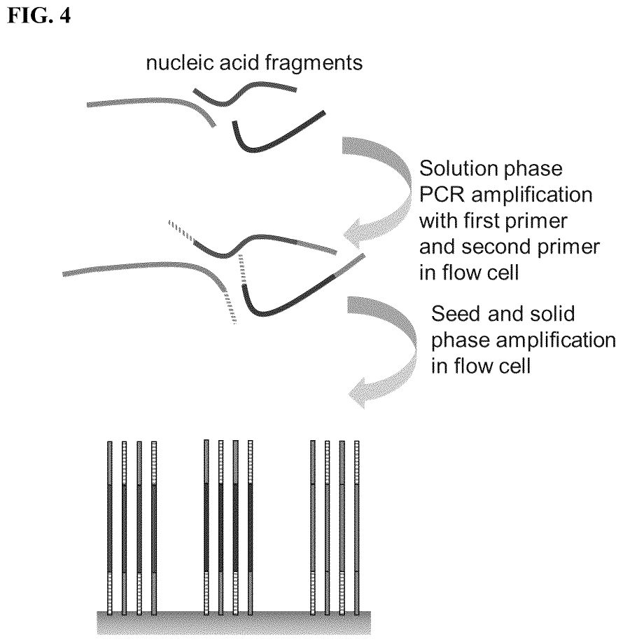

In an aspect is provided a method of generating immobilized complements of a plurality of target polynucleotides of a cell including: a) hybridizing a plurality of target polynucleotides to the first plurality of immobilized oligonucleotides as described herein; and b) extending the immobilized oligonucleotides with a polymerase to generate a plurality of immobilized complements of the plurality of target polynucleotides. See for example FIG. 4.

In an aspect is provided a method of generating immobilized complements of a plurality of target polynucleotides of a cell, wherein each of the plurality of target polynucleotides include a first platform primer binding sequence, a target sequence, and a second platform primer sequence and do not include a common primer binding sequence, wherein the solid support comprises a first plurality of immobilized oligonucleotides each comprising a first platform primer sequence and a second plurality of immobilized oligonucleotides each comprising a second platform primer sequence. In embodiments, the method includes hybridizing the first platform primer binding sequence of a polynucleotide of the plurality of polynucleotides to a first immobilized oligonucleotide from the first plurality, and extending with a polymerase the first immobilized oligonucleotide to generate a first extension product; hybridizing the first extension product to a second immobilized oligonucleotide from the second plurality, and extending with a polymerase the second immobilized oligonucleotide to generate a second extension product; and hybridizing the second extension product to a third immobilized oligonucleotide from the first plurality, and extending with a polymerase the third immobilized polynucleotide.

In embodiments, the target polynucleotide may be derived from any in vivo or in vitro source, including from one or multiple cells, tissues, organs, or organisms, whether living or dead, or from any biological or environmental source (e.g., water, air, soil). For example, in some embodiments, the target polynucleotide includes eukaryotic and/or prokaryotic dsDNA that originates or that are derived from humans, animals, plants, fungi, (e.g., molds or yeasts), bacteria, viruses, viroids, mycoplasma, or other microorganisms. In embodiments, the target polynucleotide includes genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In embodiments, the target polynucleotide is from an in vitro source. For example, in some embodiments, the target polynucleotide includes dsDNA that is prepared in vitro from single-stranded DNA (ssDNA) or from single-stranded or double-stranded RNA (e.g., using methods that are well-known in the art, such as primer extension using a suitable DNA-dependent and/or RNA-dependent DNA polymerase (reverse transcriptase). In some embodiments, the target polynucleotide includes dsDNA that is prepared from all or a portion of one or more double-stranded or single-stranded DNA or RNA molecules using any methods known in the art, including methods for: DNA or RNA amplification (e.g., PCR or reverse-transcriptase-PCR (RT-PCR), transcription-mediated amplification methods, with amplification of all or a portion of one or more nucleic acid molecules); molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray.

In embodiments, prior to step a), the target polynucleotide does not include a primer binding sequence (or complementary sequence thereof) on a 5′ or 3′ end. In embodiments, prior to step a), the target polynucleotide does not include end-polishing (e.g., generating blunt-ends). In embodiments, prior to step a), target polynucleotide does not include contacting the targeting polynucleotides with a ligase. In embodiments, prior to step a), the target polynucleotide does not include contacting the targeting polynucleotides with a transposase. In embodiments, the method includes adding a common primer binding sequence to the target polynucleotide in the reaction vessel, and following the inclusion of a common primer binding sequence to each target polynucleotide, within the same reaction vessel, binding the common primer binding sequence of the target polynucleotide to an immobilized oligonucleotide. In embodiments, the method includes extending the immobilized oligonucleotide to generate an immobilized complement of the target polynucleotide. In embodiments, the reaction vessel is a flow cell. In embodiments, the reaction vessel is a channel of a flow cell (e.g., a 4-channel flow cell).

It will be appreciated that any of the amplification methodologies described herein or known in the art can be utilized with universal or target-specific primers to amplify the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification, RCA with exponential strand displacement amplification. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the flow cell for enabling or accelerating amplification.

In embodiments, amplifying includes contacting the solid support (e.g., the flow cell and/or a plurality of particles) with one or more reagents for amplifying the target polynucleotide. Examples of reagents include but are not limited to polymerase, buffer, and nucleotides (e.g., an amplification reaction mixture). In embodiments the term “amplifying” refers to a method that includes a polymerase chain reaction (PCR) including one or more cycles of annealing, extension, and denaturation. Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101 (43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the polymerase is a strand-displacing polymerase.

In embodiments, amplifying includes performing 1 round, 2 rounds, 3 rounds, 4 rounds, 5 rounds, 6 rounds, 7 rounds, 8 rounds, 9 rounds, 10 rounds, 11 rounds, 12 rounds, 13 rounds, 14 rounds, 15 rounds, 16 rounds, 17 rounds, 18 rounds, 19 rounds, 20 rounds, 21 rounds, 22 rounds, 23 rounds, 24 rounds, 25 rounds, 26 rounds, 27 rounds, 28 rounds, 29 rounds, or 30 rounds of PCR. In embodiments, amplifying includes performing about 10 rounds of PCR. In embodiments, amplifying includes performing about 15 rounds of PCR. In embodiments, amplifying includes performing about 20 rounds of PCR. In embodiments, amplifying includes performing about 25 rounds of PCR. In embodiments, the method does not include any purification step following the amplification step.

In embodiments, the method includes amplifying the circular polynucleotide by extending an amplification primer with a strand-displacing polymerase, wherein the primer extension generates an extension product including multiple complements of the circular polynucleotide. In embodiments, the method of amplifying includes an isothermal amplification method. In embodiments, the method of amplifying includes rolling circle amplification (RCA) or rolling circle transcription (RCT). In embodiments, the method of amplifying is rolling circle amplification (RCA). In embodiments, amplifying includes exponential rolling circle amplification (eRCA). Exponential RCA is similar to the linear process except that it uses a second primer (e.g., one or more immobilized oligonucleotide(s)) having a sequence that is identical to at least a portion of the circular template (Lizardi et al. Nat. Genet. 19:225 (1998)). This two-primer system achieves isothermal, exponential amplification. In embodiments, amplifying the circular oligonucleotide includes incubation with a strand-displacing polymerase. In embodiments, amplifying includes incubation with a strand-displacing polymerase for about 10 seconds to about 30 minutes. In embodiments, amplifying includes incubation with a strand-displacing polymerase at a temperature of about 20 °C to about 50 °C. In embodiments, incubation with the strand-displacing polymerase is at a temperature of about 35 °C to 42 °C. In embodiments, the strand-displacing polymerase is phi29 polymerase, SD polymerase, Bst large fragment polymerase, phi29 mutant polymerase, or a thermostable phi29 mutant polymerase. In embodiments, the extension product includes three or more copies of the target nucleic acid. In embodiments, the extension product includes at least three or more copies of the target nucleic acid. In embodiments, the extension product includes at least five or more copies of the target nucleic acid. In embodiments, the extension product includes at 5 to 10 copies of the target nucleic acid. In embodiments, the extension product includes 10 to 20 copies of the target nucleic acid. In embodiments, the extension product includes 20 to 50 copies of the target nucleic acid.

In embodiments, the method includes forming the circular polynucleotide by hybridizing a first end and a second end of the target polynucleotide to the immobilized primer (e.g., the third primer) and ligating the first end and second end together to form the circular polynucleotide. In embodiments, hybridizing a first end and a second end of a single-stranded polynucleotide described herein to a nucleic acid molecule described herein includes incubation in a buffer at about 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, 50 °C, 51 °C, 52 °C, 53 °C, 54 °C, 55 °C, 56 °C, 57 °C, 58 °C, 59 °C, 60 °C, 61 °C, 62 °C, 63 °C, 64 °C, or 65 °C. In embodiments, hybridizing a first end and a second end of a single-stranded polynucleotide described herein to a nucleic acid molecule described herein includes incubation in a buffer at 40 °C to 50 °C. In embodiments, hybridizing a first end and a second end of a single-stranded polynucleotide described herein to a nucleic acid molecule described herein includes incubation in a buffer at 50 °C to 60 °C. In embodiments, ligating includes incubation in a buffer at about 20 °C, 21 °C, 22 °C, 23 °C, 24 °C, 25 °C, 26 °C, 27 °C, 28 °C, 29 °C, 30 °C, 31 °C, 32 °C, 33 °C, 34 °C, 35 °C, 36 °C, 37 °C, 38 °C, 39 °C, 40 °C, 41 °C, 42 °C, 43 °C, 44 °C, 45 °C, 46 °C, 47 °C, 48 °C, 49 °C, 50 °C, 51 °C, 52 °C, 53 °C, 54 °C, 55 °C, 56 °C, 57 °C, 58 °C, 59 °C, 60 °C, 61 °C, 62 °C, 63 °C, 64 °C, or 65 °C. In embodiments, ligating includes incubation in a buffer 30 °C to 40 °C. In embodiments, ligating includes incubation in a buffer 40 °C to 50 °C. In embodiments, the method includes forming the circular polynucleotide by hybridizing a first end and a second end of the target polynucleotide to the immobilized primer, extending the second end along the immobilized primer, and ligating the first end and the extended second end together to form the circular polynucleotide. For example, the first end includes a first hybridization sequence and the second end includes a second hybridization sequence, wherein each hybridization sequence is complementary to a respective sequence of the immobilized nucleic acid molecule which flanks one or more nucleotides (e.g., a gap). In embodiments, the gap may include a barcode sequence. In embodiments, the circular polynucleotide includes a barcode sequence. In embodiments, the circular polynucleotide does not include a barcode sequence. In embodiments, the barcode (i.e., the barcode sequence) is at least 5, 6, 7, 8, 9, 10, 11, 12, 13, 14, or 15 nucleotides in length. In embodiments, the barcode is 5, 6, 7, 8, 9, 10, 11, 12, 13, 14, or 15 nucleotides in length. In embodiments, the barcode is 10 to 15 nucleotides in length. In embodiments, the barcode is at least about 1, 2, 3, 4, 5, 6, 7, 8, 9, 10, 15, 20, 25, 30, 35, 40, 45, 50 or more nucleotides in length. In embodiments, the barcode can be at most about 300, 200, 100, 90, 80, 70, 60, 50, 40, 30, 20, 15, 12, 10, 9, 8, 7, 6, 5, 4 or fewer or more nucleotides in length. In embodiments, the barcode includes between about 5 to about 8, about 5 to about 10, about 5 to about 15, about 5 to about 20, about 10 to about 150 nucleotides. In embodiments, the barcode includes between 5 to 8, 5 to 10, 5 to 15, 5 to 20, 10 to 150 nucleotides. In embodiments, the barcode is 10 nucleotides. In embodiments, the barcode may include a unique sequence (e.g., a barcode sequence) that gives the barcode its identifying functionality. The unique sequence may be random or non-random. In embodiments, the barcode is known (i.e., the nucleic sequence is known before sequencing) and is sorted into a basis-set according to their Hamming distance. Oligonucleotide barcodes (e.g., barcode sequences included in an oligonucleotide) can be associated with a target of interest by knowing, a priori, the target of interest, such as a gene or protein. In embodiments, the barcodes further include one or more sequences capable of specifically binding a gene or nucleic acid sequence of interest.

In embodiments, the primers may be used to aid in tethering the extension product to a confined area and may not be extended. In embodiments, the immobilized oligonucleotides include blocking groups at their 3′ ends that prevent polymerase extension. A blocking moiety prevents formation of a covalent bond between the 3′ hydroxyl moiety of the nucleotide and the 5′ phosphate of another nucleotide. In embodiments, the method includes extending the one or more immobilized oligonucleotides hybridized to an extension product with a polymerase. For example, the one or more immobilized oligonucleotides may be used to aid in tethering the extension product to a localized area and may extended in an exponential RCA amplification reaction.

In embodiments, the method includes circularizing the single-stranded polynucleotide described herein to form a circular polynucleotide described herein. In embodiments, the circularizing includes intramolecular joining of the 5′ and 3′ ends of a linear nucleic acid molecule (e.g., the single-stranded target polynucleotide following the additional of the platform binding sequences described herein). In embodiments, the circularizing includes a ligation reaction. In embodiments, the two ends of the linear nucleic acid molecule are ligated directly together. In embodiments, the two ends of the linear nucleic acid molecule are ligated together with the aid of a bridging oligonucleotide (sometimes referred to as a splint oligonucleotide) that is complementary with the two ends of the linear nucleic acid molecule. Methods for forming circular DNA templates are known in the art, for example, linear polynucleotides are circularized in a non-template driven reaction with circularizing ligase, such as CircLigase™, CircLigase™ II, Taq DNA Ligase, HiFi Taq DNA Ligase, T4 DNA ligase, or Ampligase® DNA Ligase. In some embodiments, circularization is facilitated by denaturing double-stranded linear nucleic acids prior to circularization. Residual linear DNA molecules may be optionally digested. In some embodiments, circularization is facilitated by chemical ligation (e.g., click chemistry, e.g., a copper-catalyzed reaction of an alkyne (e.g., a 3′ alkyne) and an azide (e.g., a 5′ azide)). In embodiments, prior to circularization, the linear DNA fragments are A-tailed (e.g., A-tailed using Taq DNA polymerase). In embodiments, circularization of the linear nucleic acid molecule is performed with CircLigase™ enzyme.

In embodiments, the method further includes contacting the immobilized target polynucleotides with a sequencing primer and sequencing the immobilized target polynucleotide, and amplification products thereof. In embodiments, sequencing includes generating one or more sequencing reads. Generating sequencing reads may be obtained via, for example, a sequencing-by-synthesis, sequencing-by-binding, or sequencing-by-ligation method.

In embodiments, the method further includes contacting the immobilized target polynucleotides with a sequencing primer, extending the sequencing primer to incorporate a detectable label that indicates the identity of a nucleotide in the target polynucleotides, detecting the detectable label, and repeating the extending and detecting steps, thereby sequencing the target polynucleotides. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing by ligation, or pyrosequencing, and generates a sequencing read.

In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, generating a first sequencing read or a second sequencing read includes sequencing-by-binding (see, e.g., U.S. Pat. Pubs. US2017/0022553, US2019/0048404, and US2021/0373000, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Flow cells provide a convenient format for housing an array of clusters produced by the methods described herein, in particular when subjected to an SBS or other detection technique that involves repeated delivery of reagents in cycles. For example, to initiate a first SBS cycle, one or more labeled nucleotides and a DNA polymerase in a buffer, can be flowed into/through a flow cell that houses an array of clusters. The clusters of an array where primer extension causes a labeled nucleotide to be incorporated can then be detected. Optionally, the nucleotides can further include a reversible termination moiety that temporarily halts further primer extension once a nucleotide has been added to a primer. For example, a nucleotide analog having a reversible terminator moiety can be added to a primer such that subsequent extension cannot occur until a deblocking agent (e.g., a reducing agent) is delivered to remove the moiety. Thus, for embodiments that use reversible termination, a deblocking reagent (e.g., a reducing agent) can be delivered to the flow cell (before, during, or after detection occurs). Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the method includes sequencing the first and/or the second strand of a amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242 (1), 84-9 (1996); Ronaghi, Genome Res. 11 (1), 3-11 (2001); Ronaghi et al. Science 281 (5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135 (3), 303-7 (1988); Drmanac et al., Nature Biotechnology 16, 54-58 (1998); Fodor et al., Science 251 (4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072, 7,541,444 and 7,057,026. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

In embodiments, the method includes determining the nucleic acid sequence of the target polynucleotide. In embodiments, the molecule further includes quantifying the target nucleic acid molecule or amplicons. Methods for quantifying a target polynucleotide or amplicon are well known to one of skilled in the art. For example, during amplification of the target nucleic acid, quantitative techniques such as real-time polymerase chain reaction (RT-PCR) can be used to quantify the copy number of target nucleic acid molecules present in the clonal object as discussed in Logan et al. Real-Time PCR: Current Technology and Applications, Caister Academic Press. (2009). RT-PCR follows the general principle of polymerase chain reaction, however inclusion of detection molecules, such as non-specific fluorescent dyes that intercalate with any double-stranded DNA, or sequence-specific DNA probes consisting of oligonucleotides that are labeled with a fluorescent reporter, which permits detection only after hybridization of the probe with its complementary DNA target, allows for the detection of nucleic acid formed during amplification. The rate of detectable molecules is proportional to the copy number of target nucleic acid molecules present in the clonal object. Furthermore, quantifying the target nucleic acid molecule or amplicons can be done following amplification using standard gel electrophoresis and/or Southern blot techniques, which are well known in the art.

In embodiments, the method further includes sequencing the amplification product(s). Sequencing includes, for example, detecting a sequence of signals within the particle. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the readout is accomplished by epifluorescence imaging. A variety of sequencing chemistries are available, non-limiting examples of which are described herein.

In embodiments of the methods provided herein, the target polynucleotides are at a concentration in the sample selected to produce amplicon clusters having a desired density. For example, the concentration of target polynucleotides is selected based on a calculation of (a) the average size of a cluster of amplicons that will result from amplification under selected conditions (e.g., a selected duration and number of extension steps), and (b) a desired separation between adjacent amplicon clusters in the array.

In embodiments of the methods provided herein, the amplicon clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, 5.0 μm or a number or a range between any two of these values. The mean or median separation may be measured center-to-center (i.e., the center of one amplicon cluster to the center of a second amplicon cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

Neighboring features of an array can be discrete one from the other in that they do not overlap. Accordingly, the features can be adjacent to each other or separated by a gap (e.g., an interstitial space). In embodiments where features are spaced apart, neighboring sites can be separated, for example, by a distance of less than 10 μm, 5 μm, 1 μm, 0.9 μm, 0.8 μm, 0.7 μm, 0.6 μm, 0.5 μm or less. The layout of features on an array can also be understood in terms of center-to-center distances between neighboring features. An array useful in the invention can have neighboring features with center-to-center spacing of less than about 10 μm, 5 μm, 1 μm, 0.9 μm, 0.8 μm, 0.7 μm, 0.6 μm, 0.5 μm or less. Furthermore, it will be understood that the distance values described above and elsewhere herein can represent an average distance between neighboring features of an array. As such, not all neighboring features need to fall in the specified range unless specifically indicated to the contrary, for example, by a specific statement that the distance constitutes a threshold distance between all neighboring features of an array.

In embodiments, each feature generated on the surface of an array can be of similar or smaller size than the area of the surface occupied by the particle from which the feature was produced, and all the features will typically be of similar size and intensity to each other. The uniform size, uniform intensity, and lack of overlap provides a convenient density of features per unit area. Detection of tightly packed non-overlapping arrays with features of uniform size and intensity are typically easier to analyze than images where a subset of the features overlap with each other.

In embodiments of the methods provided herein, the amplicon clusters have a mean or median diameter of about 100-2000 nm, or about 200-1000 nm. In embodiments, the mean or median diameter is about 100-3000 nanometers, about 500-2500 nanometers, about 1000-2000 nanometers, or a number or a range between any two of these values. In embodiments, the mean or median diameter is about or at most about 100, 200, 300, 400, 500, 600, 700, 800, 900, 1000, 1100, 1200, 1300, 1400, 1500, 1600, 1700, 1800, 1900, 2000 nanometers or a number or a range between any two of these values.

In embodiments, the arrays include about 10,000,000 features/cm² to about 5,000,000,000 features/cm². In embodiments, the arrays include about 100,000,000 features/cm² to about 1,000,000,000 features/cm². In embodiments, the arrays include about 100,000 features/cm² to about 100,000,000 features/cm². In embodiments, the arrays include about or about 10,000,000 features/cm² to about 50,000,000 features/cm².

In embodiments, the method does not include ligating an adapter to the target polynucleotide. The terms “ligating”, “ligation” and their derivatives refer generally to the process for covalently linking two or more molecules together, for example covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, the ligation can include forming a covalent bond between a 5′ phosphate group of one nucleic acid and a 3′ hydroxyl group of a second nucleic acid thereby forming a ligated nucleic acid molecule.

In an aspect is provided a method of sequencing two or more RNA molecules (i.e., target polynucleotides), the method including: a) contacting the array as described herein with a sample including a plurality of RNA molecules (e.g., a sample including a plurality of different RNA molecules); and b) immobilizing the plurality of RNA molecules (e.g., hybridizing the plurality of RNA molecules to a first plurality of immobilized oligonucleotides and extending the immobilized oligonucleotides with a reverse transcriptase), thereby generating a plurality of immobilized complements of the plurality of RNA molecules. In embodiments, the method further includes: c) annealing the immobilized complements of the plurality of RNA molecules to a second plurality of immobilized oligonucleotides; and d) extending the second plurality of immobilized oligonucleotides with a DNA polymerase thereby generating a plurality of immobilized target polynucleotides. In embodiments, the method further includes further includes repeating steps a) to d), thereby amplifying the plurality of RNA molecules. In embodiments, the method further includes contacting the immobilized polynucleotides with a sequencing primer, extending the sequencing primer to incorporate a detectable label that indicates the identity of a nucleotide in the target polynucleotides, detecting the detectable label, and repeating the extending and detecting steps, thereby sequencing the two or more RNA molecules.

In an aspect is provided, a method of sequencing two or more cfDNA molecules (i.e., target polynucleotides), the method including: a) contacting the array as described herein with a sample including a plurality of cfDNA molecules (e.g., a sample including a plurality of different cfDNA molecules); and b) immobilizing the plurality of cfDNA molecules (e.g., hybridizing the plurality of cfDNA molecules to a first plurality of immobilized oligonucleotides and extending the immobilized oligonucleotides), thereby generating a plurality of immobilized complements of the plurality of cfDNA molecules. In embodiments, the method further includes: c) annealing the immobilized complements to a second plurality of immobilized oligonucleotides; and d) extending the second plurality of immobilized oligonucleotides thereby generating a plurality of immobilized target polynucleotides. In embodiments, the method further includes further includes repeating steps a) to d), thereby amplifying the plurality of cfDNA molecules. In embodiments, the method further includes contacting the immobilized polynucleotides with a sequencing primer, extending the sequencing primer to incorporate a detectable label that indicates the identity of a nucleotide in the target polynucleotides, detecting the detectable label, and repeating the extending and detecting steps, thereby sequencing the two or more cfDNA molecules.

In an aspect is provided, a method of sequencing two or more V, D, and/or J segment DNA molecules (i.e., target polynucleotides), the method including: a) contacting the array as described herein with a sample including a plurality of V, D, and/or J segment DNA molecules (e.g., a sample including a plurality of different V, D, and/or J segment DNA molecules); and b) immobilizing the plurality of V, D, and/or J segment DNA molecules (e.g., hybridizing the plurality of V, D, and/or J segment DNA molecules to a first plurality of immobilized oligonucleotides and extending the immobilized oligonucleotides), thereby generating a plurality of immobilized complement polynucleotides. In embodiments, the method further includes: c) annealing the plurality of immobilized complements to a second plurality of immobilized oligonucleotides; and d) extending the second plurality of immobilized oligonucleotides thereby generating a plurality of immobilized target polynucleotides. In embodiments, the method further includes further includes repeating steps a) to d), thereby amplifying the plurality of V, D, and/or J segment DNA molecules. In embodiments, the method further includes contacting the immobilized polynucleotides with a sequencing primer, extending the sequencing primer to incorporate a detectable label that indicates the identity of a nucleotide in the target polynucleotides, detecting the detectable label, and repeating the extending and detecting steps, thereby sequencing the two or more V, D, and/or J segment DNA molecules.

In an aspect is provided a method of detecting cancer in a sample. In embodiments, the method includes contacting the array as described herein with a sample including a plurality of polynucleotides and immobilizing the plurality of polynucleotides (e.g., hybridizing the plurality of polynucleotide molecules to a first plurality of immobilized oligonucleotides and extending the immobilized oligonucleotides), thereby generating a plurality of immobilized complements of the plurality of polynucleotide molecules. In embodiments, the method includes amplifying and sequencing the polynucleotides, and detecting cancer when one or more of the polynucleotides includes a cancer-associated gene or cancer-associated biomarker.

A sample may include material obtained from cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

### III. COMPOSITION AND KITS

In an aspect is provided a composition including a first primer and a second primer as described herein for the capture and detection of a target polynucleotide. In embodiments, the target polynucleotide includes a first endogenous region and a second endogenous region as described herein. In embodiments, the first primer includes a first target binding sequence to the target polynucleotide. In embodiments, the first primer includes a first target binding sequence to the first endogenous region of the target polynucleotide. In embodiments, the second primer includes a second target binding sequence to the target polynucleotide. In embodiments, the second primer includes a second target binding sequence to the complement of the second endogenous region of the target polynucleotide. In embodiments, the composition further includes a solid support as described herein. In embodiments, the solid support is a flow cell as described herein. In embodiments, detection of a target polynucleotide includes sequencing.

In an aspect, provided herein are kits for use in accordance with any of the methods disclosed herein, and including one or more elements thereof. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes a solid support, where each well already includes a third primer and fourth primer attached to the solid support. In embodiments, the solid support is in a container. In embodiments, the solid support is a flow cell. The container may be a storage device or other readily usable vessel capable of storing and protecting the solid support.

In embodiments, the kit includes a first primer as described herein. In embodiments, the kit includes a second primer as described. In embodiments, the kit includes a plurality of the first primer as described herein. In embodiments, the kit includes a plurality of the second primer as described herein.

In embodiments, the kit includes a polymerase capable of extending a primer as described herein. In embodiments, the kit includes a polymerase capable of extending the first primer as described herein. In embodiments, the kit includes a polymerase capable of extending the second primer as described herein. In embodiments, the kit includes a polymerase capable of extending the third primer as described herein. In embodiments, the kit includes a polymerase, wherein the polymerase is a strand-displacing or non-strand displacing polymerase. In embodiments, the polymerase is a strand-displacing polymerase. In embodiments, the strand-displacing polymerase is Bst large fragment (Bst LF) polymerase, Bst 3.0 polymerase, Bst2.0 polymerase, Bsu polymerase, SD polymerase, Vent exo-polymerase, Phi29 polymerase, or a mutant thereof. In embodiments, the polymerase is Bst DNA Polymerase, Vent (exo-) DNA Polymerase, Pfu DNA polymerase, Taq polymerase, Phusion High-Fidelity DNA Polymerase, Q5 High-Fidelity DNA Polymerase, or mutant of any one of the foregoing. In embodiments, the polymerase is Bst DNA Polymerase, Vent (exo-) DNA Polymerase, Phusion High-Fidelity DNA Polymerase, or Q5 High-Fidelity DNA Polymerase. In embodiments, the polymerase is a Pyrococcus polymerase (e.g., a polymerase described in WO 2018/148723 or WO 2020/056044, each of which are incorporated herein by reference for all purposes). In embodiments, the polymerase is a Bst DNA polymerase (e.g., exonuclease minus Bst), phi29 DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In embodiments, the polymerase is a phi29 DNA polymerase wild type phi29 DNA polymerase (e.g., MagniPhi from Expedeon), or variant EquiPhi29 DNA polymerase (e.g., from Thermo Fisher Scientific), or chimeric QualiPhi DNA polymerase (e.g., from 4basebio). In embodiments, the polymerase is a non-strand displacing polymerase. In embodiments, the non-strand displacing polymerase is T4 DNA polymerase. In embodiments, the non-strand displacing polymerase is T7 DNA polymerase.

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator™ γ, 9°N polymerase (exo-), Therminator™ II, Therminator™ III, or Therminator™ IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, kits described herein include labeled nucleotides including four differently labeled nucleotides, where the label identifies the type of nucleotide. For example, each of an adenine nucleotide, or analog thereof; a thymine nucleotide; a cytosine nucleotide, or analog thereof; and a guanine nucleotide, or analog thereof may be labelled with a different fluorescent label, or a different combination of labels. In embodiments, the adenine nucleotide, or analog thereof, a thymine nucleotide; a cytosine nucleotide, or analog thereof; and a guanine nucleotide, or analog thereof may be labelled with a different fluorescent label (or different combination of labels) and one may unlabeled.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a sub-portion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. In embodiments, the kit includes, without limitation, nucleic acid primers, probes, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

### EXAMPLES

### Example 1. Onboard Amplification

In the realm of next-generation sequencing (NGS), the preparation of the sequencing library is a pivotal step that precedes the actual sequencing process. This preparation involves processing the nucleic acid samples, either DNA or RNA, to generate a collection of library molecules that are suitable for sequencing. Key to this process is the addition of functional sequences, often referred to as adapters, to the ends of these fragments. For instance, Singular Genomics® systems utilize specific sequences (S1 or S2), whereas the Illumina® platforms employ the P5 and P7 sequences. The flow cell for each respective platform includes immobilized complementary oligonucleotides for capture and amplification. The functional sequences are crucial for the subsequent steps of the sequencing workflow, including the amplification, purification, and loading of the library onto a flow cell for sequencing.

Traditional approaches to library preparation demands meticulous manual intervention. Researchers first need to prepare the library by enzymatically or mechanically fragmenting the nucleic acids, attaching the necessary adapters, and then purifying the library to ensure that it is free from contaminants and ready for sequencing. This sequence of actions not only requires a significant investment of time and resources but also introduces potential points of error, as each step must be carefully controlled to ensure the integrity and quality of the library. Prior to this disclosure, adapter ligation has been a critical step in the library prep workflow, where poor ligation efficiency across nucleic acid species dramatically skews sequencing results. Some commercial library prep kits have been reported to have very low (<15%) ligation efficiency, while different kits were found to have high sample loss during preparation (>50%) (see, e.g., Aigrain L et al. BMC Genomics. 2016; 17:458, which is incorporated herein by reference in its entirety). Commercial library prep kits for targeted sequencing have also displayed significant variability with regards to library complexity, coverage uniformity, and errors in the UMIs (see, e.g., Chung J et al. BMC Genomics. 2019; 20 (1); 216, which is hereby incorporated by reference in its entirety).

Conventional methods of library preparation poses several challenges. Firstly, the requirement for multiple manual steps increases the risk of introducing errors and variability, which can affect the reliability of the sequencing results. Secondly, the process is time-consuming, often taking several hours to complete, which limits the throughput and efficiency of sequencing projects. Furthermore, the necessity for specialized equipment and skilled personnel for library preparation can be a significant barrier, particularly for smaller laboratories or those with limited resources.

To address these and other challenges in the art, we developed an innovative method that simplifies the library preparation process by enabling the addition of functional sequences directly on or within the sequencing instrument. This novel approach enables researchers to bypass the traditional, labor-intensive steps of library preparation. Instead, nucleic acid molecules can be directly added to the instrument, where the functional sequences are incorporated as part of the automated sequencing workflow. This method significantly reduces the hands-on time required for library preparation, minimizing the potential for human error and increasing the efficiency of the sequencing process. By integrating the addition of functional sequences into the sequencing instrument's operation, this solution streamlines the workflow, enabling faster turnaround times and higher throughput. Furthermore, it democratizes access to high-throughput sequencing by reducing the technical barriers and resources required for library preparation, making it more accessible to a broader range of researchers and institutions.

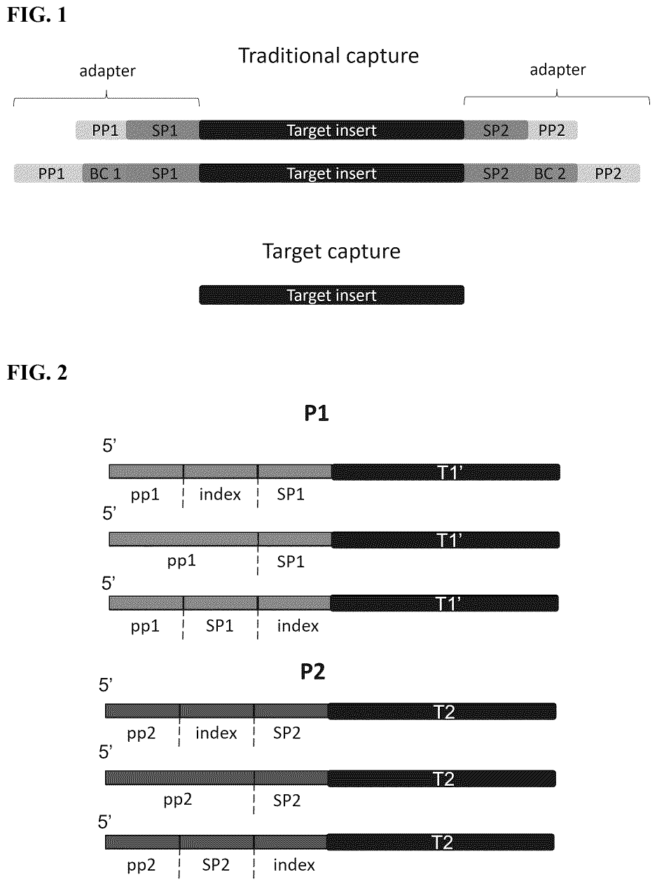

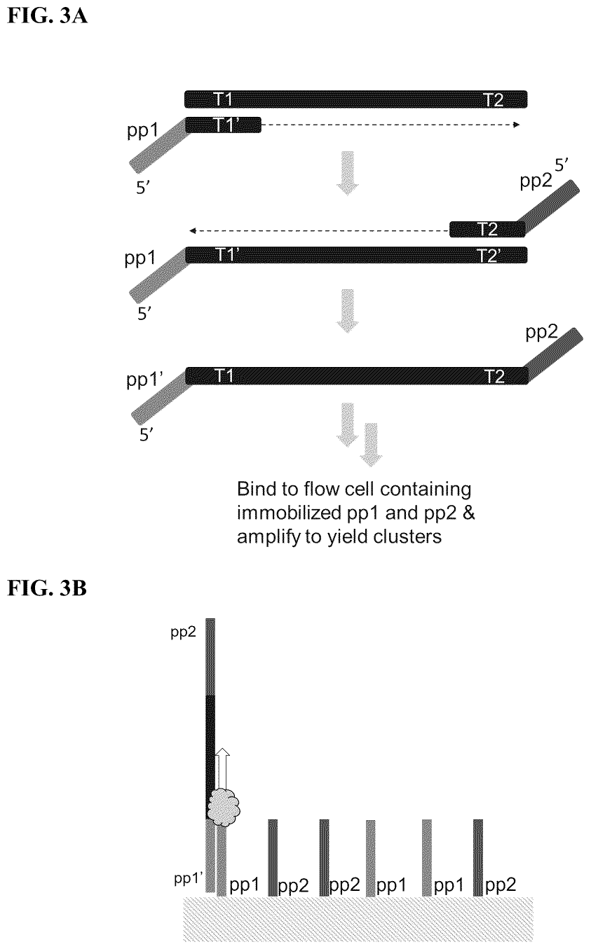

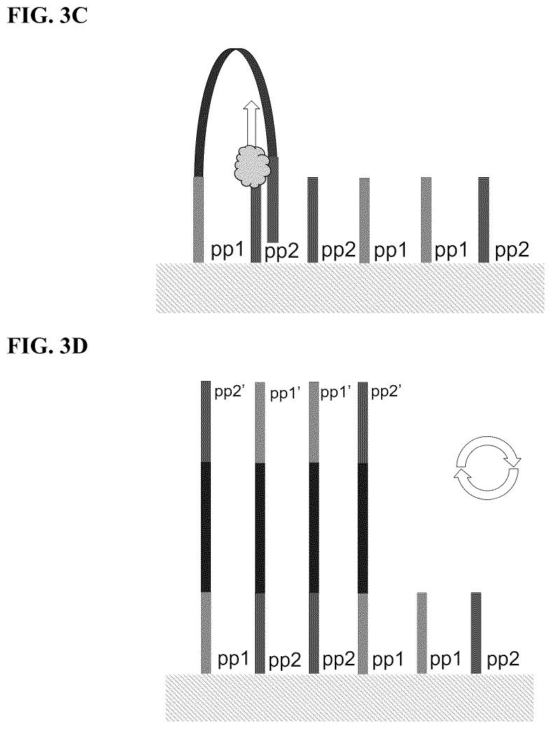

Described herein is a method of nucleic acid sequencing that reduces sample loss by bypassing the typical library preparation workflow. Briefly, the method includes introducing a nucleic acid molecule of interest (i.e., target molecule) into the flow cell. Contrary to conventional sequencing workflows, where the target insert requires adapter ligation to render it usable on commercial sequencing platforms (FIG. 1), the nucleic acid molecule does not contain functional sequences to enable hybridization to the flow cell using methods described herein. Rather, solution-phase primer oligonucleotides including a sequence complementary to the target molecule and a sequence complementary to an immobilized oligonucleotide (e.g., S1) are introduced and subsequently amplified with PCR, molecules with functional sequences on the ends are obtained (FIG. 3A and FIG. 4). FIG. 2 shows examples of the primer oligonucleotide sequences, referred to as P1 and P2, that include sequences that hybridize with the immobilized oligonucleotides, S1 and S2, respectively. The P1 primer contains a first platform primer sequence (pp1), which is a sequence complementary to a first immobilized primer (e.g., an oligonucleotide attached to a solid support), an optional index sequence, a first sequencing primer (SP1) sequence, and a first target hybridization sequence (T1′). The P2 primer contains a second platform primer sequence (pp2), which is a sequence complementary to a second immobilized primer, an optional index sequence, and a second sequencing primer (SP2) sequence, and a second target hybridization sequence (T2).

Within the flow cell, a first primer oligonucleotide (e.g., a P1 oligonucleotide illustrated in FIG. 2) binds to a target molecule. The first primer oligonucleotide is extended with a polymerase to provide a complement of the original target molecule with the pp1 sequence attached on the 5′ end. A second primer oligonucleotide (e.g., a P2 oligonucleotide illustrated in FIG. 2) binds to the complement target molecule and is extended with a polymerase to provide a nucleic acid molecule including both pp1′ and pp2 at the ends, referred to herein as a library molecule. The library molecules may be subjected to additional rounds of solution-phase amplification. Subsequent binding and extension (FIG. 3B) immobilizes the library molecule(s) to a flowcell containing immobilized platform primers, generating a complement of the library molecule. FIG. 3C depicts a first solid-phase amplification cycle, whereby the immobilized complement molecule anneals to immobilized oligonucleotide and is extended to generate immobilized sample polynucleotides. This solid phase amplification process is repeated (FIG. 3D) to yield features containing a plurality of immobilized sample polynucleotides.

FIG. 4 provides an example of the onboard amplification methods described herein. Minimally processed nucleic acid fragments are introduced into a flow cell. Solution phase primers are introduced and following cycles of amplification (e.g., 2-20 cycles), the nucleic acid fragments include functional sequences capable of binding to the immobilized nucleotides in the flow cell. Subsequent cycles of solid-phase amplification provide monoclonal clusters (i.e., features) of amplification products, which may be detected (e.g., sequenced).

### Example 2. Rare-Target Quantification

It is a major challenge to distinguish true variants from background noise for rare variant discovery. Identification and tracking of cancer-causing mutations via liquid biopsy, for example, are increasingly being used for measuring potential therapeutic response, quantifying residual tumor burden, and studying resistance to potential targeted therapies. However, detecting circulating tumor DNA for liquid biopsy applications is challenging because the targets of interest are only a small fraction of the total circulating cell-free nucleic acids collected in the sample. Liquid biopsy has therefore relied on highly sensitive, and complex, assays (e.g., digital PCR) to detect low-abundant quantities of highly fragmented nucleic acids, such as those derived from tumors. Typically, large amounts of sequencing data (high sequencing depth) are required to differentiate a true variant from an amplification and/or sequencing error using previous methods. In addition, the heterogeneity of mutations within populations of rare cells in liquid biopsy samples, such as plasma or saliva, make it difficult to distinguish between sequencing-related errors and true somatic mutations originating from tumors. Methods and constructs provided herein do not necessarily require such large sequencing depths, thereby dramatically reducing the costs associated with sequencing.

Digital polymerase chain reaction (dPCR) is a refinement of conventional PCR methods that can be used to directly quantify and clonally amplify nucleic acids. Conventional PCR assumes that amplification is exponential. Therefore, nucleic acids may be quantified by comparing the number of amplification cycles and amount of PCR end-product to those of a reference sample. Digital PCR is designed to quantitate target nucleic acid sequences by dividing the sample into many independent partitions such that each contains either a few or no target sequences. Typically, the accuracy and more importantly the precision of determination by dPCR may be improved by using a greater number of partitions. For example, dPCR may be carried out identically in an array having between at least 100-100,000 partitions, e.g., between at least 1,000-100,000 reaction sites, or between at least 10,000-100,000 reaction sites. The capture or isolation of individual nucleic acid molecules can be performed in micro well plates, capillaries, the dispersed phase of an emulsion, and arrays of miniaturized chambers, as well as on nucleic acid binding surfaces. For example, the Applied Biosystems™ QuantStudio™ Absolute Q™ Digital PCR System is a plate-based digital PCR (dPCR) platform that can run up to 16 samples per run, with 20,480 micro-chambers per reaction. Individual dPCR assays for performing liquid biopsy analysis of rare cancer variants are commercially available, but are limited to detecting only a few targets in a single reaction. For example, the Absolute Q™ Liquid Biopsy Digital PCR Assays offering from Thermo Fisher can only detect up to four targets in a single reaction, increasing the number of reactions and reagent expense required to screen a broad panel of variants. In contrast, the compositions and methods described herein can target greater than 100 or greater than 1000 unique targets using a sequencing-based readout, with at least 2×10⁶ (384-well plate) or 1×10⁷ (96-well plate) reaction sites, enabling detection of rare variants with a sensitivity of <0.01%, compared to commercial dPCR offerings that have on average a 0.1% sensitivity.

While the methods described herein can be applied to DNA extracted directly from tumor cells, studying cell-free DNA (cfDNA) proves to be a useful test case for testing the capabilities of the linked duplex sequencing methods described herein, for recovering sequencing depth and for detecting rare mutations in a clinical application. Liquid biopsies utilize cell-free DNA (cfDNA) that is derived from cells, both normal and cancerous, that have undergone apoptosis or necrosis and released their DNA contents into their environment (see, e.g., Chu D and Park B H. Virchows Arch. 2017; 471 (2): 147-154). The component of cfDNA that is released from cancer cells is more commonly referred to as circulating tumor DNA, or ctDNA. Nucleic acids, e.g., cfDNA, are released into the bloodstream and other body fluids as part of natural cell apoptosis, necrosis, and secretion, and includes both single- and double-stranded DNA fragments that are relatively short (overwhelmingly less than 200 base-pairs) and are normally at a low concentration (e.g. 1-100 ng/mL in plasma). It is known that the concentration of cfDNA and ctDNA in plasma correlates with tumor size and stage. For example, patients having stage I cancer types had fewer than 10 copies per 5 ml of tumor mutations in plasma. In contrast, the copy number increased 10 to 100 times among late-stage patients (Haque et al. bioRxiv. 2017; 237578.). Thus, ctDNA assays used for early cancer diagnosis should be highly sensitive. Commercial sequencing solutions require UMIs on both strands of the double-stranded template, followed by low-error sequencing. To determine a true variant using previous commercial solutions, large amounts of sequencing data (high sequencing depth) is required to generate a consensus sequencing read to confidently ascertain a single nucleotide change.

Recent cancer genome sequencing studies have shown that virtually all cancers harbor somatic genetic alterations. These alterations include insertions, deletions, single-base substitutions, and translocations (Vogelstein et al Science. 2013 Mar. 29; 339 (6127): 1546-1558). In cancer, a proportion of cfDNA circulating in plasma can come from the tumor, with the relative contribution of cfDNA coming from the tumor increases with cancer severity. The rate of these chromosomal changes in cancer cells is elevated and mutations can be challenging to detect accurately (Pietrasz et al Clin Cancer Res. 2017 Jan. 1; 23 (1): 116-123). While typical commercial sequencing instruments have a sequencing error rate that varies from about 0.05-1% (Quail et al Nat Methods. 2008 December; 5 (12): 1005-10), and can reveal comprehensive genomic alterations, it remains a challenge to distinguish variants at such low fraction from background errors of sequencing. Nonetheless, identifying cfDNA harboring these genetic alterations serves as valuable biomarkers and accurately detecting these variants will significantly improve current methods of cancer diagnosis, cancer progression monitoring, therapy effectiveness, and early-stage detection.

Described herein is a method of targeted nucleic acid sequencing that enables multiplexed detection of cfDNA genetic alterations and provides an alternate way to obtain digital quantification of original target nucleic acids in a sample while at the same time obtaining sequence information, without the need to perform any compartmentalization as is required by existing digital PCR offerings.

The method described herein could be applied to detect and analyze cfDNA in blood samples. Due to the small amount of cfDNA present in most blood samples, whole genome amplification (WGA) may be used to obtain adequate amount of nucleic acids suitable for the assay. cfDNA can be circularized and RCA can be used to obtain large DNA fragments suitable for WGA. Typically, the plurality of cfDNA molecules is fragmented, end-repaired, and applied to a solid support with immobilized primers, wherein one primer type is complementary to a first sequence of the nucleic acid fragment, whereas the second primer type is homologous to a second sequence of the fragment. The method described herein significantly minimizes library preparation time as fragmentation, end-repairing, and adapter ligation is not required. A workflow implementing the method described herein is as follows: a blood sample is obtained from a patient, and cfDNA is isolated using methods known in the art. Subsequently, the plurality of cfDNA molecules is applied to a standard flow cell (e.g., the F2 or F3 flow cell from Singular Genomics) in the presence of primers capable of hybridizing with target sequences of the target polynucleotide(s) and capable of hybridizing with the surface immobilized primers on a standard flow cell. As shown in FIG. 3A, each primer harbors a sequence that targets an endogenous gene region within target polynucleotide sequence (referred to as T1′ and T2) to enable the capture of the target polynucleotide using two endogenous regions of the target polynucleotide. In addition, each primer also includes a sequence that does not hybridize to the target polynucleotide but used for hybridizing with surface-immobilized oligonucleotides on the surface of flow cells, which are shown in FIGS. 3A and 3B as pp1 and pp2. In the presence of a polymerase and nucleotides, the immobilized oligonucleotide is extended to generate a complement of a portion of the captured fragment. Following the immobilization of the target polynucleotides onto the flow cell, bridge amplification can be performed directly on the flow cell to generate clusters for sequencing (FIGS. 3C, 3D, and 4).

Exemplary target binding sequences in the primers for use with the methods described herein are listed in Table 1. The corresponding gene of interest, amino acid (A.A.) mutations, and nucleotide sequence (CDS) mutations are also provided. The CDS mutations are provided in standard mutation nomenclature in molecular diagnostics, provided by the Human Genome Variation Society; see for example Ogino S, et. al. J Mol Diagn. 2007 February; 9 (1): 1-6. Note that more than one set of mutations for a given gene may be targeted by a given primer pair, as indicated in Table 1 herein. For example, the primer pair of SEQ ID NO:7 and SEQ ID NO:8 allow for identification of mutations at nucleotide positions 181 and 182 of the NRAS gene. Each target binding sequence in Table 1 was designed to be approximately 40 nucleotides in length (range of about 35 to about 50 nucleotides in length). The 3′ end of a target binding sequence (e.g., the T2 oligonucleotide) was designed to be complementary to about 1 to less than about 20 nucleotides away from the target mutation site. The 3′ ends of the T1 and T2 oligonucleotide pairs were designed to be complementary to regions separated by approximately 100 nucleotides (range of 80 to 120 nucleotides), preferably without complete complementarity for 1 to 3 bases at the 3′ end of each of the target sequences. The sequences were designed to have minimal self-folding, and for the oligonucleotides of each pair to have less than or equal to 5 °C difference in melting temperature.

TABLE 1
Example primer sets for capturing genes of interest.

| | | | Primer Pair Set | |
|---|---|---|---|---|
| | | | First | Second |
| | | | Target | Target |
| | | | Binding | Binding |
| Gene | A.A. Mutation(s) | CDS Mutation(s) | Sequence | Sequence |
| NRAS | p.Q61K | c.180_181AC > TA; | SEQ ID | SEQ ID |
| | p.Q61E | c.181C > A | NO: 7 | NO: 8 |
| | p.Q61P | c.181C > G | | |
| | p.Q61L | c.182A > C | | |
| | p.Q61R | c.182A > T | | |
| | | c.182A > G | | |
| NRAS | p.G12S | c.34G > A | SEQ ID | SEQ ID |
| | p.G12D | c.35G > A | NO: 9 | NO: 10 |
| | p.G12A | c.35G > C | | |
| | p.G12V | c.35G > T | | |
| | p.G13R | c.37G > C | | |
| | p.G13C | c.37G > T | | |
| | p.G13= | c.39T > C | | |
| MLF2 | p.R158W | c.472C > T | SEQ ID | SEQ ID |
| | | | NO: 11 | NO: 12 |
| KRAS | p.A146T | c.436G > T | SEQ ID | SEQ ID |
| | p.A146V | c.437C > T | NO: 13 | NO: 14 |
| | p.A146P | c.436G > C | | |
| KRAS | p.K117N | c.351A > T | SEQ ID | SEQ ID |
| | | | NO: 15 | NO: 16 |
| KRAS | p.Q61R | c.182_183AA > GT; | SEQ ID | SEQ ID |
| | p.Q61K | c.182A > G | NO: 17 | NO: 18 |
| | p.Q61L | c. 181C > A | | |
| | p.Q61H | c.182A > T | | |
| | | c. 183A > C; | | |
| | | C.183A > T | | |
| KRAS | p.G12C | c.34G > T | SEQ ID | SEQ ID |
| | p.G12S | c.34G > A | NO: 19 | NO: 20 |
| | p.G12R | c.34G > C | | |
| | p.G12V | c.35G > T | | |
| | p.G12D | c.35G > A | | |
| | p.G13C | c.37G > T | | |
| | p.G13S | c.37G > A | | |
| | p.G13R | c.37G > C | | |
| TP53 | p.R342* | c.1024C > T | SEQ ID | SEQ ID |
| | | | NO: 21 | NO: 22 |
| TP53 | p.R282W | c.844C > T | SEQ ID | SEQ ID |
| | | | NO: 23 | NO: 24 |
| TP53 | p.R282W | c.844C > T | SEQ ID | SEQ ID |
| | | | NO: 25 | NO: 26 |
| TP53 | p.R273H | c.818G > A | SEQ ID | SEQ ID |
| | p.R273L | c.818G > T | NO: 27 | NO: 28 |
| TP53 | p.R248W | c.742C > T; | SEQ ID | SEQ ID |
| | p.R248Q | c.741_742CC >> TT | NO: 29 | NO: 30 |
| | p.R248L | c.743G > A; | | |
| | p.G245C | c.743_744GG >> AA | | |
| | p.G245S | c.743G > T | | |
| | | c.733G > T | | |
| | | c.733G > A | | |
| TP53 | p.V173L | c.517G > T | SEQ ID | SEQ ID |
| | | | NO: 31 | NO: 32 |
| ERBB2 | p.L755S | c.2264T > C | SEQ ID | SEQ ID |
| | | | NO: 33 | NO: 34 |
| ERBB2 | p.V777L | c.2329G > T | SEQ ID | SEQ ID |
| | | | NO: 35 | NO: 36 |
| IDH1 | p.R132C | c.394C > T | SEQ ID | SEQ ID |
| | | | NO: 37 | NO: 38 |
| PIK3CA | p.M1043V | c.3127A > G | SEQ ID | SEQ ID |
| | p.M1043I | c.3129G > T | NO: 39 | NO: 40 |
| | p.H1047R | c.3140A > G | | |
| | p.H1047L | c.3140A > T | | |
| KIT | p.D816V | c.2447A > T | SEQ ID | SEQ ID |
| | p.D816E | c.2448C > G | NO: 41 | NO: 42 |
| PIK3R1 | p.K567E | c.1699A > G | SEQ ID | SEQ ID |
| | | | NO: 43 | NO: 44 |
| NPM1 | p.W288fs*12 | c.860_863dup TCTG | SEQ ID | SEQ ID |
| | | | NO: 45 | NO: 46 |
| EGFR | p.S492R | c.1474A > C; | SEQ ID | SEQ ID |
| | | c.1476C > A | NO: 47 | NO: 48 |
| EGFR | p.G719C | c.2155G > T | SEQ ID | SEQ ID |
| | p.G719S | c.2155G > A | NO: 49 | NO: 50 |
| EGFR | p.E746_S752 > A | c.2237_2254del18 | SEQ ID | SEQ ID |
| | p.E746_T751delEL | c.2240_2254del15 | NO: 51 | NO: 52 |
| | REAT | c.2240_2257del18 | | |
| | p.L747_P753 > S | c.2239_2248TTAAGAGAA | | |
| | p.L747_A750 > P | G > C | | |
| | p.L747_T751 > P | c.2239_2251 > C | | |
| | p.E746_S752 > V | c.2237_2255 > T | | |
| | p.L747_P753delins | c.2239_2258delinsCA | | |
| | Q | c.2238_2252 > GCA | | |
| | p.L747_T751 > Q | c.2238_2248 > GC | | |
| | p.L747_A750 > P | c.2237_2251del15 | | |
| | p.E746_T751delins | c.2236_2253del18 | | |
| | A | c.2235_2252 > AAT | | |
| | p.E746_T751delEL | c.2238_2252del15 | | |
| | REAT | c.2240_2251del12 | | |
| | p.E746_T751delins | c.2238_2255del18 | | |
| | I | c.2235_2249del15; | | |
| | p.L747_T751delLR | c.2236_2250del15 | | |
| | EAT | c.2239_2256del18 | | |
| | p.L747_T751 > S | | | |
| | p.E746_S752 > D | | | |
| | p.E746_A750delEL | | | |
| | REA | | | |
| | p.L747_S752delLR | | | |
| | EATS | | | |
| EGFR | p.D770_N771insG | c.2310_2311insGGT | SEQ ID | SEQ ID |
| | | | NO: 53 | NO: 54 |
| EGFR | p.L858R | c.2573T > G | SEQ ID | SEQ ID |
| | p.L861Q | c.2582T > A | NO: 55 | NO: 56 |
| BRAF | p.V600K | c.1798_1799GT > AA | SEQ ID | SEQ ID |
| | p.V600E | c.1799_1800TG > AA; | NO: 57 | NO: 58 |
| | | c.1799T > A | | |
| JAK2 | p.V617F | c.1849G > T | SEQ ID | SEQ ID |
| | | | NO: 59 | NO: 60 |
| NPM1 | p.W288fs*12 | c.860_863dup TCTG | SEQ ID | SEQ ID |
| | | | NO: 59 | NO: 60 |

For example, a pair of primers can be designed to detect the HER2 gene and the reference gene RPP30 is provided (for additional information, see, Yang S R et al. Mod. Pathol. 2020; 33:1546-56, which is incorporated herein by reference in its entirety), wherein the primer pair targets two endogenous regions within the HER2 gene. As shown in FIG. 4, the target polynucleotide is applied directly to the flow cell in the presence of a first primer and a second primer without any prior adapter ligation step. Depicted in FIG. 3A, the first primer harbors a sequence capable of hybridizing with a surface-immobilized primer on a standard flow cell (pp1) and a first target binding sequence (T1′). The second primer harbors a sequence capable of 10 hybridizing with a surface-immobilized primer on a standard flow cell (pp2) and a second target binding sequence (T2) (FIG. 3A). Following the hybridization of the first primer to the first endogenous sequence within the HER2 gene, the first primer is extended with a polymerase to generate a sequence including the pp1 sequence and a first complement of the target polynucleotide sequence, which includes the complement of T1 and T2 (e.g., T1′ and T2′, respectively). Hybridization of the second primer onto the first complement of the target polynucleotide ensues and extension of the second primer generates a second complement sequence, which includes the pp1 sequence, the target sequence with T1 and T2, and the pp2 sequence. The presence of the pp1 and pp2 enables hybridization and immobilization of the target polynucleotide sequences to a standard flow cell. Following hybridization and immobilization of the targeted panel of DNA fragments, solid phase amplification (e.g., bridge PCR (bPCR) or RCA) is performed (see, FIGS. 3C and 3D). During the bPCR cycle, the immobilized extended complement hybridizes to the second type of immobilized oligonucleotide. In the presence of a polymerase (depicted as the cloud) and nucleotides, the second type of immobilized oligonucleotide is extended to generate an immobilized portion of the captured fragment. The process is repeated under suitable conditions to allow multiple polynucleotide fragments to hybridize to a complementary sequence of the surface immobilized oligonucleotides and to be extended to generate copies of the captured fragment and complements thereof. Following a plurality of bridge PCR amplification cycles, each surface immobilized oligonucleotide that captured a polynucleotide includes a cluster of polynucleotides. In embodiments, a purification step following PCR amplification is not required. The cluster of polynucleotides can then be subjected to sequencing, for example, sequencing-by-synthesis. A plurality of sequencing cycles then occurs, wherein each cycle includes extension and detection of an incorporated nucleotide. The entire array may be selectively sequenced by choosing the appropriate initiator, i.e., the appropriate sequencing primer.

Alternatively, or in addition to multiplexed detection of cfDNA genetic alterations, the method described herein can be applied to sequence targeted panels for cfDNA oncogene sequencing, including targeted cancer exome gene panels, and increase the quality and efficiency of clinical diagnostics. In embodiments, the target polynucleotide includes complements to exon genomic sequences (i.e., nucleic acids encoding for proteins). For example, in tumor exome sequencing, only the coding regions of genes are assessed, which frequently contain mutations that affect tumor progression. Depending on experimental needs, cancer exome sequencing can also be expanded to untranslated regions and microRNA (miRNA) binding sites. For example, an array of primers can be designed to detect a panel of cancer-associated coding sequences and coding sequences for pathogenic variants. Target binding sequences included in primers used for targeted exome sequencing could be designed by leveraging coding sequences from various databases, including RefSeq, Consensus Coding Sequence (CCDS), American College of Medical Genetics and Genomics (ACMG), the Cancer Gene Census from the Catalog of Somatic Mutations in Cancer (COSMIC), and the Online Mendelian Inheritance in Man (OMIM), and ClinVar. For example, primers including target binding sequences for cancer-associated coding sequences, such as p53, KRAS, or PIK3CA, are contemplated (as described in Reda M et al. EBioMedicine. 2020; 51:102624; McCabe M J et al. Sci. Rep. 2019; 9 (1): 17052; Zhou C et al. Ann. Transl. Med. 2021; 9 (18): 1437; and Klevebring D et al. PLOS One. 2014; 9 (8): e104417, each of which is incorporated herein by reference in its entirety). As shown in FIG. 3A, each primer harbors a sequence that targets an endogenous gene region within target polynucleotide sequence (referred to as T1′ and T2) to enable the capture of the target polynucleotide using two endogenous regions of the target polynucleotide. In addition, each primer also includes a sequence that does not hybridize to the target polynucleotide but used for hybridizing with surface-immobilized oligonucleotides on the surface of flow cells, which are shown in FIGS. 3A and 3B as pp1 and pp2. Following the immobilization of the target polynucleotides onto the flow cell, bridge amplification can be performed directly on the flow cell to generate clusters for sequencing (FIGS. 3C, 3D, and 4).

### Example 3. Multiplexed Detection in Low gDNA Input

Next generation sequencing of genomic DNA (gDNA) provides invaluable information as it enables the identification, discovery, and/or analysis of genomic variants present in the intronic regions and exonic regions of DNA to facilitate the identification of clinically actionable genomic targets in a variety of diseases. One common drawback of commercially available library preparation kits is the complexity of the protocols. For example, commercially available DNA Prep with Enrichment from Illumina® facilitates library preparation from gDNA using a workflow that includes 9 steps (tagmentation of genomic DNA, post-tagmentation clean up, amplification of tagmented DNA to generate libraries, post-amplification clean up, pooling of libraries, hybridization of probes, capture of hybridized probes, amplification of captured library, and post-amplification clean up) and requires at least 105 minutes of hands-on time to prepare the library prior to the addition of the library to a flow cell.

Described herein are methods and compositions directed to multiplexed detection of targets, wherein the preparation of the sample library occurs directly on the surface of a flow cell. Specifically, this method enables (1) on-flow cell capture (e.g., immobilization) of target polynucleotides with pairs of primers, wherein each primer in a primer pair includes a sequence complementary to one of two endogenous regions of the target polynucleotide and a sequence complementary to one of two oligonucleotides immobilized onto the surface of a flow cell; and (2) on-flow cell amplification of the captured (e.g., immobilized) target polynucleotides or complements thereof without any purification steps following amplification. The methods described herein could overcome drawbacks stemming from the complexity of commercially available protocols for library preparation as the methods described herein enable the introduction of the target polynucleotides directly onto the flow cell (e.g., direct seeding) without any prior adapter ligation or library preparation step, followed by amplification of the target polynucleotides or complements thereof directly on the surface of the flow cell. This simplification minimizes hands-on time for the user, cost, and the risk for sample loss while increasing efficiency. Additionally, this simplification afforded by the methods described herein is particularly important for target polynucleotides obtained from clinical samples or formalin-fixed paraffin-embedded (FFPE) tissue samples or target polynucleotides obtained in low quantity and/or quality.

A study was performed to directly seed and amplify 25 polynucleotide targets from a library of from wild type human genome, HG004, that was sheared to a fragment size of approximately 500 bp. Primers used to hybridize to the polynucleotide targets included target binding sequences capable of hybridizing to the target polynucleotides (or complements thereof) and sequences capable of hybridizing to the S1/S2′ surface-immobilized oligonucleotides on the surface of commercially available F2 and F3 flow cells from Singular Genomics®. The sequences of the target binding sequences in the primers (e.g., the first or second primers described herein) were provided as oPools™ Oligo Pools from IDT. The sequence of the surface immobilized S1 oligonucleotide is 5′-ACAAAGGCAGCCACGCACTCCTTCCCTGT (SEQ ID NO: 3), and the sequence of the surface immobilized S2′ oligonucleotide is 5′-AGTGGTTGGTGAGGGTCATCTCGCTGGAG (SEQ ID NO:2). Alternatively, the sequence of the first and second primer as described herein could harbor a sequence capable of hybridizing with surface-immobilized oligonucleotides on the surfaces of other flow cells, such as P5/P7 (or complements thereof). The sequence of the surface immobilized P5 oligonucleotide is 5′-AATGATACGGCGACCACCG (SEQ ID NO: 5), and the sequence of the surface immobilized P7 oligonucleotide is 5′-CAAGCAGAAGACGGCATACGA (SEQ ID NO: 6). The methods described herein could be adapted to directly immobilize target polynucleotides to the surface of any commercially available flow cell without any prior adapter ligation step.

Briefly, the protocol used for the multiplexed detection of targets from low input genomic DNA are as follows. To a commercially available F3 flow cell with S1/S2′ surface-immobilized oligonucleotides was added <20 ng of gDNA to each lane and a pool of 25 pairs of primers at a concentration of 10 nM of each primer, wherein each primer harbored a sequence capable of hybridizing with a target polynucleotide sequence and a sequence capable of hybridizing to a surface-immobilized oligonucleotide sequence of the F3 flow cell. The pool of primers and gDNA were allowed to anneal for 2 minutes prior to solution phase amplification for 20 cycles of PCR. During solution phase amplification, the target binding sequences hybridize to one of the two endogenous regions of a target polynucleotide. Hybridization of each primer of a primer pair to the target polynucleotide occurs sequentially in solution, wherein the first primer with the first target binding sequence binds to the target polynucleotide sequence and is extended by a polymerase to generate a first complement of the target polynucleotide template including the first platform binding sequence of the first primer and a complementary sequence of the target polynucleotide template. Following the generation of the first complement of the target polynucleotide template, the second target binding sequence of the second primer binds to the first complement of the target polynucleotide and is extended by a polymerase to generate a second template including the complement of the first platform binding sequence of the first primer, the target polynucleotide sequence, and the second platform binding sequence. Following the solution phase amplification to generate the second template (or plurality thereof), the second template can be immobilized onto the surface of the flow cell by binding to complement of the first platform binding sequence of the first primer to a first surface immobilized oligonucleotide with a complementary sequence (e.g., third primer) to initiate solid phase bridge amplification on the flow cell to enrich the sequences of the immobilized target polynucleotides. The method described herein benefits from directly seeding the target polynucleotide (or plurality thereof) to a standard flow cell without a purification step following amplification. Utilizing this method, single nucleotide variant (SNV) error rates of mutations were obtained at 0.0002%-0.01%.

### SEQUENCES

It is understood that white space, line breaks, and text formatting are not indicative of separate sequences or structural implications. In embodiments, the sequences provided in herein may include a phosphorothioate modification (e.g., at the 3′ end) though not explicitly identified within the sequence.

SEQ ID NO: 7:
5′-CATCCTTTCA GAGAAAATAA TGCTCCTAGT
ACCTGTAGAG
SEQ ID NO: 8:
5′-TATAGATGGT GAAACCTGTT TGTTGGACAT
ACTGGATACA
SEQ ID NO: 9:
5′-ATCCGACAAG TGAGAGACAG GATCAGGTCA GCGGG
SEQ ID NO: 10:
5′-GCTGGTGTGA AATGACTGAG TACAAACTGG
TGGTGGTTG
SEQ ID NO: 11:
5′-GCTGCCAGCC AGGAAGCGGG AGGGAAGGCT CACTC
SEQ ID NO: 12:
5′-GATTCAGACA GTGGACTGGA GCAGATGTCC
ATTGGGCAT
SEQ ID NO: 13:
5′-TTTACTAAAT ATTGTTTTAT TTCCTAGTAT AGCATAATTG
AGAGAAAAAC TGAT
SEQ ID NO: 14:
5′-ACAAAACAGG CTCAGGACTT AGCAAGAAGT
TATGGAATT
SEQ ID NO: 15:
5′-CAGATCTGTA TTTATTTCAG TGTTACTTAC CTGTCTTGTC
TTT
SEQ ID NO: 16:
5′-AAGAGTTAAG GACTCTGAAG ATGTACCTAT GGTCCTAGT
SEQ ID NO: 17:
5′- ATTACTCCTT AATGTCAGCT TATTATATTC
AATTTAAACC CACCT
SEQ ID NO: 18:
5′-AGTAGTAATT GATGGAGAAA CCTGTCTCTT
GGATATTCTC
SEQ ID NO: 19:
5′-TGGTCAGAGA AACCTTTATC TGTATCAAAG
AATGGTCCTG C
SEQ ID NO: 20:
5′-TTTTTATTAT AAGGCCTGCT GAAAATGACT
GAATATAAAC TTGT
SEQ ID NO: 21:
5′-CTTTCCAACC TAGGAAGGCA GGGGAGTAGG GCCAGGAA
SEQ ID NO: 22:
5′-TGTTGCTGCA GATCCGTGGG CGTGAGCGCT TCGAGA
SEQ ID NO: 23:
5′-TCTGAGGCAT AACTGCACCC TTGGTCTCCT CCACCGC
SEQ ID NO: 24:
5′-ACAGCTTTGA GGTGCGTGTT TGTGCCTGTC CTGGGA
SEQ ID NO: 25:
5′-CCTCCACCGC TTCTTGTCCT GCTTGCTTAC CTCGCTT
SEQ ID NO: 26:
5′-AGTAGTGGTA ATCTACTGGG ACGGAACAGC TTTGAGG
SEQ ID NO: 27:
5′-CTCCTCCACC GCTTCTTGTC CTGCTTGCTT ACCTCGC
SEQ ID NO: 28:
5′-CTGAGTAGTG GTAATCTACT GGGACGGAAC AGCTTTGA
SEQ ID NO: 29:
5′-TATGGAAGAA ATCGGTAAGA GGTGGGCCCA GGGGTCAG
SEQ ID NO: 30:
5′-CACCATCCAC TACAACTACA TGTGTAACAG TTCCTGCA
SEQ ID NO: 31:
5′-GGCCAGACCT AAGAGCAATC AGTGAGGAAT CAGAGG
SEQ ID NO: 32:
5′-GCCATGGCCA TCTACAAGCA GTCACAGCAC ATGACG
SEQ ID NO: 33:
5′-CCCCAAACCC CAATGAAGAG AGACCAGAGC CCAGACCT
SEQ ID NO: 34:
5′-CTGGATCCCT GATGGGGAGA ATGTGAAAAT
TCCAGTGGCC
SEQ ID NO: 35:
5′-CTGGGAGCCC AGGCGTCCGC GGTTTTCCCG GACAT
SEQ ID NO: 36:
5′-CTCTCAGCGT ACCCTTGTCC CCAGGAAGCA
TACGTGATGG
SEQ ID NO: 37:
5′-CAATTTCATA CCTTGCTTAA TGGGTGTAGA
TACCAAAAGA TAAGAATAAA AC
SEQ ID NO: 38:
5′-CCCCCGGCTT GTGAGTGGAT GGGTAAAACC TATC
SEQ ID NO: 39:
5′-ATCGGTCTTT GCCTGCTGAG AGTTATTAAC AGTGCAG
SEQ ID NO: 40:
5′-CCTTAGATAA AACTGAGCAA GAGGCTTTGG
AGTATTTCAT G
SEQ ID NO: 41:
5′-GGAAACAATT TTTATCGAAA GTTGAAACTA
AAAATCCTTT GCAGG
SEQ ID NO: 42:
5′-CTCCTTACTC ATGGTCGGAT CACAAAGATT
TGTGATTTTG G
SEQ ID NO: 43:
5′-GTTTTGAAAA GAGAACGCAT TAACTCATCC
TGAATTGTAG C
SEQ ID NO: 44:
5′-CAGGCAGCTG AGTATCGAGA AATTGACAAA
CGTATGAACA
SEQ ID NO: 45:
5′-TTATCAAACA CGGTAGGGAA AGTTCTCACT
CTGCATTATA
SEQ ID NO: 46:
5′-TGGTTCCTTA ACCACATTTC TTTTTTTTTT TTTCCAGGCT
ATTCAAGA
SEQ ID NO: 47:
5′-AGGAACTGAA AAAAGTAATT TAAATGGGAA
TAGCCCTTCA ATA
SEQ ID NO: 48:
5′-CTGGAAAAAA CTGTTTGGGA CCTCCGGTCA GAAAACCA
SEQ ID NO: 49:
5′-AGAGTAAAGT AGATGATGGA AATATACAGC
TTGCAAGGACT
SEQ ID NO: 50:
5′-GAGGATCTTG AAGGAAACTG AATTCAAAAA
GATCAAAGTG C
SEQ ID NO: 51:
5′-CAGGGTCTAG AGCAGAGCAG CTGCCAGACA
TGAGAAAAG
SEQ ID NO: 52:
5′-ATCCCAGAAG GTGAGAAAGT TAAAATTCCC
GTCGCTATCA
SEQ ID NO: 53:
5′-CTGCACACAC CAGTTGAGCA GGTACTGGGA GCCAATAT
SEQ ID NO: 54:
5′-GTGCCTCTCC CTCCCTCCAG GAAGCCTACG TGATGG
SEQ ID NO: 55:
5′-GCATCCTCCC CTGCATGTGT TAAACAATAC
AGCTAGTGGG
SEQ ID NO: 56:
5′-TACTGGTGAA AACACCGCAG CATGTCAAGA
TCACAGATTT
SEQ ID NO: 57:
5′-CTTTCTAGTA ACTCAGCAGC ATCTCAGGGC CAAAAATT
SEQ ID NO: 58:
5′-AAGACCTCAC AGTAAAAATA GGTGATTTTG GTCTAGCT
SEQ ID NO: 59:
5′-TAGTCCTACA GTGTTTTCAG TTTCAAAAAT
ACTTAACTCC TGTTAAATTA TAG
SEQ ID NO: 60:
5′-GCAAGCTTTC TCACAAGCAT TTGGTTTTAA
ATTATGGAGT ATG
Wild-type NRAS protein, p21 Ras:
MTEYKLVVVGAGGVGKSALTIQLIQNHFVDEYDPTIEDSYRKQVVIDGETCLLDILDTAG
QEEYSAMRDQYMRTGEGFLCVFAINNSKSFADINLYREQIKRVKDSDDVPMVLVGNKCDL
PTRTVDTKQAHELAKSYGIPFIETSAKTRQGVEDAFYTLVREIRQYRMKKLNSSDDGTQG
CMGLPCVVM
(SEQ ID NO: 61)
Wild-type Myeloid leukemia factor 2:
MFRFMRDVEPEDPMFLMDPFAIHRQHMSRMLSGGFGYSPFLSITDGNMPGTRPASRRMQQ
AGAVSPFGMLGMSGGFMDMFGMMNDMIGNMEHMTAGGNCQTFSSSTVISYSNTGDGAPKV
YQETSEMRSAPGGIRETRRTVRDSDSGLEQMSIGHHIRDRAHILQRSRNHRTGDQEERQD
YINLDESEAAAFDDEWRRETSRFRQQRPLEFRRLESSGAGGRRAEGPPRLAIQGPEDSPS
RQSRRYDW
(SEQ ID NO: 62)
Wild-type K-Ras:
MTEYKLVVVGAGGVGKSALTIQLIQNHFVDEYDPTIEDSYRKQVVIDGETCLLDILDTAG
QEEYSAMRDQYMRTGEGFLCVFAINNTKSFEDIHHYREQIKRVKDSEDVPMVLVGNKCDL
PSRTVDTKQAQDLARSYGIPFIETSAKTRQRVEDAFYTLVREIRQYRLKKISKEEKTPGC
VKIKKCIIM
(SEQ ID NO: 63)
Wild-type P53:
MDDLMLSPDDIEQWFTEDPGPDEAPRMPEAAPPVAPAPAAPTPAAPAPAPSWPLSSSVPS
QKTYQGSYGFRLGFLHSGTAKSVTCTYSPALNKMFCQLAKTCPVQLWVDSTPPPGTRVRA
MAIYKQSQHMTEVVRRCPHHERCSDSDGLAPPQHLIRVEGNLRVEYLDDRNTFRHSVVVP
YEPPEVGSDCTTIHYNYMCNSSCMGGMNRRPILTIITLEDSSGNLLGRNSFEVRVCACPG
RDRRTEEENLRKKGEPHHELPPGSTKRALPNNTSSSPQPKKKPLDGEYFTLQIRGRERFE
MFRELNEALELKDAQAGKEPGGSRAHSSHLKSKKGQSTSRHKKLMFKTEGPDSD
(SEQ ID NO: 64)
Wild-type receptor protein ErbB2:
MKLRLPASPETHLDMLRHLYQGCQVVQGNLELTYLPTNASLSFLQDIQEVQGYVLIAHNQ
VRQVPLQRLRIVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCKGSRCWGESSE
DCQSLTRTVCAGGCARCKGPLPTDCCHEQCAAGCTGPKHSDCLACLHFNHSGICELHCPA
LVTYNTDTFESMPNPEGRYTFGASCVTACPYNYLSTDVGSCTLVCPLHNQEVTAEDGTQR
CEKCSKPCARVCYGLGMEHLREVRAVTSANIQEFAGCKKIFGSLAFLPESFDGDPASNTA
PLQPEQLQVFETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRPEDECVGEGLA
CHQLCARGHCWGPGPTQCVNCSQFLRGQECVEECRVLQGLPREYVNARHCLPCHPECQPQ
NGSVTCFGPEADQCVACAHYKDPPFCVARCPSGVKPDLSYMPIWKFPDEEGACQPCPINC
THSCVDLDDKGCPAEQRASPLTSIISAVVGILLVVVLGVVFGILIKRRQQKIRKYTMRRL
LQETELVEPLTPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQLMPYGCLLDHV
RENRGRLGSQDLLNWCMQIAKGMSYLEDVRLVHRDLAARNVLVKSPNHVKITDFGLARLL
DIDETEYHADGGKVPIKWMALESILRRRFTHQSDVWSYGVTVWELMTFGAKPYDGIPARE
IPDLLEKGERLPQPPICTIDVYMIMVKCWMIDSECRPRFRELVSEFSRMARDPQRFVVIQ
NEDLGPASPLDSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQSLPTHDPSPLQ
RYSEDPTVPLPSETDGYVAPLTCSPQPEYVNQPDVRPQPPSPREGPLPAARPAGATLERP
KTLSPGKNGVVKDVFAFGGAVENPEYLTPQGGAAPQPHPPPAFSPAFDNLYYWDQDPPER
GAPPSTFKGTPTAENPEYLGLDVPV
(SEQ ID NO: 65)
Wild-type isocitrate dehydrogenase 1:
MSKKISGGSVVEMQGDEMTRIIWELIKEKLIFPYVELDLHSYDLGIENRDATNDQVTKDA
AEAIKKHNVGVKCATITPDEKRVEEFKLKQMWKSPNGTIRNILGGTVFREAIICKNIPRL
VSGWVKPIIIGRHAYGDQYRATDFVVPGPGKVEITYTPSDGTQKVTYLVHNFEEGGGVAM
GMYNQDKSIEDFAHSSFQMALSKGWPLYLSTKNTILKKYDGRFKDIFQEIYDKQYKSQFE
AQKIWYEHRLIDDMVAQAMKSEGGFIWACKNYDGDVQSDSVAQGYGSLGMMTSVLVCPDG
KTVEAEAAHGTVTRHYRMYQKGQETSTNPIASIFAWTRGLAHRAKLDNNKELAFFANALE
EVSIETIEAGFMTKDLAACIKGLPNVQRSDYLNTFEFMDKLGENLKIKLAQAKL
(SEQ ID NO: 66)
Wild-type phosphatidylinositol-4,5-bisphosphate
3-kinase catalytic subunit alpha:
MPPRPSSGELWGIHLMPPRILVECLLPNGMIVTLECLREATLITIKHELFKEARKYPLHQ
LLQDESSYIFVSVTQEAEREEFFDETRRLCDLRLFQPFLKVIEPVGNREEKILNREIGFA
IGMPVCEFDMVKDPEVQDFRRNILNVCKEAVDLRDLNSPHSRAMYVYPPNVESSPELPKH
IYNKLDKGQIIVVIWVIVSPNNDKQKYTLKINHDCVPEQVIAEAIRKKTRSMLLSSEQLK
LCVLEYQGKYILKVCGCDEYFLEKYPLSQYKYIRSCIMLGRMPNLMLMAKESLYSQLPMD
CFTMPSYSRRISTATPYMNGETSTKSLWVINSALRIKILCATYVNVNIRDIDKIYVRTGI
YHGGEPLCDNVNTQRVPCSNPRWNEWLNYDIYIPDLPRAARLCLSICSVKGRKGAKEEHC
PLAWGNINLFDYTDTLVSGKMALNLWPVPHGLEDLLNPIGVTGSNPNKETPCLELEFDWF
SSVVKFPDMSVIEEHANWSVSREAGFSYSHAGLSNRLARDNELRENDKEQLKAISTRDPL
SEITEQEKDFLWSHRHYCVTIPEILPKLLLSVKWNSRDEVAQMYCLVKDWPPIKPEQAME
LLDCNYPDPMVRGFAVRCLEKYLTDDKLSQYLIQLVQVLKYEQYLDNLLVRFLLKKALTN
QRIGHFFFWHLKSEMHNKTVSQRFGLLLESYCRACGMYLKHLNRQVEAMEKLINLTDILK
QEKKDETQKVQMKFLVEQMRRPDFMDALQGFLSPLNPAHQLGNLRLEECRIMSSAKRPLW
LNWENPDIMSELLFQNNEIIFKNGDDLRQDMLTLQIIRIMENIWQNQGLDLRMLPYGCLS
IGDCVGLIEVVRNSHTIMQIQCKGGLKGALQFNSHTLHQWLKDKNKGEIYDAAIDLFTRS
CAGYCVATFILGIGDRHNSNIMVKDDGQLFHIDFGHFLDHKKKKFGYKRERVPFVLTQDF
LIVISKGAQECTKTREFERFQEMCYKAYLAIRQHANLFINLFSMMLGSGMPELQSFDDIA
YIRKTLALDKTEQEALEYFMKQMNDAHHGGWTTKMDWIFHTIKQHALN
(SEQ ID NO: 67)
Wild-type KIT receptor tyrosine kinase:
MRGARGAWDFLCVLLLLLRVQTGSSQPSVSPGEPSPPSIHPGKSDLIVRVGDEIRLLCTD
PGFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAYH
RLCLHCSVDQEGKSVLSEKFILKVRPAFKAVPVVSVSKASYLLREGEEFTVTCTIKDVSS
SVYSTWKRENSQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIYMNRTFTDKWE
DYPKSENESNIRYVSELHLTRLKGTEGGTYTFLVSNSDVNAAIAFNVYVNTKPEILTYDR
LVNGMLQCVAAGFPEPTIDWYFCPGTEQRCSASVLPVDVQTLNSSGPPFGKLVVQSSIDS
SAFKHNGTVECKAYNDVGKTSAYFNFAFKGNNKEQIHPHTLFTPLLIGFVIVAGMMCIIV
MILTYKYLQKPMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNHMNIVNLLGAC
TIGGPTLVITEYCCYGDLLNFLRRKRDSFICSKQEDHAEAALYKNLLHSKESSCSDSTNE
YMDMKPGVSYVVPTKADKRRSVRIGSYIERDVTPAIMEDDELALDLEDLLSFSYQVAKGM
AFLASKNCIHRDLAARNILLTHGRITKICDFGLARDIKNDSNYVVKGNARLPVKWMAPES
IFNCVYTFESDVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPVVDHSVRINSV
GSTASSSQPLLVHDDV
(SEQ ID NO: 68)
Wild-type phosphoinositide-3-kinase regulatory subunit 1:
MHGDYTLTLRKGGNNKLIKIFHRDGKYGFSDPLTFSSVVELINHYRNESLAQYNPKLDVK
LLYPVSKYQQDQVVKEDNIEAVGKKLHEYNTQFQEKSREYDRLYEEYTRTSQEIQMKRTA
IEAFNETIKIFEEQCQTQERYSKEYIEKFKREGNEKEIQRIMHNYDKLKSRISEIIDSRR
RLEEDLKKQAAEYREIDKRMNSIKPDLIQLRKTRDQYLMWLTQKGVRQKKLNEWLGNENT
EDQYSLVEDDEDLPHHDEKTWNVGSSNRNKAENLLRGKRDGTFLVRESSKQGCYACSVVV
DGEVKHCVINKTATGYGFAEPYNLYSSLKELVLHYQHTSLVQHNDSLNVTLAYPVYAQQR
R
(SEQ ID NO: 69)
Wild-type nucleophosmin 1:
MEDSMDMDMSPLRPQNYLFGCELKADKDYHFKVDNDENEHQLSLRTVSLGAGAKDELHIV
EAEAMNYEGSPIKVTLATLKMSVQPTVSLGGFEITPPVVLRLKCGSGPVHISGQHLVAVE
EDAESEDEEEEDVKLLSISGKRSAPGGGSKVPQKKVKLAADEDDDDDDEEDDDEDDDDDD
FDDEEAEEKAPVKKSIRDTPAKNAQKSNQNGKDSKPSSTPRSKGQESFKKQEKTPKTPKG
PSSVEDIKAKMQASIEKAH
(SEQ ID NO: 70)
Wild-type epidermal growth factor receptor:
MRPSGTAGAALLALLAALCPASRALEEKKVCQGTSNKLTQLGTFEDHFLSLQRMENNCEV
VLGNLEITYVQRNYDLSFLKTIQEVAGYVLIALNTVERIPLENLQIIRGNMYYENSYALA
VLSNYDANKTGLKELPMRNLQGQKCDPSCPNGSCWGAGEENCQKLTKIICAQQCSGRCRG
KSPSDCCHNQCAAGCTGPRESDCLVCRKFRDEATCKDTCPPLMLYNPTTYQMDVNPEGKY
SFGATCVKKCPRNYVVTDHGSCVRACGADSYEMEEDGVRKCKKCEGPCRKVCNGIGIGEF
KDSLSINATNIKHFKNCTSISGDLHILPVAFRGDSFTHTPPLDPQELDILKTVKEITGFL
LIQAWPENRTDLHAFENLEIIRGRTKQHGQFSLAVVSLNITSLGLRSLKEISDGDVIISG
NKNLCYANTINWKKLFGTSGQKTKIISNRGENSCKATGQVCHALCSPEGCWGPEPRDCVS
CRNVSRGRECVDKCNLLEGEPREFVENSECIQCHPECLPQAMNITCTGRGPDNCIQCAHY
IDGPHCVKTCPAGVMGENNTLVWKYADAGHVCHLCHPNCTYGCTGPGLEGCPTNGPKIPS
IATGMVGALLLLLVVALGIGLFMRRRHIVRKRTLRRLLQERELVEPLTPSGEAPNQALLR
ILKETEFKKIKVLGSGAFGTVYKGLWIPEGEKVKIPVAIKELREATSPKANKEILDEAYV
MASVDNPHVCRLLGICLTSTVQLITQLMPFGCLLDYVREHKDNIGSQYLLNWCVQIAKGM
NYLEDRRLVHRDLAARNVLVKTPQHVKITDFGLAKLLGAEEKEYHAEGGKVPIKWMALES
ILHRIYTHQSDVWSYGVTVWELMTFGSKPYDGIPASEISSILEKGERLPQPPICTIDVYM
IMVKCWMIDADSRPKFRELIIEFSKMARDPQRYLVIQGDERMHLPSPTDSNFYRALMDEE
DMDDVVDADEYLIPQQGFFSSPSTSRTPLLSSLSATSNNSTVACIDRNGLQSCPIKEDSF
LQRYSSDPTGALTEDSIDDTFLPVPGEWLVWKQSCSSTSSTHSAAASLQCPSQVLPPASP
EGETVADLQTQ
(SEQ ID NO: 71)
Wild-type B-Raf:
MAALSGGGGGGAEPGQALFNGDMEPEAGAGAGAAASSAADPAIPEEVWNIKQMIKLTQEH
IEALLDKFGGEHNPPSIYLEAYEEYTSKLDALQQREQQLLESLGNGTDFSVSSSASMDTV
TSSSSSSLSVLPSSLSVFQNPTDVARSNPKSPQKPIVRVFLPNKQRTVVPARCGVTVRDS
LKKALMMRGLIPECCAVYRIQDGEKKPIGWDTDISWLTGEELHVEVLENVPLTTHNFVRK
TFFTLAFCDFCRKLLFQGFRCQTCGYKFHQRCSTEVPLMCVNYDQLDLLFVSKFFEHHPI
PQEEASLAETALTSGSSPSAPASDSIGPQILTSPSPSKSIPIPQPFRPADEDHRNQFGQR
DRSSSAPNVHINTIEPVNIDDLIRDQGFRGDGGSTTGLSATPPASLPGSLTNVKALQKSP
GPQRERKSSSSSEDRNRMKTLGRRDSSDDWEIPDGQITVGQRIGSGSFGTVYKGKWHGDV
AVKMLNVTAPTPQQLQAFKNEVGVLRKTRHVNILLFMGYSTKPQLAIVTQWCEGSSLYHH
LHIIETKFEMIKLIDIARQTAQGMDYLHAKSIIHRDLKSNNIFLHEDLTVKIGDFGLATV
KSRWSGSHQFEQLSGSILWMAPEVIRMQDKNPYSFQSDVYAFGIVLYELMTGQLPYSNIN
NRDQIIFMVGRGYLSPDLSKVRSNCPKAMKRLMAECLKKKRDERPLFPQILASIELLARS
LPKIHRSASEPSLNRAGFQTEDFSLYACASPKTPIQAGGYGAFPVH
(SEQ ID NO: 72)
Wild-type Janus kinase 2:
MGMACLTMTEMEGTSTSSIYQNGDISGNANSMKQIDPVLQVYLYHSLGKSEADYLTFPSG
EYVAEEICIAASKACGITPVYHNMFALMSETERIWYPPNHVFHIDESTRHNVLYRIRFYF
PRWYCSGSNRAYRHGISRGAEAPLLDDFVMSYLFAQWRHDFVHGWIKVPVTHETQEECLG
MAVLDMMRIAKENDQTPLAIYNSISYKTFLPKCIRAKIQDYHILTRKRIRYRFRRFIQQF
SQCKATARNLKLKYLINLETLQSAFYTEKFEVKEPGSGPSGEEIFATIIITGNGGIQWSR
GKHKESETLTEQDLQLYCDFPNIIDVSIKQANQEGSNESRVVTIHKQDGKNLEIELSSLR
EALSFVSLIDGYYRLTADAHHYLCKEVAPPAVLENIQSNCHGPISMDFAISKLKKAGNQT
GLYVLRCSPKDFNKYFLTFAVERENVIEYKHCLITKNENEEYNLSGTKKNFSSLKDLLNC
YQMETVRSDNIIFQFTKCCPPKPKDKSNLLVFRTNGVSDVPTSPTLQRPTHMNQMVFHKI
RNEDLIFNESLGQGTFTKIFKGVRREVGDYGQLHETEVLLKVLDKAHRNYSESFFEAASM
MSKLSHKHLVLNYGVCVCGDENILVQEFVKFGSLDTYLKKNKNCINILWKLEVAKQLAWA
MHFLEENTLIHGNVCAKNILLIREEDRKTGNPPFIKLSDPGISITVLPKDILQERIPWVP
PECIENPKNLNLATDKWSFGTTLWEICSGGDKPLSALDSQRKLQFYEDRHQLPAPKWAEL
ANLINNCMDYEPDFRPSFRAIIRDLNSLFTPDYELLTENDMLPNMRIGALGFSGAFEDRD
PTQFEERHLKFLQQLGKGNFGSVEMCRYDPLQDNTGEVVAVKKLQHSTEEHLRDFEREIE
ILKSLQHDNIVKYKGVCYSAGRRNLKLIMEYLPYGSLRDYLQKHKERIDHIKLLQYTSQI
CKGMEYLGTKRYIHRDLATRNILVENENRVKIGDFGLTKVLPQDKEYYKVKEPGESPIFW
YAPESLTESKFSVASDVWSFGVVLYELFTYIEKSKSPPAEFMRMIGNDKQGQMIVFHLIE
LLKNNGRLPRPDGCPDEIYMIMTECWNNNVNQRPSFRDLALRVDQIRDNMAG
(SEQ ID NO: 73)

What is claimed is:

1. A method of immobilizing a polynucleotide, the method comprising:
adding to a flow cell a first primer, a second primer, and a target polynucleotide, wherein the flow cell comprises a solid support and a third primer attached to the solid support, wherein the first primer comprises a first target binding sequence and a first platform primer binding sequence, the second primer comprises a second target binding sequence and a second platform primer sequence, and the third primer comprises a sequence complementary to the second platform primer sequence; and
within the flow cell:
a binding the first target binding sequence to the target polynucleotide and extending the first primer to generate a first complement of the target polynucleotide template comprising the first platform primer binding sequence and a complementary sequence of the target polynucleotide;
b) binding the second target binding sequence to the first complement of the target polynucleotide and extending the second primer to generate a second template comprising a first platform primer sequence, a sequence of the target polynucleotide, and the second platform primer sequence; and
c) binding the second platform primer sequence to the third primer and extending the third primer to form an immobilized polynucleotide

2. The method of claim 1, wherein the solid support comprises a plurality of primers attached to the solid support, wherein each of the primers comprise a sequence complementary to the second platform primer sequence.

3. The method of claim 1, wherein the solid support further comprises a fourth primer attached to the solid support, wherein said fourth primer comprises a sequence complementary to the first platform primer sequence.

4. The method of claim 1, wherein
a) occurs for about 1 minute, about 2 minutes, about 3 minutes, about 4 minutes, about 5 minutes, about 6 minutes, about 7 minutes, about 8 minutes, about 9 minutes, or about 10 minutes; and/or
b) occurs for about 1 minute, about 2 minutes, about 3 minutes, about 4 minutes, about 5 minutes, about 6 minutes, about 7 minutes, about 8 minutes, about 9 minutes, or about 10 minutes.

5. The method of claim 3, further comprising d) annealing the immobilized polynucleotide to the fourth primer; and e) extending the fourth primer to generate a complement of the immobilized polynucleotide.

6. The method of claim 1, further comprising sequencing the immobilized polynucleotide.

7. The method of claim 1, wherein the flow cell comprises a plurality of nanowells.

8. The method of claim 7, wherein each nanowell is about 0.1 μm to about 2 μm in depth, and wherein the nanowells are about 0.1 μm to about 2.0 μm in diameter.

9. The method of claim 1, wherein the target polynucleotide comprises a gene or gene fragment thereof.

10. The method of claim 9, wherein the target polynucleotide comprises an NRAS gene, or a portion thereof, a MLF2 gene, or a portion thereof, a KRAS gene, or a portion thereof, a TP53 gene, or a portion thereof, a ERBB2 gene, or a portion thereof, a IDH1 gene, or a portion thereof, a PIK3CA gene, or a portion thereof, a KIT gene, or a portion thereof, a PIK3R1 gene, or a portion thereof, a NPM1 gene, or a portion thereof, a EGFR gene, or a portion thereof, a BRAF gene, or a portion thereof, or a JAK2 gene, or a portion thereof.

11. The method of claim 9, wherein the target polynucleotide comprises a mutated NRAS gene, or a portion thereof, a mutated MLF2 gene, or a portion thereof, a mutated KRAS gene, or a portion thereof, a mutated TP53 gene, or a portion thereof, a mutated ERBB2 gene, or a portion thereof, a mutated IDH1 gene, or a portion thereof, a mutated PIK3CA gene, or a portion thereof, a mutated KIT gene, or a portion thereof, a mutated PIK3R1 gene, or a portion thereof, a mutated NPM1 gene, or a portion thereof, a mutated EGFR gene, or a portion thereof, a mutated BRAF gene, or a portion thereof, or a mutated JAK2 gene, or a portion thereof.

12. The method of claim 1, wherein the target polynucleotide is about 60 nucleotides to about 150 nucleotides in length.

13. The method of claim 1, wherein the target polynucleotide is about 80 nucleotides to about 120 nucleotides in length.

14. The method of claim 1, wherein the first target binding sequence is about 30, about 35, about 40, about 45, about 50, or about 55 nucleotides and wherein the second target binding sequence is about 30, about 35, about 40, about 45, about 50, or about 55 nucleotides.

15. The method of claim 1, wherein the third primer is about 10 nucleotides to about 35 nucleotides.

16. The method of claim 1, wherein the target polynucleotide does not comprise an adapter.

17. The method of claim 1, further comprising ligating the first platform primer binding sequence and the second platform primer sequence together to form a circular polynucleotide.

18. The method of claim 17, further comprising extending the third primer to generate an amplification product.

19. A method of amplifying a polynucleotide, the method comprising:
contacting a solid support of with a plurality of polynucleotides, wherein each of the plurality of polynucleotides do not include a common primer binding sequence and comprises a first platform primer binding sequence, a target sequence, and a second platform primer sequence, wherein the solid support comprises a first plurality of immobilized oligonucleotides each comprising a first platform primer sequence and a second plurality of immobilized oligonucleotides each comprising a second platform primer sequence;
hybridizing the first platform primer binding sequence of a polynucleotide of the plurality of polynucleotides to a first immobilized oligonucleotide from the first plurality, and extending with a polymerase the first immobilized oligonucleotide to generate a first extension product;
hybridizing the first extension product to a second immobilized oligonucleotide from the second plurality, and extending with a polymerase the second immobilized oligonucleotide to generate a second extension product; and
hybridizing the second extension product to a third immobilized oligonucleotide from the first plurality, and extending with a polymerase the third immobilized oligonucleotide to generate a third extension product.

20. The method of claim 19, further comprising sequencing the first extension product, the second extension product, and/or the third extension product.
